SEQUENCE LISTING (327 sequences)

SEQ ID NO: 1 - DNA, 4761 bp, Artificial Sequence
  Description of Artificial Sequence Synthetic polynucleotide
ggcctcacttgcagcagaacgtgggcagcttgctgaatcgttctgccaaagtgagcccgtaacataatggcgagtaatacgcattaaggcggtaactcagccccgcagggactagacctaacgttaggctcagcgctcgccgctctgatgctactgcatatccaaagctgctttagcactcgcagaagttcgcttgattgctcaagcgttcccgtcagtgaaatgatcctctttctgatagcgccagaaaaaactcccttcgtcctgccaagcccatttggaagtctcagcacacgcagagggtaacagcatttgtcatggatacgttcagcgcccaaggcgcggcgagagtcgagcaagcctcttatactgcgacagcggcaggtgagaacataagcgacgtagcgtgcggagtcgcgttgttagagcctgtccgctgtggtagacccccgtctagtattacgggggtaaatcccacagagcctgtgacactcaccttgtattcgcaagcgtagcgcgccagtgtttgagcgctagcgagtcttgctaagcaccatgatttaagatgctcttggtagaatgtcttatcagcatactttctaaaaccatgcttattgctttttgctcttcttcatctaactgttggattttttttaacctgagcataagctcttgattttcatctgtggcccatcttccgcatagttcatcaattgagatctccagagcatctgcgatcttcacaaggttttccattgtaggcaaaccttccccagattcgtattttttgtacgatgttagactaattccaatttcatcagccatttgtgcctgagtcttattaattgcctttctttggttggctagcctttcttttatcttcataacaatcccctttagcttgatttatttctattgtaaggctgtttttttgtacattagtcttgaaagtgcgcattggttgctgtattttagctctaaaggttatcttgacaggtttttgaaggctaatgaaaaagcagattttcactcttgacgaattacaactcgatacaaacgcttctccgtttgtttttgtcgattatcttgcttggtcggttccttatgcttcattccgtcacgcgcataagtccgatttgtcctcgcttatctgggcgcctcttcctaagcctgattaccgtatggctcgcacacctgagcaaaaagagaagttaatcgagctttataagcagaagtggaacgttgccatgatggaacgcttggaggtcttttgccttcatgttcttggtcttcgtatgtcgccttggcgcgataaggggctttatgggtatgaaaactcatgccatttgatgtctaagtactccaataaacacgtgggctttgttgcgctagggggaaaccgtaatacctgttacttccaaattgagggagtagggtgtcgaaccgtgttagagcacacctctttattccgtcttcattggtggctcgatttattaggttgctctcgtctgtctcgtattgatttagccgttgatgactttcacggtttgtttggccgtgagtacgccaaaaaagcctattccgatgacgcctttcgcaccgctagagcgggacgtgcccctaacggtggtgagcgattagtctctgagcctaatggcaaaatcatcaatgaatctttcgaggtaggctctcgtgaatctcgcatttactggcgtatctacaacaaggctgctcagcttggtttagatatgcactggtttcgtaatgaggtcgagcttaaagatatgcctatcgacgttctgctcaatatcgaggggtattttgcaggtttgtgcgcgtactcggcctcaattatcaattccttgcctgtcaaggtggtcacaaaaaagcgtcaagtggcgcttgatatccactcacgcattaagtgggctcgtcgtcaggtcggtaagacgttgtttgatatttcaaagcattttggtggtgatttggaaagggtgtttggggcgttgatttctaaggaaattcacgacgattcactcaaccttccagattcttatatgaagttaattgatgaaattatgggtgattaacagctgggcgcgccccatgtcagccgttaagtgttcctgtgtcactcaaaattgctttgagaggctctaagggcttctcagtgcgttacatccctggcttgttgtccacaaccgttaaaccttaaaggctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcgacgtcttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaataattaattggggaccctagaggtccccttttttattttaaaaattttttcacaaaacggtttacaagcataactagtgcggccgcaagcttgccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgacctttaggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataacggccttaattaaatgatgtttttattccacatccttagtgcgtattatgtggcgcgtcattatgttgaggggcagtcgtcagtaccattgcgccagcactgac

SEQ ID NO: 2 - DNA, 90 bp, Artificial Sequence
  Description of Artificial Sequence Synthetic oligonucleotide
cttgatgaaacaacgcggcgagctttgatcaacgaccttttggaaacttcggcttcccctggagagagcgagattctccgcgctgtagaa

SEQ ID NO: 3 - DNA, 90 bp, Artificial Sequence
  Description of Artificial Sequence Synthetic oligonucleotide
ttctacagcgcggagaatctcgctctctccaggggaagccgaagtttccaaaaggtcgttgatcaaagctcgccgcgttgtttcatcaag

SEQ ID NO: 4 - DNA, 90 bp, Artificial Sequence; identical to SEQ ID NO: 3
  Description of Artificial Sequence Synthetic oligonucleotide

SEQ ID NO: 5 - DNA, 90 bp, Artificial Sequence; identical to SEQ ID NO: 3
  Description of Artificial Sequence Synthetic oligonucleotide

SEQ ID NO: 6 - DNA, 90 bp, Artificial Sequence; identical to SEQ ID NO: 3
  Description of Artificial Sequence Synthetic oligonucleotide

SEQ ID NO: 7 - DNA, 90 bp, Artificial Sequence; identical to SEQ ID NO: 3
  Description of Artificial Sequence Synthetic oligonucleotide

SEQ ID NO: 8 - DNA, 90 bp, Artificial Sequence
  Description of Artificial Sequence Synthetic oligonucleotide
cagcatctcgtgagattctggaaccatatggtaaagatcgtccgtagctgattggtgtaacggtactaaccagcatggaacagagtgatt

SEQ ID NO: 9 - DNA, 90 bp, Artificial Sequence
  Description of Artificial Sequence Synthetic oligonucleotide
aatcactctgttccatgctggttagtaccgttacaccaatcagctacggacgatctttaccatatggttccagaatctcacgagatgctg

SEQ ID NO: 10 - DNA, 2145 bp, Artificial Sequence
  Description of Artificial Sequence Synthetic polynucleotide
aaagcgtaccttcagctcaatgagattcgccttaacccggttttatttaaagaaaacacccaagcgttcttgcaagaagtgataccgcatgaggtcgctcacttaatcacatatcaggtttacggtcgcgtccgtcctcatggcaaagagtggcaaaccgtaatggaatccgtatttaacgttccggccaaaaccacacatagtttcgaagtctcttccgttcaaggcaaaaccttcgaataccgatgtcgctgcacgacatatcccctttctattcgccgtcacaacaaagtgctgcgcaaacaagccgtgtattcgtgtcaaaaatgtcgtcagcctcttagcttcactggtgtccagctttcctaatcctcagttcaattaagtctcaataggaaatattgaccaacatttcttttgttattattaacttgcttattacgaaagctaatatctgagtgatagaatggataaagtcatactttttaaagactttaactccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatcctcaccaatcgcgacaatcgctaatctttctgtttgaggcgtttcatttactccaattgaaacgcctcttgccccttgttttttcgatggaaagcatccatgttaggaactaagtttattctcttgctggaaatctcatgcgtatccctcgaatttatcatccagaaaccattcaccaacttggtacactcgctttaagtgacgacgccgctggccatattggccgcgtacttcgtatgaaggaaggtcaggaagttctcctatttgacggtagtggtgcagagtttcccgcagttatcgcagaagtcagcaaaaagaatgtcctcgtagacatctctgagcgcgtagagaacagcattgaatcccctttggatcttcacctaggacaggtgatttcacgaggcgacaagatggagttcaccattcagaagtcagtcgaactcggagtaaataccatcactccccttatttctgaacgttgtggcgtaaagctcgatc

SEQ ID NO: 11 - protein, 272 aa, Vibrio cholerae
MEKPKLIQRFAERFSVDPNKLFDTLKATAFKQRDGSAPTNEQMMALLVVADQYGLNPFTKEIFAFPDKQAGIIPVVGVDGWSRIINQHDQFDGMEFKTSENKVSLDGAKECPEWMECIIYRRDRSHPVKITEYLDEVYRPPFEGNGKNGPYRVDGPWQTHTKRMLRHKSMIQCSRIAFGFVGIFDQDEAERIIEGQATHIVEPSVIPPEQVDDRTRGLVYKLIERAEASNAWNSALEYANEHFQGVELTFAKQEIFNAQQQAAKALTQPLAS

SEQ ID NO: 12 - protein, 338 aa, Vibrio cholerae
MKVIDLSQRTPAWHQWRIAGVTASEAPIIMGRSPYKTPWRLWAEKTGFVLPEDLSNNPNVLRGIRLEPQARRAFENAHNDFLLPLCAEADHNAIFRASFDGINDAGEPVELKCPCQSVFEDVQAHREQSEAYQLYWVQVQHQILVANSTRGWLVFYFEDQLIEFEIQRDAAFLTELQETALQFWELVQTKKEPSKCPEQDCFVPKGEAQYRWTSLSRQYCSAHAEVVRLENHIKSLKEEMRDAQSKLVAMMGNYAHADYAGVKLSRYMMAGTVDYKQLATDKLGELDEQVLAAYRKAPQERLRISTNKPEQPVETPIKISLEQENLVLPGDSPSSFYF

SEQ ID NO: 13 - DNA, 1318 bp, Artificial Sequence
  Description of Artificial Sequence Synthetic polynucleotide
atgaaaaaccaagtaacactcataggctatgttggctctgagccagagacgcgagcctatccatcaggtgatttagtgaccagcatttcactggccacttctgagaaatggcgcgaccgtcaatccaatgagctcaaagagcatacggaatggcatcgggtcgtttttcgagatcgtggtggattaaagttagggctcagggcaaaagatttaatccaaaaaggagcgaagctttttgttcaagggcctcagcgcacgcgctcatgggagaaagatggcattaagcatcgattgaccgaagtggacgcggacgagtttctgcttcttgataatgtgaacaaagcatctgagccatcagcggcggatgatgcaggctcccaaactaattgggcacaaacttatcctgaaccagatttttaaccgagcaaaaacgctttaacccagccgggagtactttcccgtcaggggcagactcccactttgattgtcggagtccacaatggaaaaaccaaagctaatccaacgctttgctgagcgctttagtgtcgatccaaacaaactgttcgataccctaaaagcaacagcatttaagcaacgtgacggtagtgcaccgaccaatgagcagatgatggcgctcttggtggttgcagatcagtacggcttgaaccctttcaccaaagagatttttgcgttccctgataagcaagctggaattattccagtggtaggtgtcgatggatggtctcgcatcatcaatcaacacgaccagtttgatggcatggagtttaagacttcagaaaacaaagtctccctggatggcgcgaaagaatgcccggaatggatggaatgcattatctaccggcgcgaccgttcgcacccagtcaaaatcactgaatacctggatgaagtctatcgaccgccttttgagggtaacggaaaaaatggcccttaccgtgtagatggtccatggcagacgcacactaagcgaatgctaagacataaatccatgatccagtgttcccgcattgcgtttggctttgtgggaattttcgatcaagacgaagcggagcgaattatcgaaggccaagcaacacacattgttgagccatcggtgattccacccgagcaagttgatgatcgaacccgagggcttgtttacaagcttatcgagcgggcggaagcttcaaacgcatggaatagtgcattggaatacgccaatgaacattttcaaggtgttgaactgacgtttgcgaaacaagaaatatttaatgcacagcaacaagcagccaaagcgctcacacagcctttagcttcttag

SEQ ID NO: 14 - protein, 139 aa, Vibrio cholerae
MKNQVTLIGYVGSEPETRAYPSGDLVTSISLATSEKWRDRQSNELKEHTEWHRVVFRDRGGLKLGLRAKDLIQKGAKLFVQGPQRTRSWEKDGIKHRLTEVDADEFLLLDNVNKASEPSAADDAGSQTNWAQTYPEPDF

SEQ ID NO: 15 - DNA, 420 bp, Vibrio cholerae
atgaaaaaccaagtaacactcataggctatgttggctctgagccagagacgcgagcctatccatcaggtgatttagtgaccagcatttcactggccacttctgagaaatggcgcgaccgtcaatccaatgagctcaaagagcatacggaatggcatcgggtcgtttttcgagatcgtggtggattaaagttagggctcagggcaaaagatttaatccaaaaaggagcgaagctttttgttcaagggcctcagcgcacgcgctcatgggagaaagatggcattaagcatcgattgaccgaagtggacgcggacgagtttctgcttcttgataatgtgaacaaagcatctgagccatcagcggcggatgatgcaggctcccaaactaattgggcacaaacttatcctgaaccagatttttaa

SEQ ID NO: 16 - protein, 138 aa, Artificial Sequence
  Description of Artificial Sequence Synthetic polypeptide
MDINTETEIKQKHSLTPFPVFLISPAFRGRYFHSYFRSSAMNAYYIQDRLEAQSWARHYQQLAREEKEAELADDMEKGLPQHLFESLCIDHLQRHGASKKSITRAFDDDVEFQERMAEHIRYMVETIAHHQVDIDSEV

SEQ ID NO: 17 - protein, 360 aa, Artificial Sequence
  Description of Artificial Sequence Synthetic polypeptide
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 18 - protein, 271 aa, Artificial Sequence
  Description of Artificial Sequence Synthetic polypeptide
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 19 - DNA, 10892 bp, Artificial Sequence
  Description of Artificial Sequence Synthetic polynucleotide
ggtaccagatctgcgggcagtgagcgcaacgcaattaatgtgagttagctcactcattaggcaccccaggctttacactttatgcttccggctcgtataatgtgtggaattgtgagcggataacaatttcacacaggaggatcccgatcgaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgaaaaaccaagtaacactcataggctatgttggctctgagccagagacgcgagcctatccatcaggtgatttagtgaccagcatttcactggccacttctgagaaatggcgcgaccgtcaatccaatgagctcaaagagcatacggaatggcatcgggtcgtttttcgagatcgtggtggattaaagttagggctcagggcaaaagatttaatccaaaaaggagcgaagctttttgttcaagggcctcagcgcacgcgctcatgggagaaagatggcattaagcatcgattgaccgaagtggacgcggacgagtttctgcttcttgataatgtgaacaaagcatctgagccatcagcggcggatgatgcaggctcccaaactaattgggcacaaacttatcctgaaccagatttttaaccgagcaaaaacgctttaacccagccgggagtactttcccgtcaggggcagactcccactttgattgtcggagtccacaatggaaaaaccaaagctaatccaacgctttgctgagcgctttagtgtcgatccaaacaaactgttcgataccctaaaagcaacagcatttaagcaacgtgatggtagtgcaccgaccaatgagcagatgatggcgctcttggtggttgcagatcagtacggcttgaaccctttcaccaaagagatttttgcgttccctgataagcaagctggaattattccagtggtaggtgtcgatggatggtctcgcatcatcaatcaacacgaccagtttgatggcatggagtttaagacttcagaaaacaaagtctccctggatggcgcgaaagaatgcccggaatggatggaatgcattatctaccggcgcgaccgttcgcacccagtcaaaatcactgaatacctggatgaagtctatcgaccgccttttgagggtaacggaaaaaatggcccttaccgtgtagatggtccatggcagacgcacactaagcgaatgctaagacataaatccatgatccagtgttcccgcattgcgtttggctttgtgggaattttcgatcaagacgaagcggagcgaattatcgaaggccaagcaacacacattgttgagccatcggtgattccacccgagcaagttgatgatcgaacccgagggcttgtttacaagcttatcgagcgggcggaagcttcaaacgcatggaatagtgcattggaatacgccaatgaacattttcaaggtgttgaactgacgtttgcgaaacaagaaatatttaatgcacagcaacaagcagccaaagcgctcacacagcctttagcttcttagcgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaagcggccgcgatcaagcaggtgcgacagacgtcatactagatatcaagcgacttctcctatcccctgggaacacatcaatctcaccggagaatatcgctggccaaagccttagcgtaggattccgccccttcccgcaaacgaccccaaacaggaaacgcagctgaaacgggaagctcaacacccactgacgcatgggttgttcaggcagtacttcatcaaccagcaaggcggcactttcggccatccgccgcgccccacagctcgggcagaaaccgcgacgcttacagctgaaagcgaccaggtgctcggcgtggcaagactcgcagcgaacccgtagaaagccatgctccagccgcccgcattggagaaattcttcaaattcccgttgcacatagcccggcaattcctttccctgctctgccataagcgcagcgaatgccgggtaatactcgtcaacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagcaacagcgaggcagcatggcgatttatcaccttacggcgaaaaccggcagcaggtcgggcggccaatcggccagggccaaggccgactacatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctccgatctgcgggcagtgagcgcaacgcaattaatgtgagttagctcactcattaggcaccccaggctttacactttatgcttccggctcgtataatgtgtggaattgtgagcggataacaatttcacacaggatctagaaataattttgtttaactttaagaaggagatatacatatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaaagctgatccgcggccgccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtagggataacagggtaattctagagtcgacctgcaggcatgcaagcttagatcctttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttg

SEQ ID NO: 20 - protein, 207 aa, Artificial Sequence
  Description of Artificial Sequence Synthetic polypeptide
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 21 - protein, 138 aa, Artificial Sequence; identical to SEQ ID NO: 16
  Description of Artificial Sequence Synthetic polypeptide

SEQ ID NO: 22 - protein, 219 aa, Artificial Sequence
  Description of Artificial Sequence Synthetic polypeptide
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 23 - DNA, 5308 bp, Artificial Sequence
  Description of Artificial Sequence Synthetic polynucleotide
acgtctcattttcgccagatatcgacgtcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaaagaattcaaaagatctaaagaggagaaaggatctatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgagcagactcccactttgattgtcggagtccacaatggaaaaaccaaagctaatccaacgctttgctgagcgctttagtgtcgatccaaacaaactgttcgataccctaaaagcaacagcatttaagcaacgtgatggtagtgcaccgaccaatgagcagatgatggcgctcttggtggttgcagatcagtacggcttgaaccctttcaccaaagagatttttgcgttccctgataagcaagctggaattattccagtggtaggtgtcgatggatggtctcgcatcatcaatcaacacgaccagtttgatggcatggagtttaagacttcagaaaacaaagtctccctggatggcgcgaaagaatgcccggaatggatggaatgcattatctaccggcgcgaccgttcgcacccagtcaaaatcactgaatacctggatgaagtctatcgaccgccttttgagggtaacggaaaaaatggcccttaccgtgtagatggtccatggcagacgcacactaagcgaatgctaagacataaatccatgatccagtgttcccgcattgcgtttggctttgtgggaattttcgatcaagacgaagcggagcgaattatcgaaggccaagcaacacacattgttgagccatcggtgattccacccgagcaagttgatgatcgaacccgagggcttgtttacaagcttatcgagcgggcggaagcttcaaacgcatggaatagtgcattggaatacgccaatgaacattttcaaggtgttgaactgacgtttgcgaaacaagaaatatttaatgcacagcaacaagcagccaaagcgctcacacagcctttagcttcttagctcgagtaaggaatgaaaaaccaagtaacactcataggctatgttggctctgagccagagacgcgagcctatccatcaggtgatttagtgaccagcatttcactggccacttctgagaaatggcgcgaccgtcaatccaatgagctcaaagagcatacggaatggcatcgggtcgtttttcgagatcgtggtggattaaagttagggctcagggcaaaagatttaatccaaaaaggagcgaagctttttgttcaagggcctcagcgcacgcgctcatgggagaaagatggcattaagcatcgattgaccgaagtggacgcggacgagtttctgcttcttgataatgtgaacaaagcatctgagccatcagcggcggatgatgcaggctcccaaactaattgggcacaaacttatcctgaaccagatttttaatctccaggcatatgaaggttatcgacctatcacaacgtactcctgcatggcaccagtggcgcattgcaggggttacggcatctgaagccccaattattatggggcgttcaccctacaaaacaccttggcgattatgggcagaaaaaactggattcgtattaccggaagacctgtcgaataatcctaatgtacttcgcggtataaggttggagcctcaagcaaggcgagcatttgagaatgcgcataatgactttcttctgccgttatgtgcagaagccgatcataacgcaatctttcgagccagctttgatggcatcaacgatgcgggcgagcccgttgaactgaaatgtccttgccagtcagtttttgaggatgtgcaagctcaccgagaacaaagcgaggcgtaccagttgtattgggtgcaagtacagcatcaaatactggtcgccaatagcacgcgaggttggttggttttctattttgaggatcaactgattgagtttgaaatacaacgagacgcggcgttcttaactgagttgcaagaaacagcgcttcagttttgggagttagtacagaccaaaaaagaaccgtcaaaatgccctgagcaagattgttttgttcccaagggtgaagcccaataccgttggacatcgctgtctcgacagtattgctcagcacatgccgaagtggtccgactggaaaatcacattaaatctttgaaagaggaaatgcgagacgctcagtcaaaattggtcgccatgatgggtaactacgctcatgccgactatgctggggtcaaactcagtcgctacatgatggcgggcacggtggactataagcaattggccaccgataaattaggcgagctggatgaacaggttttagccgcttaccgaaaagcgccacaagagcggttgcgtatcagcaccaataagccagagcagcccgttgaaacaccaatcaaaatcagccttgagcaagagaacttggttctgccaggtgactcgccgagctcattttatttttaacaaataaaacgaaaggctcagtcgaaagactgggcctttcgttttatctgttgtttgtcggtgaacgctctctactagagtcacactggctcaccttcgggtgggcctttctgcgtttatacctagggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatca

SEQ ID NO: 24 - DNA, 20 bp, Artificial Sequence
  Description of Artificial Sequence Synthetic oligonucleotide
gaattcattaaagaggagaa

SEQ ID NO: 25 - DNA, 20 bp, Artificial Sequence
  Description of Artificial Sequence Synthetic oligonucleotide
tttctcctctttaatgaatt

SEQ ID NO: 26 - DNA, 20 bp, Artificial Sequence
  Description of Artificial Sequence Synthetic oligonucleotide
acctgcggtaatggcgatgg

SEQ ID NO: 27 - DNA, 20 bp, Artificial Sequence
  Description of Artificial Sequence Synthetic oligonucleotide
tgaacctgcggtaatggcga

SEQ ID NO: 28 - DNA, 20 bp, Artificial Sequence
  Description of Artificial Sequence Synthetic oligonucleotide
taccattgaacctgcggtaa

SEQ ID NO: 29 - DNA, 20 bp, Artificial Sequence
  Description of Artificial Sequence Synthetic oligonucleotide
gattcgctaatatcgcatag

SEQ ID NO: 30 - DNA, 20 bp, Artificial Sequence
  Description of Artificial Sequence Synthetic oligonucleotide
aaataaatacggttgtggcc

SEQ ID NO: 31 - DNA, 20 bp, Artificial Sequence
  Description of Artificial Sequence Synthetic oligonucleotide
tatcaaaataaatacggttg

SEQ ID NO: 32 - DNA, 20 bp, Artificial Sequence
  Description of Artificial Sequence Synthetic oligonucleotide
ccaaacttttgcacgataag

SEQ ID NO: 33 - DNA, 20 bp, Artificial Sequence
  Description of Artificial Sequence Synthetic oligonucleotide
gccaaacttttgcacgataa

SEQ ID NO: 34 - DNA, 20 bp, Artificial Sequence
  Description of Artificial Sequence Synthetic oligonucleotide
cgccaaacttttgcacgata

SEQ ID NO: 35 - DNA, 20 bp, Artificial Sequence
  Description of Artificial Sequence Synthetic oligonucleotide
tgatagaattgctatttagc

SEQ ID NO: 36 - DNA, 20 bp, Artificial Sequence
  Description of Artificial Sequence Synthetic oligonucleotide
ttgatagaattgctatttag

SEQ ID NO: 37 - DNA, 20 bp, Artificial Sequence
  Description of Artificial Sequence Synthetic oligonucleotide
gtcgttgatcgcgcgcatct

SEQ ID NO: 38 - DNA, 20 bp, Artificial Sequence
  Description of Artificial Sequence Synthetic oligonucleotide
tggcgttgcgctatttgact

SEQ ID NO: 39 - DNA, 20 bp, Artificial Sequence
  Description of Artificial Sequence Synthetic oligonucleotide
ttccattgaagtattccagc

SEQ ID NO: 40 - DNA, 20 bp, Artificial Sequence
  Description of Artificial Sequence Synthetic oligonucleotide
gaataggtcttcaacttcac

SEQ ID NO: 41 - DNA, 20 bp, Artificial Sequence
  Description of Artificial Sequence Synthetic oligonucleotide
caatatcgaccgtcggttgt

SEQ ID NO: 42 - DNA, 20 bp, Artificial Sequence
  Description of Artificial Sequence Synthetic oligonucleotide
acaatatcgaccgtcggttg

SEQ ID NO: 43 - DNA, 20 bp, Artificial Sequence
  Description of Artificial Sequence Synthetic oligonucleotide
tgaccgacaatatcgaccgt

SEQ ID NO: 44 - DNA, 20 bp, Artificial Sequence
  Description of Artificial Sequence Synthetic oligonucleotide
gttttattgagcgttctgct

SEQ ID NO: 45 - DNA, 20 bp, Artificial Sequence
  Description of Artificial Sequence Synthetic oligonucleotide
tgtgcgctctcttgctatat

SEQ ID NO: 46 - DNA, 20 bp, Artificial Sequence
  Description of Artificial Sequence Synthetic oligonucleotide
ttgtgcgctctcttgctata

SEQ ID NO: 47 - DNA, 20 bp, Artificial Sequence
  Description of Artificial Sequence Synthetic oligonucleotide
ctgtgatggatagagtggcg

SEQ ID NO: 48 - DNA, 20 bp, Artificial Sequence
  Description of Artificial Sequence Synthetic oligonucleotide
gcaacctgtgatggatagag

SEQ ID NO: 49 - DNA, 20 bp, Artificial Sequence
  Description of Artificial Sequence Synthetic oligonucleotide
gcgttcaaagcaacctgtga

SEQ ID NO: 50 - DNA, 20 bp, Artificial Sequence
  Description of Artificial Sequence Synthetic oligonucleotide
tcatcgtcaacgacataaac

SEQ ID NO: 51 - DNA, 20 bp, Artificial Sequence
  Description of Artificial Sequence Synthetic oligonucleotide
aaaggcttgcccatcagcaa

SEQ ID NO: 52 - DNA, 20 bp, Artificial Sequence
  Description of Artificial Sequence Synthetic oligonucleotide
aatgtctaccgcatcgagaa

SEQ ID NO: 53 - DNA, 20 bp, Artificial Sequence
  Description of Artificial Sequence Synthetic oligonucleotide
gagaagcggtgtttttggta

SEQ ID NO: 54 - DNA, 20 bp, Artificial Sequence
  Description of Artificial Sequence Synthetic oligonucleotide
taattgagaagcggtgtttt

SEQ ID NO: 55 - DNA, 20 bp, Artificial Sequence
  Description of Artificial Sequence Synthetic oligonucleotide
ttgatttcataattgagaag

SEQ ID NO: 56 - DNA, 20 bp, Artificial Sequence
  Description of Artificial Sequence Synthetic oligonucleotide
tccactgctgagctgctgac

SEQ ID NO: 57 - DNA, 20 bp, Artificial Sequence
  Description of Artificial Sequence Synthetic oligonucleotide
aatagccatgtcgattgatt

SEQ ID NO: 58 - DNA, 20 bp, Artificial Sequence
  Description of Artificial Sequence Synthetic oligonucleotide
ctgccctaacacagcaaacg

SEQ ID NO: 59 - DNA, 20 bp, Artificial Sequence
  Description of Artificial Sequence Synthetic oligonucleotide
cgttcagttcttgtggaatg

SEQ ID NO: 60 - DNA, 20 bp, Artificial Sequence
  Description of Artificial Sequence Synthetic oligonucleotide
tacagctcgttcagttcttg

SEQ ID NO: 61 - DNA, 20 bp, Artificial Sequence
  Description of Artificial Sequence Synthetic oligonucleotide
cggcatattgctactgagtc

SEQ ID NO: 62 - DNA, 20 bp, Artificial Sequence
  Description of Artificial Sequence Synthetic oligonucleotide
agttctttaatattagtgac

SEQ ID NO: 63 - DNA, 20 bp, Artificial Sequence
  Description of Artificial Sequence Synthetic oligonucleotide
tttcttaacgcgagtgacga

SEQ ID NO: 64 - DNA, 20 bp, Artificial Sequence
  Description of Artificial Sequence Synthetic oligonucleotide
ctttcttaacgcgagtgacg

SEQ ID NO: 65 - DNA, 20 bp, Artificial Sequence
  Description of Artificial Sequence Synthetic oligonucleotide
cgattcgtcccatacaaatc

SEQ ID NO: 66 - DNA, 20 bp, Artificial Sequence
  Description of Artificial Sequence Synthetic oligonucleotide
agccgacttgttggccgtaa

SEQ ID NO: 67 - DNA, 20 bp, Artificial Sequence
  Description of Artificial Sequence Synthetic oligonucleotide
ctaagcgagagccgacttgt

SEQ ID NO: 68 - DNA, 20 bp, Artificial Sequence
  Description of Artificial Sequence Synthetic oligonucleotide
acttagtgccccaatagaaa

SEQ ID NO: 69 - DNA, 20 bp, Artificial Sequence
  Description of Artificial Sequence Synthetic oligonucleotide
tacttagtgccccaatagaa

SEQ ID NO: 70 - DNA, 20 bp, Artificial Sequence
  Description of Artificial Sequence Synthetic oligonucleotide
accacttgtcgaaaaagcaa

SEQ ID NO: 71 - DNA, 20 bp, Artificial Sequence
  Description of Artificial Sequence Synthetic oligonucleotide
attggctgaggtatttatgg

SEQ ID NO: 72 - DNA, 20 bp, Artificial Sequence
  Description of Artificial Sequence Synthetic oligonucleotide
acaattggctgaggtattta

SEQ ID NO: 73 - DNA, 20 bp, Artificial Sequence
  Description of Artificial Sequence Synthetic oligonucleotide
ctaagccggacaattggctg

SEQ ID NO: 74 - DNA, 20 bp, Artificial Sequence
  Description of Artificial Sequence Synthetic oligonucleotide
cttagcgatgcatctttagt

SEQ ID NO: 75 - DNA, 20 bp, Artificial Sequence
  Description of Artificial Sequence Synthetic oligonucleotide
gcttttatttccacttggtt

SEQ ID NO: 76 - DNA, 20 bp, Artificial Sequence
  Description of Artificial Sequence Synthetic oligonucleotide
aataggcttttatttccact

SEQ ID NO: 77 - DNA, 20 bp, Artificial Sequence
  Description of Artificial Sequence Synthetic oligonucleotide
gcgtctgggatgtatacatt

SEQ ID NO: 78 - DNA, 20 bp, Artificial Sequence
  Description of Artificial Sequence Synthetic oligonucleotide
ttttttccagaaacgcgtct

SEQ ID NO: 79 - DNA, 20 bp, Artificial Sequence
  Description of Artificial Sequence Synthetic oligonucleotide
attttttccagaaacgcgtc

SEQ ID NO: 80 - DNA, 20 bp, Artificial Sequence
  Description of Artificial Sequence Synthetic oligonucleotide
atcttcaccagcaaaagata

SEQ ID NO: 81 - DNA, 20 bp, Artificial Sequence
  Description of Artificial Sequence Synthetic oligonucleotide
tatcttcaccagcaaaagat

SEQ ID NO: 82 - DNA, 20 bp, Artificial Sequence
  Description of Artificial Sequence Synthetic oligonucleotide
accattattctttaacccag

SEQ ID NO: 83 - DNA, 20 bp, Artificial Sequence
  Description of Artificial Sequence Synthetic oligonucleotide
tcttgatcagccgctattgc

SEQ ID NO: 84 - DNA, 20 bp, Artificial Sequence
  Description of Artificial Sequence Synthetic oligonucleotide
aagtcttcaagaacttcgtt

SEQ ID NO: 85 - DNA, 20 bp, Artificial Sequence
  Description of Artificial Sequence Synthetic oligonucleotide
ttagatacgaaataggtaga

SEQ ID NO: 86 - DNA, 20 bp, Artificial Sequence
  Description of Artificial Sequence Synthetic oligonucleotide
aaagtagagcgcgattctac

SEQ ID NO: 87 - DNA, 20 bp, Artificial Sequence
  Description of Artificial Sequence Synthetic oligonucleotide
actgacggtgctgatataaa

SEQ ID NO: 88 - DNA, 20 bp, Artificial Sequence
  Description of Artificial Sequence Synthetic oligonucleotide
tctttgtcgagatagactga

SEQ ID NO: 89 - DNA, 20 bp, Artificial Sequence
  Description of Artificial Sequence Synthetic oligonucleotide
cctattgagtgcccacaatg

SEQ ID NO: 90 - DNA, 20 bp, Artificial Sequence
  Description of Artificial Sequence Synthetic oligonucleotide
aaactcttggtcaccattac

SEQ ID NO: 91 - DNA, 20 bp, Artificial Sequence
  Description of Artificial Sequence Synthetic oligonucleotide
ggcagtcatcataaaactct

SEQ ID NO: 92 - DNA, 20 bp, Artificial Sequence
  Description of Artificial Sequence Synthetic oligonucleotide
gggatgatgtatttaagata

SEQ ID NO: 93 - DNA, 20 bp, Artificial Sequence
  Description of Artificial Sequence Synthetic oligonucleotide
tgattaacggaattatcgtt

SEQ ID NO: 94 - DNA, 20 bp, Artificial Sequence
  Description of Artificial Sequence Synthetic oligonucleotide
atgattaacggaattatcgt

SEQ ID NO: 95 - DNA, 20 bp, Artificial Sequence
  Description of Artificial Sequence Synthetic oligonucleotide
catttgtaccacttttctcc

SEQ ID NO: 96 - DNA, 20 bp, Artificial Sequence
  Description of Artificial Sequence Synthetic oligonucleotide
tattttattagcctctctgt

SEQ ID NO: 97 - DNA, 20 bp, Artificial Sequence
  Description of Artificial Sequence Synthetic oligonucleotide
actagagcttttaccaagat

SEQ ID NO: 98 - DNA, 20 bp, Artificial Sequence
  Description of Artificial Sequence Synthetic oligonucleotide
ttaatctatctagctctgca

SEQ ID NO: 99 - DNA, 20 bp, Artificial Sequence
  Description of Artificial Sequence Synthetic oligonucleotide
taaccaccctcaagaatgca

SEQ ID NO: 100 - DNA, 20 bp, Artificial Sequence
  Description of Artificial Sequence Synthetic oligonucleotide
aaaaacaaacgcatccgtat

SEQ ID NO: 101 - DNA, 20 bp, Artificial Sequence
  Description of Artificial Sequence Synthetic oligonucleotide
caataccgctgcaggtttaa

SEQ ID NO: 102 - DNA, 20 bp, Artificial Sequence
  Description of Artificial Sequence Synthetic oligonucleotide
aaaagaccctgtttgtgcgg

SEQ ID NO: 103 - DNA, 20 bp, Artificial Sequence
  Description of Artificial Sequence Synthetic oligonucleotide
tgtaaaagaccctgtttgtg

SEQ ID NO: 104 - DNA, 20 bp, Artificial Sequence
  Description of Artificial Sequence Synthetic oligonucleotide
cgacatatttagaagtgatc

SEQ ID NO: 105 - DNA, 20 bp, Artificial Sequence
  Description of Artificial Sequence Synthetic oligonucleotide
ccgagtagtgccttaactac

SEQ ID NO: 106 - DNA, 20 bp, Artificial Sequence
  Description of Artificial Sequence Synthetic oligonucleotide
accgagtagtgccttaacta

SEQ ID NO: 107 - DNA, 20 bp, Artificial Sequence
  Description of Artificial Sequence Synthetic oligonucleotide
taatttgcagtgggtaacga

SEQ ID NO: 108 - DNA, 20 bp, Artificial Sequence
  Description of Artificial Sequence Synthetic oligonucleotide
gtaggtgccgaaattagcat

SEQ ID NO: 109 - DNA, 20 bp, Artificial Sequence
  Description of Artificial Sequence Synthetic oligonucleotide
tccttttgttgtgcgaacgt

SEQ ID NO: 110 - DNA, 20 bp, Artificial Sequence
  Description of Artificial Sequence Synthetic oligonucleotide
tgacatcctgcacaatcgca

SEQ ID NO: 111 - DNA, 20 bp, Artificial Sequence
  Description of Artificial Sequence Synthetic oligonucleotide
tacaacggagcgtgacgggt

SEQ ID NO: 112 - DNA, 20 bp, Artificial Sequence
  Description of Artificial Sequence Synthetic oligonucleotide
tcgatacaacggagcgtgac

SEQ ID NO: 113 - DNA, 20 bp, Artificial Sequence
  Description of Artificial Sequence Synthetic oligonucleotide
gtcgatacaacggagcgtga

SEQ ID NO: 114 - DNA, 20 bp, Artificial Sequence
  Description of Artificial Sequence Synthetic oligonucleotide
ttttgttatgaacttgtgat

SEQ ID NO: 115 - DNA, 20 bp, Artificial Sequence
  Description of Artificial Sequence Synthetic oligonucleotide
tttttgttatgaacttgtga

SEQ ID NO: 116 - DNA, 20 bp, Artificial Sequence
  Description of Artificial Sequence Synthetic oligonucleotide
tcatgaaaatcagagtttga

SEQ ID NO: 117 - DNA, 20 bp, Artificial Sequence
  Description of Artificial Sequence Synthetic oligonucleotide
atgccagcaaaccgaacttg

SEQ ID NO: 118 - DNA, 20 bp, Artificial Sequence
  Description of Artificial Sequence Synthetic oligonucleotide
attcaccgggtttgatgggg

SEQ ID NO: 119 - DNA, 20 bp, Artificial Sequence
  Description of Artificial Sequence Synthetic oligonucleotide
ctcattcaccgggtttgatg

SEQ ID NO: 120 - DNA, 20 bp, Artificial Sequence
  Description of Artificial Sequence Synthetic oligonucleotide
tggtattcgcatacgcatta

SEQ ID NO: 121 - DNA, 20 bp, Artificial Sequence
  Description of Artificial Sequence Synthetic oligonucleotide
ctggtattcgcatacgcatt

SEQ ID NO: 122 - DNA, 20 bp, Artificial Sequence
  Description of Artificial Sequence Synthetic oligonucleotide
atctagttccgtcaatgaac

SEQ ID NO: 123 - DNA, 20 bp, Artificial Sequence
  Description of Artificial Sequence Synthetic oligonucleotide
gagataaaccacgcatggtt

SEQ ID NO: 124 - DNA, 20 bp, Artificial Sequence
  Description of Artificial Sequence Synthetic oligonucleotide
tcgttgagataaaccacgca

SEQ ID NO: 125 - DNA, 20 bp, Artificial Sequence
  Description of Artificial Sequence Synthetic oligonucleotide
ctgttcaatttgagagatca

SEQ ID NO: 126 - DNA, 20 bp, Artificial Sequence
  Description of Artificial Sequence Synthetic oligonucleotide
aagtgtggtttctcgccaag

SEQ ID NO: 127 - DNA, 20 bp, Artificial Sequence
  Description of Artificial Sequence Synthetic oligonucleotide
caatcaatcaatgaaaagtg

SEQ ID NO: 128 - DNA, 20 bp, Artificial Sequence
  Description of Artificial Sequence Synthetic oligonucleotide
caataaaacttgggaaaaag

SEQ ID NO: 129 - DNA, 20 bp, Artificial Sequence
  Description of Artificial Sequence Synthetic oligonucleotide
actccaccccttgattttca

SEQ ID NO: 130 - DNA, 20 bp, Artificial Sequence
  Description of Artificial Sequence Synthetic oligonucleotide
attttttcgacttcgacagt

SEQ ID NO: 131 - DNA, 20 bp, Artificial Sequence
  Description of Artificial Sequence Synthetic oligonucleotide
aacatataagaagggaacag

SEQ ID NO: 132 - DNA, 20 bp, Artificial Sequence
  Description of Artificial Sequence Synthetic oligonucleotide
ctcttaatgctcggatggaa

SEQ ID NO: 133 - DNA, 20 bp, Artificial Sequence
  Description of Artificial Sequence Synthetic oligonucleotide
gctcttaatgctcggatgga

SEQ ID NO: 134 - DNA, 20 bp, Artificial Sequence
  Description of Artificial Sequence Synthetic oligonucleotide
aaaagctcttaatgctcgga

SEQ ID NO: 135 - DNA, 20 bp, Artificial Sequence
  Description of Artificial Sequence Synthetic oligonucleotide
tttttgagtagcgataattc

SEQ ID NO: 136 - DNA, 20 bp, Artificial Sequence
  Description of Artificial Sequence Synthetic oligonucleotide
ctcacaactggcaacaaaat

SEQ ID NO: 137 - DNA, 20 bp, Artificial Sequence
  Description of Artificial Sequence Synthetic oligonucleotide
ctgaataaatggctcacaac

SEQ ID NO: 138 - DNA, 20 bp, Artificial Sequence
  Description of Artificial Sequence Synthetic oligonucleotide
tcccatccaaataccattca

SEQ ID NO: 139 - DNA, 20 bp, Artificial Sequence
  Description of Artificial Sequence Synthetic oligonucleotide
gcgtatttaacccctgacag

SEQ ID NO: 140 - DNA, 20 bp, Artificial Sequence
  Description of Artificial Sequence Synthetic oligonucleotide
acatggtgtttgagagcgat

SEQ ID NO: 141 - DNA, 20 bp, Artificial Sequence
  Description of Artificial Sequence Synthetic oligonucleotide
caatttggcttattaccaca

SEQ ID NO: 142 - DNA, 20 bp, Artificial Sequence
  Description of Artificial Sequence Synthetic oligonucleotide
tcttgggtggatacgcaatt

SEQ ID NO: 143 - DNA, 20 bp, Artificial Sequence
  Description of Artificial Sequence Synthetic oligonucleotide
ctgaatcagagccaaaataa

SEQ ID NO: 144 - DNA, 20 bp, Artificial Sequence
  Description of Artificial Sequence Synthetic oligonucleotide
aagtgtgcggagctggaaat

SEQ ID NO: 145 - DNA, 20 bp, Artificial Sequence
  Description of Artificial Sequence Synthetic oligonucleotide
caggatcaagtgtgcggagc

SEQ ID NO: 146 - DNA, 20 bp, Artificial Sequence
  Description of Artificial Sequence Synthetic oligonucleotide
aatagaacccagtaaatagg

SEQ ID NO: 147 - DNA, 20 bp, Artificial Sequence
  Description of Artificial Sequence Synthetic oligonucleotide
ggaaatagaacccagtaaat

SEQ ID NO: 148 - DNA, 20 bp, Artificial Sequence
  Description of Artificial Sequence Synthetic oligonucleotide
acgacaaatcaacaccgcac

SEQ ID NO: 149 - DNA, 20 bp, Artificial Sequence
  Description of Artificial Sequence Synthetic oligonucleotide
aataccgtcaccaggtagaa

SEQ ID NO: 150 - DNA, 20 bp, Artificial Sequence
  Description of Artificial Sequence Synthetic oligonucleotide
tcagggccaataccgtcacc

SEQ ID NO: 151 - DNA, 20 bp, Artificial Sequence
  Description of Artificial Sequence Synthetic oligonucleotide
gcgcttgtgccatcacttca

SEQ ID NO: 152 - DNA, 20 bp, Artificial Sequence
  Description of Artificial Sequence Synthetic oligonucleotide
gcgacccctatgcctaattt

SEQ ID NO: 153 - DNA, 20 bp, Artificial Sequence
  Description of Artificial Sequence Synthetic oligonucleotide
cgcgacccctatgcctaatt

SEQ ID NO: 154 - DNA, 20 bp, Artificial Sequence
  Description of Artificial Sequence Synthetic oligonucleotide
actcagcgcagaaagaacaa

SEQ ID NO: 155 - DNA, 20 bp, Artificial Sequence
  Description of Artificial Sequence Synthetic oligonucleotide
cacgatgcgcttattggctc

SEQ ID NO: 156 - DNA, 20 bp, Artificial Sequence
  Description of Artificial Sequence Synthetic oligonucleotide
tcacgatgcgcttattggct

SEQ ID NO: 157 - DNA, 20 bp, Artificial Sequence
  Description of Artificial Sequence Synthetic oligonucleotide
cacactcacgatgcgcttat

SEQ ID NO: 158 - DNA, 20 bp, Artificial Sequence
  Description of Artificial Sequence Synthetic oligonucleotide
ttgttggatttgttcgtatt

SEQ ID NO: 159 - DNA, 20 bp, Artificial Sequence
  Description of Artificial Sequence Synthetic oligonucleotide
gtgattctagtaactcttgt

SEQ ID NO: 160 - DNA, 20 bp, Artificial Sequence
  Description of Artificial Sequence Synthetic oligonucleotide
ccagttactaaccatgtttt

SEQ ID NO: 161 - DNA, 20 bp, Artificial Sequence
  Description of Artificial Sequence Synthetic oligonucleotide
tcaaggttattaaattgctg

SEQ ID NO: 162 - DNA, 20 bp, Artificial Sequence
  Description of Artificial Sequence Synthetic oligonucleotide
cttggctatttagaaagtca

SEQ ID NO: 163 - DNA, 20 bp, Artificial Sequence
  Description of Artificial Sequence Synthetic oligonucleotide
cagcctcaaatttatctgct

SEQ ID NO: 164 - DNA, 20 bp, Artificial Sequence
  Description of Artificial Sequence Synthetic oligonucleotide
gcatacggcaccaaataaac

SEQ ID NO: 165 - DNA, 20 bp, Artificial Sequence
  Description of Artificial Sequence Synthetic oligonucleotide
cgtcaattcattagcgcata

SEQ ID NO: 166 - DNA, 20 bp, Artificial Sequence
  Description of Artificial Sequence Synthetic oligonucleotide
aaatagctttgtcgtttgta

SEQ ID NO: 167 - DNA, 20 bp, Artificial Sequence
  Description of Artificial Sequence Synthetic oligonucleotide
ttccatcggcgacgacgagc

SEQ ID NO: 168 - DNA, 20 bp, Artificial Sequence
  Description of Artificial Sequence Synthetic oligonucleotide
ggataaggatattgttccat

SEQ ID NO: 169 - DNA, 20 bp, Artificial Sequence
  Description of Artificial Sequence Synthetic oligonucleotide
aagcgataacacctaggata

SEQ ID NO: 170 - DNA, 20 bp, Artificial Sequence
  Description of Artificial Sequence Synthetic oligonucleotide
ggatacggtggtagcgttct

SEQ ID NO: 171 - DNA, 20 bp, Artificial Sequence
  Description of Artificial Sequence Synthetic oligonucleotide
tcaggacttggtggatacgg

SEQ ID NO: 172 - DNA, 20 bp, Artificial Sequence
  Description of Artificial Sequence Synthetic oligonucleotide
ctttcaggacttggtggata

SEQ ID NO: 173 - DNA, 20 bp, Artificial Sequence
  Description of Artificial Sequence Synthetic oligonucleotide
tgaacatcctacggttaata

SEQ ID NO: 174 - DNA, 20 bp, Artificial Sequence
  Description of Artificial Sequence Synthetic oligonucleotide
cgcagaaaatgaacatccta

SEQ ID NO: 175 - DNA, 20 bp, Artificial Sequence
  Description of Artificial Sequence Synthetic oligonucleotide
acacccttttagttcttctt

SEQ ID NO: 176 - DNA, 20 bp, Artificial Sequence
  Description of Artificial Sequence Synthetic oligonucleotide
caggtcgcttttcgttccag

SEQ ID NO: 177 - DNA, 20 bp, Artificial Sequence
  Description of Artificial Sequence Synthetic oligonucleotide
gtgactatcttggtgatctc

SEQ ID NO: 178 - DNA, 20 bp, Artificial Sequence
  Description of Artificial Sequence Synthetic oligonucleotide
ggcggcccaggtgactatct

SEQ ID NO: 179 - DNA, 20 bp, Artificial Sequence
  Description of Artificial Sequence Synthetic oligonucleotide
aaaccaagcggagctaagtg

SEQ ID NO: 180 - DNA, 20 bp, Artificial Sequence
  Description of Artificial Sequence Synthetic oligonucleotide
caaagcaacgataaaccaag

SEQ ID NO: 181 - DNA, 20 bp, Artificial Sequence
  Description of Artificial Sequence Synthetic oligonucleotide
cgcgacaacgaggattgcaa

SEQ ID NO: 182 - DNA, 20 bp, Artificial Sequence
  Description of Artificial Sequence Synthetic oligonucleotide
gagctgccgctgcatttgat

SEQ ID NO: 183 - DNA, 20 bp, Artificial Sequence
  Description of Artificial Sequence Synthetic oligonucleotide
ttaaagcgctcccatgcaga

SEQ ID NO: 184 - DNA, 20 bp, Artificial Sequence
  Description of Artificial Sequence Synthetic oligonucleotide
ggatcagttggtgccagaat

SEQ ID NO: 185 - DNA, 20 bp, Artificial Sequence
  Description of Artificial Sequence Synthetic oligonucleotide
ctggatgtctgggaataaaa

SEQ ID NO: 186 - DNA, 20 bp, Artificial Sequence
  Description of Artificial Sequence Synthetic oligonucleotide
catcccaggactggatgtct

SEQ ID NO: 187 - DNA, 20 bp, Artificial Sequence
  Description of Artificial Sequence Synthetic oligonucleotide
tcatcccaggactggatgtc

SEQ ID NO: 188 - DNA, 20 bp, Artificial Sequence
  Description of Artificial Sequence Synthetic oligonucleotide
gctggccagagtcagcagga

SEQ ID NO: 189 - DNA, 20 bp, Artificial Sequence
  Description of Artificial Sequence Synthetic oligonucleotide
gaatgctggccagagtcagc

SEQ ID NO: 190 - DNA, 20 bp, Artificial Sequence
  Description of Artificial Sequence Synthetic oligonucleotide
taaagcaataaagtgaatgc

SEQ ID NO: 191 - DNA, 20 bp, Artificial Sequence
  Description of Artificial Sequence Synthetic oligonucleotide
aggttcggctaacaaggtcg

SEQ ID NO: 192 - DNA, 20 bp, Artificial Sequence
  Description of Artificial Sequence Synthetic oligonucleotide
gtctgaaggttcggctaaca

SEQ ID NO: 193 - DNA, 20 bp, Artificial Sequence
  Description of Artificial Sequence Synthetic oligonucleotide
tacaggcgagtctgaaggtt

SEQ ID NO: 194 - DNA, 20 bp, Artificial Sequence
  Description of Artificial Sequence Synthetic oligonucleotide
tgaaaaccacagtcagggca

SEQ ID NO: 195 - DNA, 20 bp, Artificial Sequence
  Description of Artificial Sequence Synthetic oligonucleotide
tgaactgaaaaccacagtca

SEQ ID NO: 196 - DNA, 20 bp, Artificial Sequence
  Description of Artificial Sequence Synthetic oligonucleotide
ttgaactgaaaaccacagtc

SEQ ID NO: 197 - DNA, 20 bp, Artificial Sequence
  Description of Artificial Sequence Synthetic oligonucleotide
agagaaaaacccatcggtat

SEQ ID NO: 198 - DNA, 20 bp, Artificial Sequence
  Description of Artificial Sequence Synthetic oligonucleotide
atgaacagagaaaaacccat

SEQ ID NO: 199 - DNA, 20 bp, Artificial Sequence
  Description of Artificial Sequence Synthetic oligonucleotide
gcctaaaggccgttgccaca

SEQ ID NO: 200 - DNA, 20 bp, Artificial Sequence
  Description of Artificial Sequence Synthetic oligonucleotide
aaaatagccgagcggagcac

SEQ ID NO: 201 - DNA, 20 bp, Artificial Sequence
  Description of Artificial Sequence Synthetic oligonucleotide
aagttaataaaatagccgag

SEQ ID NO: 202 - DNA, 20 bp, Artificial Sequence
  Description of Artificial Sequence Synthetic oligonucleotide
ggaagaggtgagagggaacg

SEQ ID NO: 203 - DNA, 20 bp, Artificial Sequence
  Description of Artificial Sequence Synthetic oligonucleotide
atctggttggcgagcctggc

SEQ ID NO: 204 - DNA, 20 bp, Artificial Sequence
  Description of Artificial Sequence Synthetic oligonucleotide
tgctatctggttggcgagcc

SEQ ID NO: 205 - DNA, 20 bp, Artificial Sequence
  Description of Artificial Sequence Synthetic oligonucleotide
ctcccctgctgctatctggt

SEQ ID NO: 206 - DNA, 20 bp, Artificial Sequence
  Description of Artificial Sequence Synthetic oligonucleotide
tcatagacctctagaaacgt

SEQ ID NO: 207 - DNA, 20 bp, Artificial Sequence
  Description of Artificial Sequence Synthetic oligonucleotide
gtcggtggagctgatcgaag

SEQ ID NO: 208 - DNA, 20 bp, Artificial Sequence
  Description of Artificial Sequence Synthetic oligonucleotide
ttggtcccattttgggtcgg

SEQ ID NO: 209 - DNA, 20 bp, Artificial Sequence
  Description of Artificial Sequence Synthetic oligonucleotide
catttgagtgatgtgcattt

SEQ ID NO: 210 - DNA, 20 bp, Artificial Sequence
  Description of Artificial Sequence Synthetic oligonucleotide
actcatgtaagctcacaccg

SEQ ID NO: 211 - DNA, 20 bp, Artificial Sequence
  Description of Artificial Sequence Synthetic oligonucleotide
ggggtgaactaccacgtaat

SEQ ID NO: 212 - DNA, 20 bp, Artificial Sequence
  Description of Artificial Sequence Synthetic oligonucleotide
acctgaccatctcgcgtcca

SEQ ID NO: 213 - DNA, 20 bp, Artificial Sequence
  Description of Artificial Sequence Synthetic oligonucleotide
tacctgaccatctcgcgtcc

SEQ ID NO: 214 - DNA, 20 bp, Artificial Sequence
  Description of Artificial Sequence Synthetic oligonucleotide
aggcgcaactttgatgacat

SEQ ID NO: 215 - DNA, 20 bp, Artificial Sequence
  Description of Artificial Sequence Synthetic oligonucleotide
ttatgcagctccgggtccgg

SEQ ID NO: 216 - DNA, 20 bp, Artificial Sequence
  Description of Artificial Sequence Synthetic oligonucleotide
ttgttatgcagctccgggtc

SEQ ID NO: 217 - DNA, 20 bp, Artificial Sequence
  Description of Artificial Sequence Synthetic oligonucleotide
ctatcttgttatgcagctcc

SEQ ID NO: 218 - DNA, 20 bp, Artificial Sequence
  Description of Artificial Sequence Synthetic oligonucleotide
acgctgctgttgctccagac

SEQ ID NO: 219 - DNA, 20 bp, Artificial Sequence
  Description of Artificial Sequence Synthetic oligonucleotide
tgccaaaccgacagtattga

SEQ ID NO: 220 - DNA, 20 bp, Artificial Sequence
  Description of Artificial Sequence Synthetic oligonucleotide
ttatctttgtcttttaggta

SEQ ID NO: 221 - DNA, 20 bp, Artificial Sequence
  Description of Artificial Sequence Synthetic oligonucleotide
caatggctactttgccttca

SEQ ID NO: 222 - DNA, 20 bp, Artificial Sequence
  Description of Artificial Sequence Synthetic oligonucleotide
agtgtcacaacccgtaacaa

SEQ ID NO: 223 - DNA, 20 bp, Artificial Sequence
  Description of Artificial Sequence Synthetic oligonucleotide
ctaaaccaagcgccatacct

SEQ ID NO: 224 - DNA, 20 bp, Artificial Sequence
  Description of Artificial Sequence Synthetic oligonucleotide
acgacaagttctttaggttc

SEQ ID NO: 225 - DNA, 20 bp, Artificial Sequence
  Description of Artificial Sequence Synthetic oligonucleotide
tgcgtaacgacaagttcttt

SEQ ID NO: 226 - DNA, 20 bp, Artificial Sequence
  Description of Artificial Sequence Synthetic oligonucleotide
agtaaggcgctgcatgtttg

SEQ ID NO: 227 - DNA, 20 bp, Artificial Sequence
  Description of Artificial Sequence Synthetic oligonucleotide
ccccgagcaggattaatata

SEQ ID NO: 228 - DNA, 20 bp, Artificial Sequence
  Description of Artificial Sequence Synthetic oligonucleotide
taaacgcgatcaccccgagc

SEQ ID NO: 229 - DNA, 20 bp, Artificial Sequence
  Description of Artificial Sequence Synthetic oligonucleotide
atgtaacttaaatttggtgg

SEQ ID NO: 230 - DNA, 20 bp, Artificial Sequence
  Description of Artificial Sequence Synthetic oligonucleotide
ctctggtaagcagattgctt

SEQ ID NO: 231 - DNA, 20 bp, Artificial Sequence
  Description of Artificial Sequence Synthetic oligonucleotide
agatggggaattcctaactc

SEQ ID NO: 232 - DNA, 20 bp, Artificial Sequence
  Description of Artificial Sequence Synthetic oligonucleotide
tcaatacgagcttcaagatg

SEQ ID NO: 233 - DNA, 20 bp, Artificial Sequence
  Description of Artificial Sequence Synthetic oligonucleotide
agtcgttagcgcaacggcca

SEQ ID NO: 234 - DNA, 20 bp, Artificial Sequence
  Description of Artificial Sequence Synthetic oligonucleotide
caaagaagtcgttagcgcaa

SEQ ID NO: 235 - DNA, 20 bp, Artificial Sequence
  Description of Artificial Sequence Synthetic oligonucleotide
gattcatcaaatcccatcgg

SEQ ID NO: 236 - DNA, 20 bp, Artificial Sequence
  Description of Artificial Sequence Synthetic oligonucleotide
acaagcagcttgtcatttaa

SEQ ID NO: 237 - DNA, 20 bp, Artificial Sequence
  Description of Artificial Sequence Synthetic oligonucleotide
tgcaatgaccttacctcggt

SEQ ID NO: 238 - DNA, 20 bp, Artificial Sequence
  Description of Artificial Sequence Synthetic oligonucleotide
caactgcaatgaccttacct

SEQ ID NO: 239 - DNA, 20 bp, Artificial Sequence
  Description of Artificial Sequence Synthetic oligonucleotide
ctagaaataggggttctact

SEQ ID NO: 240 - DNA, 20 bp, Artificial Sequence
  Description of Artificial Sequence Synthetic oligonucleotide
tgcatgaattctagaaatag

SEQ ID NO: 241 - DNA, 20 bp, Artificial Sequence
  Description of Artificial Sequence Synthetic oligonucleotide
ctgcatgaattctagaaata

SEQ ID NO: 242 - DNA, 20 bp, Artificial Sequence
  Description of Artificial Sequence Synthetic oligonucleotide
cagcgcgcgaacttttggtc

SEQ ID NO: 243 - DNA, 20 bp, Artificial Sequence
  Description of Artificial Sequence Synthetic oligonucleotide
taatccagcgcgcgaacttt

SEQ ID NO: 244 - DNA, 20 bp, Artificial Sequence
  Description of Artificial Sequence Synthetic oligonucleotide
ttagaaagtaagcaaagaca

SEQ ID NO: 245 - DNA, 20 bp, Artificial Sequence
  Description of Artificial Sequence Synthetic oligonucleotide
gagtggcgtctttttgaatc

SEQ ID NO: 246 - DNA, 20 bp, Artificial Sequence
  Description of Artificial Sequence Synthetic oligonucleotide
gagatcagccgtcgtcagag

SEQ ID NO: 247 - DNA, 20 bp, Artificial Sequence
  Description of Artificial Sequence Synthetic oligonucleotide
attcgccttgcgcatggaga

SEQ ID NO: 248 - DNA, 20 bp, Artificial Sequence
  Description of Artificial Sequence Synthetic oligonucleotide
cttcacaattctctctctct

SEQ ID NO: 249 - DNA, 20 bp, Artificial Sequence
  Description of Artificial Sequence Synthetic oligonucleotide
aactggttaatcgcatcttt

SEQ ID NO: 250 - DNA, 20 bp, Artificial Sequence
  Description of Artificial Sequence Synthetic oligonucleotide
tcgaccctcgagagataaac

SEQ ID NO: 251 - DNA, 20 bp, Artificial Sequence
  Description of Artificial Sequence Synthetic oligonucleotide
gccatggtgaagggtatttt

SEQ ID NO: 252 - DNA, 20 bp, Artificial Sequence
  Description of Artificial Sequence Synthetic oligonucleotide
ggaaaaattgccatggtgaa

SEQ ID NO: 253 - DNA, 20 bp, Artificial Sequence
  Description of Artificial Sequence Synthetic oligonucleotide
cggaaaaattgccatggtga

SEQ ID NO: 254 - DNA, 20 bp, Artificial Sequence
  Description of Artificial Sequence Synthetic oligonucleotide
ccaacacccccaacatttga

SEQ ID NO: 255 - DNA, 20 bp, Artificial Sequence
  Description of Artificial Sequence Synthetic oligonucleotide
ccgccatggctacaacgatt

SEQ ID NO: 256 - DNA, 20 bp, Artificial Sequence
  Description of Artificial Sequence Synthetic oligonucleotide
gccgccatggctacaacgat

SEQ ID NO: 257 - DNA, 20 bp, Artificial Sequence
  Description of Artificial Sequence Synthetic oligonucleotide
caggcagcagacaagcccgc

SEQ ID NO: 258 - DNA, 20 bp, Artificial Sequence
  Description of Artificial Sequence Synthetic oligonucleotide
tgccaactgtttaatgcggc

SEQ ID NO: 259 - DNA, 20 bp, Artificial Sequence
  Description of Artificial Sequence Synthetic oligonucleotide
ccattgccaactgtttaatg

SEQ ID NO: 260 - DNA, 20 bp, Artificial Sequence
  Description of Artificial Sequence Synthetic oligonucleotide
cgataagtgtccccagaata

SEQ ID NO: 261 - DNA, 20 bp, Artificial Sequence
  Description of Artificial Sequence Synthetic oligonucleotide
atagaccaacccataatgcc

SEQ ID NO: 262 - DNA, 20 bp, Artificial Sequence
  Description of Artificial Sequence Synthetic oligonucleotide
accatggataacattacctg

SEQ ID NO: 263 - DNA, 20 bp, Artificial Sequence
  Description of Artificial Sequence Synthetic oligonucleotide
ttggcgttgccactgaatag

SEQ ID NO: 264 - DNA, 20 bp, Artificial Sequence
  Description of Artificial Sequence Synthetic oligonucleotide
cgctgaaaatctcgcttgct

SEQ ID NO: 265 - DNA, 20 bp, Artificial Sequence
  Description of Artificial Sequence Synthetic oligonucleotide
acatgaggggacggagaagc

SEQ ID NO: 266 - DNA, 20 bp, Artificial Sequence
  Description of Artificial Sequence Synthetic oligonucleotide
agtggtgaattttgtttgat

SEQ ID NO: 267 - DNA, 20 bp, Artificial Sequence
  Description of Artificial Sequence Synthetic oligonucleotide
tttaacggctgggtaaccag

SEQ ID NO: 268 - DNA, 20 bp, Artificial Sequence
  Description of Artificial Sequence Synthetic oligonucleotide
cgggctcgtttttaacggct

SEQ ID NO: 269 - DNA, 20 bp, Artificial Sequence
  Description of Artificial Sequence Synthetic oligonucleotide
tttctctgttagaactgagc

SEQ ID NO: 270 - DNA, 20 bp, Artificial Sequence
  Description of Artificial Sequence Synthetic oligonucleotide
tgatgtttattgtaagcctg

SEQ ID NO: 271 - DNA, 20 bp, Artificial Sequence
  Description of Artificial Sequence Synthetic oligonucleotide
atgaccaccaccacaatcaa

SEQ ID NO: 272 - DNA, 20 bp, Artificial Sequence
  Description of Artificial Sequence Synthetic oligonucleotide
tgagtcgtgttaatgcgact

SEQ ID NO: 273 - DNA, 20 bp, Artificial Sequence
  Description of Artificial Sequence Synthetic oligonucleotide
ttgcattaacctatgatcat

SEQ ID NO: 274 - DNA, 20 bp, Artificial Sequence
  Description of Artificial Sequence Synthetic oligonucleotide
tgccatttccgacgcaatcg

SEQ ID NO: 275 - DNA, 20 bp, Artificial Sequence
  Description of Artificial Sequence Synthetic oligonucleotide
aagcagcacgtctggtttta

SEQ ID NO: 276 - DNA, 20 bp, Artificial Sequence
  Description of Artificial Sequence Synthetic oligonucleotide
caaatcgttccacaatcgct

SEQ ID NO: 277 - DNA, 20 bp, Artificial Sequence
  Description of Artificial Sequence Synthetic oligonucleotide
agtttgagcgttaacatcta

SEQ ID NO: 278 - DNA, 20 bp, Artificial Sequence
  Description of Artificial Sequence Synthetic oligonucleotide
cgactggtttaacattttcg

SEQ ID NO: 279 - DNA, 20 bp, Artificial Sequence
  Description of Artificial Sequence Synthetic oligonucleotide
caaactgggttgattgagtt

SEQ ID NO: 280 - DNA, 20 bp, Artificial Sequence
  Description of Artificial Sequence Synthetic oligonucleotide
gtattatctaggtttaactt

SEQ ID NO: 281 - DNA, 20 bp, Artificial Sequence
  Description of Artificial Sequence Synthetic oligonucleotide
agtgttgggcggtattatct

SEQ ID NO: 282 - DNA, 20 bp, Artificial Sequence
  Description of Artificial Sequence Synthetic oligonucleotide
agaaagagcggagtgttggg

SEQ ID NO: 283 - DNA, 20 bp, Artificial Sequence
  Description of Artificial Sequence Synthetic oligonucleotide
acactaatgacaaaactaca

SEQ ID NO: 284 - DNA, 20 bp, Artificial Sequence
  Description of Artificial Sequence Synthetic oligonucleotide
aacactaatgacaaaactac

SEQ ID NO: 285 - DNA, 20 bp, Artificial Sequence
  Description of Artificial Sequence Synthetic oligonucleotide
aaccagtccatagcttggac

SEQ ID NO: 286 - DNA, 20 bp, Artificial Sequence
  Description of Artificial Sequence Synthetic oligonucleotide
tcaaactgaacacgtaggcg

SEQ ID NO: 287 - DNA, 20 bp, Artificial Sequence
  Description of Artificial Sequence Synthetic oligonucleotide
gtgtttcaaactgaacacgt

SEQ ID NO: 288 - DNA, 20 bp, Artificial Sequence
  Description of Artificial Sequence Synthetic oligonucleotide
gctttcctcgacccgcggct

SEQ ID NO: 289 - DNA, 20 bp, Artificial Sequence
  Description of Artificial Sequence Synthetic oligonucleotide
ttagagccagcagaaacagt

SEQ ID NO: 290 - DNA, 20 bp, Artificial Sequence
  Description of Artificial Sequence Synthetic oligonucleotide
ggttgatttcgttgtagtaa

SEQ ID NO: 291 - DNA, 20 bp, Artificial Sequence
  Description of Artificial Sequence Synthetic oligonucleotide
tgattgcttgatgttcaaac

SEQ ID NO: 292 - DNA, 20 bp, Artificial Sequence
  Description of Artificial Sequence Synthetic oligonucleotide
agtagcaaagtaactaaaat

SEQ ID NO: 293 - DNA, 20 bp, Artificial Sequence
  Description of Artificial Sequence Synthetic oligonucleotide
ggaggcgctgctatcgtgat

SEQ ID NO: 294 - DNA, 20 bp, Artificial Sequence
  Description of Artificial Sequence Synthetic oligonucleotide
acaatatatcctccggaagg

SEQ ID NO: 295 - DNA, 20 bp, Artificial Sequence
  Description of Artificial Sequence Synthetic oligonucleotide
atcacgatgctgcttttttc

SEQ ID NO: 296 - DNA, 20 bp, Artificial Sequence
  Description of Artificial Sequence Synthetic oligonucleotide
gcgtataagtgatataacgc

SEQ ID NO: 297 - DNA, 20 bp, Artificial Sequence
  Description of Artificial Sequence Synthetic oligonucleotide
tgcgtataagtgatataacg

SEQ ID NO: 298 - DNA, 20 bp, Artificial Sequence
  Description of Artificial Sequence Synthetic oligonucleotide
taatacacctttattcatgt

SEQ ID NO: 299 - DNA, 20 bp, Artificial Sequence
  Description of Artificial Sequence Synthetic oligonucleotide
ctgctttaataacaacaaat

SEQ ID NO: 300 - DNA, 20 bp, Artificial Sequence
  Description of Artificial Sequence Synthetic oligonucleotide
cgaaccgcctaatccagcag

SEQ ID NO: 301 - DNA, 20 bp, Artificial Sequence
  Description of Artificial Sequence Synthetic oligonucleotide
gttccactcaccaatgttct

SEQ ID NO: 302 - DNA, 20 bp, Artificial Sequence
  Description of Artificial Sequence Synthetic oligonucleotide
agaaaaagcggtcaacgcag

SEQ ID NO: 303 - DNA, 20 bp, Artificial Sequence
  Description of Artificial Sequence Synthetic oligonucleotide
gggaaaagagagagaaaaag

SEQ ID NO: 304 - DNA, 20 bp, Artificial Sequence
  Description of Artificial Sequence Synthetic oligonucleotide
ttccacccaacgaggaagtc

SEQ ID NO: 305 - DNA, 20 bp, Artificial Sequence
  Description of Artificial Sequence Synthetic oligonucleotide
gcgccatattccacccaacg

SEQ ID NO: 306 - DNA, 20 bp, Artificial Sequence
  Description of Artificial Sequence Synthetic oligonucleotide
attgacgcagcctgcaagaa

SEQ ID NO: 307 - DNA, 20 bp, Artificial Sequence
  Description of Artificial Sequence Synthetic oligonucleotide
gagcaacaaaattgaagagg

SEQ ID NO: 308 - DNA, 20 bp, Artificial Sequence
  Description of Artificial Sequence Synthetic oligonucleotide
aatgagcaacaaaattgaag

SEQ ID NO: 309 - DNA, 20 bp, Artificial Sequence
  Description of Artificial Sequence Synthetic oligonucleotide
tatgttcgctggtttgagct

SEQ ID NO: 310 - DNA, 20 bp, Artificial Sequence
  Description of Artificial Sequence Synthetic oligonucleotide
agtcagtagagcaataatta

SEQ ID NO: 311 - DNA, 20 bp, Artificial Sequence
  Description of Artificial Sequence Synthetic oligonucleotide
aggttgcggtgaaggttacg

SEQ ID NO: 312 - DNA, 20 bp, Artificial Sequence
  Description of Artificial Sequence Synthetic oligonucleotide
ctcatttgaggttgcggtga

SEQ ID NO: 313 - DNA, 20 bp, Artificial Sequence
  Description of Artificial Sequence Synthetic oligonucleotide
gagaattaatacttttttat

SEQ ID NO: 314 - DNA, 20 bp, Artificial Sequence
  Description of Artificial Sequence Synthetic oligonucleotide
cttctgaacgatgttgagaa

SEQ ID NO: 315 - DNA, 20 bp, Artificial Sequence
  Description of Artificial Sequence Synthetic oligonucleotide
acttctgaacgatgttgaga

SEQ ID NO: 316 - DNA, 20 bp, Artificial Sequence
  Description of Artificial Sequence Synthetic oligonucleotide
tggaatcgacctactttgag

SEQ ID NO: 317 - DNA, 20 bp, Artificial Sequence
  Description of Artificial Sequence Synthetic oligonucleotide
taacgatacggttagctaac

SEQ ID NO: 318 - DNA, 20 bp, Artificial Sequence
  Description of Artificial Sequence Synthetic oligonucleotide
tcattggcggcataacgata

SEQ ID NO: 319 - DNA, 42 bp, Artificial Sequence
  Description of Artificial Sequence Synthetic oligonucleotide
tggaaccatatggtaaagatcgtccgttgctgattggtgtaa

SEQ ID NO: 320 - protein, 14 aa, Artificial Sequence
  Description of Artificial Sequence Synthetic peptide
LEPYGKDRPLLIGV

SEQ ID NO: 321 - DNA, 42 bp, Artificial Sequence
  Description of Artificial Sequence Synthetic oligonucleotide
tggaaccatatggtaaagatcgtccgtagctgattggtgtaa

SEQ ID NO: 322 - DNA, 42 bp, Artificial Sequence; identical to SEQ ID NO: 321
  Description of Artificial Sequence Synthetic oligonucleotide

SEQ ID NO: 323 - DNA, 20 bp, Artificial Sequence
  Description of Artificial Sequence Synthetic oligonucleotide
aggttcggctaacaaggtcg

SEQ ID NO: 324 - DNA, 20 bp, Artificial Sequence
  Description of Artificial Sequence Synthetic oligonucleotide
gtctgaaggttcggctaaca

SEQ ID NO: 325 - DNA, 20 bp, Artificial Sequence
  Description of Artificial Sequence Synthetic oligonucleotide
tacaggcgagtctgaaggtt

SEQ ID NO: 326 - DNA, 20 bp, Artificial Sequence
  Description of Artificial Sequence Synthetic oligonucleotide
aacagactcagcgctcattg

SEQ ID NO: 327 - DNA, 20 bp, Artificial Sequence
  Description of Artificial Sequence Synthetic oligonucleotide
gtagtgacaagtgttggcca

(19) United States
(12) Patent Application Publication
(10) Pub. No.: US 2019/0241899 A1
(43) Pub. Date: Aug. 8, 2019

(54) Methods of Crispr Mediated Genome Modulation in V. Natriegens

(71) Applicant: President and Fellows of Harvard College, Cambridge, MA (US)

(72) Inventors: George M. Church, Brookline, MA (US); Henry Hung-yi Lee, Brookline, MA (US); Nili Ostrov, Brookline, MA (US)

(21) Appl. No.: 16/339,019

(22) PCT Filed: Oct. 5, 2017
(86) PCT No.: PCT/US17/55386

§ 371 Date: Apr. 3, 2019

Related U.S. Application Data
(60) Provisional application No. 62/455,668, filed on Feb. 7, 2017.
(60) Provisional application No. 62/404,518, filed on Oct. 5, 2016.

(51) Int. Cl. C12N 15/74

(52) U.S. Cl. CPC C12N 15/74; C12N 2795/00043

(57) ABSTRACT

Methods and compositions are provided for modulating expression of a target nucleic acid sequence within a non-E. coli cell. The method includes providing the cell with a guide RNA comprising a portion that is complementary to all or a portion of the target nucleic acid sequence, and providing the cell a Cas protein, wherein the guide RNA and the Cas protein co-localize at the target nucleic acid sequence and wherein the Cas protein modulate the expression of the target nucleic acid sequence.

### RELATED APPLICATION DATA

This application claims priority to U.S. Provisional Application No. 62/404,518 filed on Oct. 5, 2016 and U.S. Provisional Application No. 62/455,668 filed on Feb. 7, 2017 which are hereby incorporated herein by reference in its entirety for all purposes.

### STATEMENT OF GOVERNMENT INTERESTS

This invention was made with government support under Grant No. DE-FG02-02ER63445 from the United States Department of Energy. The government has certain rights in the invention.

### FIELD

The present invention relates in general to methods of genome modulation in the organism V. natrigens, such as by using CRISPR system.

### BACKGROUND

Methods of genome modulation are known and have been carried out in E. coli, S. enterica, Pseudomonas putida KT2440, Pseudomonas syringae, Pseudomonas aerginosa, Y. pseudotuberculosis, M. tuberculosis, S. cerevisiae and a growing number of organisms.

### SUMMARY

According to one aspect, the present disclosure provides a method of altering a target nucleic acid sequence within a non-E. coli cell including providing a cell with a functioning beta-like recombinase and a donor nucleic acid sequence, wherein the donor nucleic acid sequence is inserted into the target nucleic acid sequence as a result of the functioning beta-recombinase.

In one embodiment, the present disclosure provides that the non-E. coli cell is Vibrio natriegens. In one embodiment, the present disclosure provides that the beta-like recombinase is identified in a horizontal gene transfer element such as a phage. In another embodiment, the present disclosure provides that the beta-like recombinase is identified in a horizontal gene transfer element such as an Integrative and Conjugative Element (ICE). In one embodiment, the present disclosure provides that the beta-like recombinase is identified in a horizontal gene transfer element such as a conjugative plasmid. In another embodiment, the present disclosure provides that the beta-like recombinase is identified in a horizontal gene transfer element such as a Vibrio spp. phage. In yet another embodiment, the present disclosure provides that the beta-like recombinase is identified in a horizontal gene transfer element such as a Vibrio spp. Integrative and Conjugative Element (ICE). In one embodiment, the present disclosure provides that the beta-like recombinase is s065. In one embodiment, the present disclosure provides that additional recombination assisting proteins are provided to the cell. In another embodiment, the present disclosure provides that additional recombination assisting proteins are provided to the cell including the exonuclease s066, and a host nuclease inhibitor such as gam, and a single-strand DNA binding (SSB) protein s064 (Uniprot: A0A0X1L3H7). In yet another embodiment, the present disclosure provides that additional recombination assisting proteins are provided to the cell including s066, and gam to create a single-stranded intermediate from a double stranded nucleic acid donor. In one embodiment, the present disclosure provides that the donor nucleic acid sequence is introduced into the cell as a single stranded nucleic acid. In one embodiment, the present disclosure provides that the donor nucleic acid sequence is introduced into the cell as a double stranded nucleic acid. In one embodiment, the present disclosure provides that the cell has been genetically modified to include a foreign nucleic acid sequence encoding the recombinase. In another embodiment, the present disclosure provides that the cell has been genetically modified to include a foreign nucleic acid sequence encoding the recombinase, exonuclease, and host nuclease inhibitor. In yet another embodiment, the present disclosure provides that the cell has been genetically modified to include a foreign nucleic acid sequence encoding the s065, exonuclease, and host nuclease inhibitor. In still another embodiment, the present disclosure provides that the cell has been genetically modified to include a foreign nucleic acid sequence encoding the s065, s066, and host nuclease inhibitor. In one embodiment, the present disclosure provides that the cell has been genetically modified to include a foreign nucleic acid sequence encoding the s065, s066, and gam. In one embodiment, the present disclosure provides that the donor nucleic acid sequence is provided to the cell by electroporation.

According to another aspect, the present disclosure provides a method of altering a target nucleic acid sequence within a Vibrio natriegens cell including providing the Vibrio natriegens cell with a functioning beta-like recombinase and a donor nucleic acid sequence, wherein the donor nucleic acid sequence is inserted into the target nucleic acid sequence as a result of the functioning beta-recombinase.

In one embodiment, the present disclosure provides that the beta-like recombinase is identified in a horizontal gene transfer element such as a phage. In another embodiment, the present disclosure provides that the beta-like recombinase is identified in a horizontal gene transfer element such as an Integrative and Conjugative Element (ICE). In one embodiment, the present disclosure provides that the beta-like recombinase is identified in a horizontal gene transfer element such as a conjugative plasmid. In another embodiment, the present disclosure provides that the beta-like recombinase is identified in a horizontal gene transfer element such as a Vibrio spp. phage. In yet another embodiment, the present disclosure provides that the beta-like recombinase is identified in a horizontal gene transfer element such as a Vibrio spp. Integrative and Conjugative Element (ICE). In one embodiment, the present disclosure provides that the beta-like recombinase is s065. In one embodiment, the present disclosure provides that additional recombination assisting proteins are provided to the cell. In another embodiment, the present disclosure provides that additional recombination assisting proteins are provided to the cell including the exonuclease s066, a host nuclease inhibitor such as gam, and SSB. In yet another embodiment, the present disclosure provides that additional recombination assisting proteins are provided to the cell including s066, and gam to create a single-stranded intermediate from a double stranded nucleic acid donor. In one embodiment, the present disclosure provides that the donor nucleic acid sequence is introduced into the cell as a single stranded nucleic acid. In one embodiment, the present disclosure provides that the donor nucleic acid sequence is introduced into the cell as a double stranded nucleic acid. In one embodiment, the present disclosure provides that the cell has been genetically modified to include a foreign nucleic acid sequence encoding the recombinase. In another embodiment, the present disclosure provides that the cell has been genetically modified to include a foreign nucleic acid sequence encoding the recombinase, exonuclease, host nuclease inhibitor, and SSB. In yet another embodiment, the present disclosure provides that the cell has been genetically modified to include a foreign nucleic acid sequence encoding the s065, exonuclease, host nuclease inhibitor, and SSB. In still another embodiment, the present disclosure provides that the cell has been genetically modified to include a foreign nucleic acid sequence encoding the s065, s066, s064, and host nuclease inhibitor. In one embodiment, the present disclosure provides that the cell has been genetically modified to include a foreign nucleic acid sequence encoding the s065, s066, s064, and gam. In one embodiment, the present disclosure provides that the donor nucleic acid sequence is provided to the cell by electroporation.

According to another aspect, the present disclosure provides a method of altering a target nucleic acid sequence within a Vibrio natriegens cell including providing the Vibrio natriegens cell with a functioning s065 recombinase and a donor nucleic acid sequence, wherein the donor nucleic acid sequence is inserted into the target nucleic acid sequence as a result of the functioning s065.

In one embodiment, the present disclosure provides that additional recombination assisting proteins are provided to the cell. In another embodiment, the present disclosure provides that additional recombination assisting proteins are provided to the cell including the exonuclease s066, and a host nuclease inhibitor such as gam. In yet another embodiment, the present disclosure provides that additional recombination assisting proteins are provided to the cell including s066, and gam to create a single-stranded intermediate from a double stranded nucleic acid donor. In one embodiment, the present disclosure provides that the donor nucleic acid sequence is introduced into the cell as a single stranded nucleic acid. In one embodiment, the present disclosure provides that the donor nucleic acid sequence is introduced into the cell as a double stranded nucleic acid. In one embodiment, the present disclosure provides that the cell has been genetically modified to include a foreign nucleic acid sequence encoding the recombinase. In another embodiment, the present disclosure provides that the cell has been genetically modified to include a foreign nucleic acid sequence encoding the recombinase, exonuclease, and host nuclease inhibitor. In yet another embodiment, the present disclosure provides that the cell has been genetically modified to include a foreign nucleic acid sequence encoding the s065, exonuclease, host nuclease inhibitor, and SSB. In still another embodiment, the present disclosure provides that the cell has been genetically modified to include a foreign nucleic acid sequence encoding the s065, s066, s064, and host nuclease inhibitor. In one embodiment, the present disclosure provides that the cell has been genetically modified to include a foreign nucleic acid sequence encoding the s065, s066, s064, and gam. In one embodiment, the present disclosure provides that the donor nucleic acid sequence is provided to the cell by electroporation.

According to another aspect, the present disclosure provides a genetically modified Vibrio natriegens cell comprising a foreign nucleic acid sequence encoding a beta-like recombinase.

In one embodiment, the present disclosure provides that the beta-like recombinase is s065. In another embodiment, the present disclosure provides that the genetically modified Vibrio natriegens cell further includes a foreign donor nucleic acid sequence. In yet another embodiment, the present disclosure provides that the genetically modified Vibrio natriegens cell further includes a foreign donor nucleic acid sequence inserted into plasmid or genomic DNA within the Vibrio natriegens cell.

According to one aspect, the present disclosure provides a method of modulating expression of a target nucleic acid sequence within a non-E. coli cell. The method includes providing the cell with a guide RNA comprising a portion that is complementary to all or a portion of the target nucleic acid sequence, providing the cell a Cas protein, wherein the guide RNA and the Cas protein co-localize at the target nucleic acid sequence and wherein the Cas protein modulate the expression of the target nucleic acid sequence.

According to another aspect, the present disclosure provides a method of altering a target nucleic acid sequence within a non-E. coli cell. The method include providing the cell with a guide RNA comprising a portion that is complementary to all or a portion of the target nucleic acid sequence, providing the cell a Cas protein, and providing the cell a donor nucleic acid sequence, wherein the guide RNA and the Cas protein co-localize at the target nucleic acid sequence, wherein the Cas protein cleaves the target nucleic acid sequence and the donor nucleic acid sequence is inserted into the target nucleic acid sequence in a site specific manner.

In some embodiments, the non-E. coli cell is Vibrio natriegens. In some embodiments, the Cas protein is a Cas9 protein. In other embodiments, the Cas9 is a Cas9 nickase or a nuclease null Cas9 (dCas9). In some embodiments, the Cas9 is further fused with a transcription repressor or activator. In other embodiments, the guide RNA and/or Cas protein are provided on a vector. In one embodiment, the vector is a plasmid. In some embodiments, a plurality of guide RNAs that are complementary to different target nucleic acid sequences are provided to the cell and wherein expressions of different target nucleic acid sequences are modulated. In certain embodiments, expression of Cas protein is inducible. In some embodiments, the cell has been genetically modified to include a foreign nucleic acid sequence. In some embodiments, the foreign nucleic acid sequence encodes a reporter protein. In one embodiment, the reporter protein is GFP. In some embodiments, the providing step comprising providing nucleic acid sequences encoding the guide RNA and/or the Cas protein to the cell by transfection or electroporation. In other embodiments, the guide RNA, Cas protein and donor nucleic acid sequence are provided on a vector. In one embodiment, the vector is a plasmid. In some embodiments, the guide RNA, Cas protein and donor nucleic acid sequence are provided on plasmids and provided to the cell by electroporation. In some embodiments, the donor nucleic acid sequence is introduced into the cell as a single stranded nucleic acid. In other embodiments, the donor nucleic acid sequence is introduced into the cell as a double stranded nucleic acid.

According to another aspect, the present disclosure provides a nucleic acid construct. In one embodiment, the nucleic acid construct encodes a guide RNA comprising a portion that is complementary to a target nucleic acid sequence in Vibrio natriegens. In another embodiment, the nucleic acid construct encodes a Cas protein. In yet another embodiment, the nucleic acid construct encodes a donor nucleic acid sequence for insertion into a target nucleic acid sequence in Vibrio natriegens.

According to another aspect, the present disclosure provides a non-E. coli cell. In one embodiment, the cell comprises a guide RNA comprising a portion that is complementary to all or a portion of the target nucleic acid sequence, and a Cas protein, wherein the guide RNA and the Cas protein co-localize at the target nucleic acid sequence and modulates the expression of the target nucleic acid sequence in the cell. In another embodiment, the cell comprises a guide RNA comprising a portion that is complementary to all or a portion of the target nucleic acid sequence, a Cas protein, and a donor nucleic acid sequence, wherein the guide RNA and the Cas protein co-localize at the target nucleic acid sequence, wherein the Cas protein cleaves the target nucleic acid sequence and the donor nucleic acid sequence is inserted into the target nucleic acid sequence in a site specific manner. In one embodiment, the non-E. coli cell is Vibrio natriegens.

According to one aspect, the present disclosure provides a method of improving the growth rate of a non-E. coli cell by suppressing the expression of a target gene of the non-E. coli cell. In certain embodiments, a plurality of target gene expression is suppressed. In one embodiment, the expression of the target gene is suppressed by transcriptional repression. In another embodiment, the expression of the target gene is suppressed by mutagenization of the target gene. In yet another embodiment, the expression of the target gene is suppressed by providing the cell with a guide RNA comprising a portion that is complementary to all or a portion of a gene sequence, and providing the cell a Cas protein, wherein the guide RNA and the Cas protein co-localize at the gene sequence and suppress the target gene expression. In one embodiment, the non-E. coli cell is Vibrio natriegens. In one embodiment, the Cas protein is a Cas9 protein. In other embodiments, the Cas9 is a Cas9 nickase or a nuclease null Cas9 (dCas9). In certain embodiment, the Cas9 is further fused with a transcription repressor. In one embodiment, the guide RNA and Cas protein are each provided to the cell via a vector comprising nucleic acid encoding the guide RNA and the Cas protein. In one embodiment, the vector is a plasmid. In some embodiments, a plurality of guide RNAs that are complementary to different gene sequences are provided to the cell and wherein expressions of different target genes are suppressed. In certain embodiments expression of Cas protein is inducible. In one embodiment, the providing step comprising providing nucleic acid sequences encoding the guide RNA and the Cas protein to the cell by transfection or electroporation. In some embodiments, the target gene comprises genes in Table 3. In other embodiments, the target gene comprises ATP-dependent DNA helicase RecQ, N-acyl-L-amino acid amidohydrolase, a hypothetical protein fused to ribosomal protein S6 glutaminyl transferase, ABC transporter2C periplasmic spermidine putrescine-binding protein PotD, a putative protease, Na+/H+ antiporter NhaP, methyl-accepting chemotaxis protein, transporter2C putative, biotin synthesis protein BioC, alkaline serine protease, glutamate aspartate transport system permease protein GltJ, thiamin ABC transporter2C transmembrane component, or putrescine utilization regulator. In some embodiments, the guide RNA includes complementary sequences in Table 4 for use in target gene suppression.

Further features and advantages of certain embodiments of the present disclosure will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 is a graph depicting data regarding resistant colonies as a result of recombineering of single-stranded oligonucleotides in V. natriegens using λ-Beta and SXT s065. The single-stranded oligonucleotide reverts a spectinomycin with a premature stop codon into a functional spectinomycin gene on plasmid.

FIG. 2 is a graph depicting data regarding resistant colonies as a result of recombineering with s065 and oligonucleotides targeting the forward (leading strand) or reverse (lagging strand) of DNA replication. A single-stranded oligonucleotide recombines with a spectinomycin gene, on a plasmid, with a premature stop codon to convert it into a functional spectinomycin gene.

FIG. 3 is a graph depicting data regarding resistant colonies as a result of recombineering based on the amount of oligonucleotide where an increased oligo amount used for s065-mediated recombination in V. natriegens. A single-stranded oligonucleotide recombines with a spectinomycin gene, on a plasmid, with a premature stop codon to convert it into a functional spectinomycin gene.

FIG. 4 is a graph depicting data regarding resistant colonies as a result of recombineering based on the number of phosphorothioates on the oligonucleotide added to enhance stability of the oligonucleotides in vivo. A single-stranded oligonucleotide recombines with a spectinomycin gene, on a plasmid, with a premature stop codon to convert it into a functional spectinomycin gene.

FIG. 5 depicts results of recombination on a chromosome and information as a result of Sanger sequencing of V. natriegens pyrF mutant colonies isolated by 5-FOA selection following ssDNA oligonucleotide recombination. The single-stranded oligonucleotide introduces a premature stop codon into the chromosomally encoded pyrF gene.

FIG. 6 depicts results of gene deletion by insertion of a double-stranded DNA cassette carrying an antibiotic marker with flanking homology arms into the V. natriegens genome using proteins s065 and s066 from SXT.

FIGS. 7A-7B depict results of titration of Vibrio natriegens induction systems. FIG. 7A depicts the result of induction of the lactose promoter by IPTG. FIG. 7B depicts the result of induction of the arabinose promoter by Larabinose. Data are shown as mean±SD (N≥3).

FIG. 8 depicts the result of targeted gene inhibition of chromosomally integrated GFP in Vibrio natriegens using dCas9 according to an embodiment of the present disclosure. Guide RNA (gRNA) were designed to target the template or nontemplate strand of GFP. Data are shown as mean±SD (N≥3).

FIG. 9 is a graph depicting the temperature at which electroporation of plasmids in V. natriegens is performed. “Cold” temperature is 4° C. for electroporation. “Room temperature” is 25° C. for electroporation.

FIGS. 10A-C depict quantifying V. natriegens generation time in rich and glucose-supplemented minimal media across a broad range of temperatures. FIG. 10A depicts bulk growth measurements of V. natriegens and E. coli across various temperatures (in LB3 and LB, respectively). M9 for V. natriegens was supplemented with 2% (w/v) NaCl. Glucose (0.4% w/v final) was used as a carbon source. Data shown are mean±SD (N=24). FIG. 10B depicts single-cell growth rate measurement based on conditions FIG. 10C. Data shown are mean±SD (N≥12). FIG. 10C depicts representative time course images of V. natriegens (top, LB3 media) and E. coli (bottom, LB media) growing at 37° C. for 93 minutes. Images were taken at 100× magnification.

FIGS. 11A-B depict V. natriegens genome and replication dynamics. FIG. 11A depicts two circular chromosomes are depicted. From outside inward: two outer circles represent protein-coding genes on the plus and minus strand, respectively, color coded by RAST annotation. The third circle represents G+C content relative to mean G+C content of the respective chromosome, using a sliding window of 3,000 bp. tRNA and rRNA genes are shown in the fourth and fifth circles, respectively. Below, the percentage of each RAST category relative to all annotated genes. FIG. 11B depicts filtered sequence coverage (black) and GC-skew (green) for each chromosome, as measured for exponentially growing V. natriegens in LB3 at 37° C. Origin (red) and terminus (blue) are denoted.

FIGS. 12A-G depict fitness profiling of all protein-coding genes in V. natriegens by CRISPRi. FIG. 12A depicts schematics of pooled CRISPRi screen. Distribution of relative fitness (RF) is shown for passage one and passage three of competitively grown cultures (gray, dCas9 with guides; white, guides only). FIG. 12B depicts relative fitness of V. natriegens genes after passage three. Genes that are essential for fast growth (1070 genes total) are highlighted: essentials (purple, 604 genes, RF≤0.529, p≤0.001, non-parametric) are determined after passage one. Genes specifically required for fast growth (gold, 466 genes, RF≤0.781, p≤0.05, non-parametric) are determined after passage three. FIG. 12C depicts relative fitness of V. natriegens genes after passage one. Ribosomal genes (black). Essential genes denoted by dotted boxed region. FIG. 12D depicts overlap of putative essential V. natriegens genes with essentials found in E. coli and V. cholerae. FIG. 12E depicts relative fitness of ribosomal proteins, in the absence (open circles) or presence of V. natriegens expressing dCas9 (closed circles). Filled grey square indicates essentiality in V. natriegens (Vn, current study), V. cholerae (Vc) or E. coli (Ec). FIG. 12F depicts RAST categories for essential and fast growth gene sets. Number of essential (purple) and fast growing (gold) genes are shown out of all annotated V. natriegens genes (white). Asterisks indicates statistical enrichment (p<0.05, BH-adjusted). Fold increase in each RAST category between fast growth subset and essentials (black circles). FIG. 12G depicts spatial distribution of essential genes (outer circle, purple) and genes required for fast growth (inner circle, gold) on V. natriegens chromosomes.

FIG. 13 depicts plasmid transformation in V. natriegens. Bright field (left) and fluorescence images (right) of V. natriegens colonies transformed with plasmids carrying the following replicons (a) colEl (b) SC101 (c) RSF1010. All plasmid carry constitutive GFP expression cassette pLtetO-GFP.

FIGS. 14A-G depict optimization of DNA transformation. FIG. 14A depicts cell viability in sorbitol, used as an osmoprotectant (representative data). Transformation efficiencies were optimized for the following criteria: (FIG. 14B) Voltage. (FIG. 14C) Recovery media. (FIG. 14D) Amount of input plasmid DNA. (FIG. 14E) Recovery time. (FIG. 14F) competent cell storage: transformation efficiencies of electrocompetent cells stored at −80° C. over time. (day 0: freshly prepared electrocompetent cells). Unless otherwise indicated, transformations were performed using 50 ng plasmid DNA with recovery time of 45 min at 37° C. in SOC3 media. Data are shown as mean±SD (N≥2). FIG. 14G depicts rapid DNA amplification in V. natriegens. Single colonies of V. natriegens or E. coli were used to inoculate 3 mL liquid LB3 or LB, respectively. Cultures were grown for 5 hours at 37° C. and plasmid DNA was extracted and quantified. Data are shown as mean±SD (N≥3).

FIGS. 15A-C depict CTX bacteriophage replication and infectivity. FIG. 15A depicts V. natriegens transformants of CTX-Km RF (left) and recombinant vector, pRST, carrying the replicative CTX origin (right). FIG. 15B depicts transduction of V. natriegens (left) and V. cholerae 0395 (right) by CTX-Km^{Vc}Φ bacteriophage produced by V. cholerae 0395. FIG. 15C depicts transduction of V. natriegens (left) and V. cholerae 0395 (right) by CTX-Km^{Vn}Φ bacteriophage produced by V. natriegens.

FIGS. 16A-C depict establishing CRISPR/Cas9 functionality in V. natriegens. FIG. 16A depicts nuclease activity of Cas9. Guide-dependent lethality was observed upon cutting of chromosomal targets. Data are shown as mean±SD (N?3). Colonies of V. natriegens with chromosomal integration of GFP were not detected (N.D.) when Cas9 and a GFP-targeting guide was coexpressed. FIG. 16B depicts dCas9 inhibition of chromosomally-integrated GFP. Guide RNAs (gRNAs) were designed to target the template (T) or non-template (NT) strand of GFP proximal to the transcriptional start site. Data are shown as mean±SD (N≥_3). Greater inhibition observed when targeting the non-template (NT, >13-fold) over template (T, 3.7-fold) strand, in line with previous reports (Qi, L. S. et al. Repurposing CRISPR as an RNA-guided platform for sequence-specific control of gene expression. Cell 152, 1173-1183 (2013)). Significant GFP repression was observed without induction, indicating basal expression of dCas9. To maximize consistency in subsequent experiments, further experiments thus used the presence and absence of dCas9 in lieu of induction. FIG. 16C depicts a small scale pooled CRISPRi screen. CRISPRi assay in wild-type V. natriegens expressing dCas9 was performed by co-targeting five genes: growth-neutral genes (flgCv, flagellar subunit and two for GFP), putative essential genes (lptF_{Vn}, an essential gene in E. coli critical for the lipopolysaccharide transport system), and a negative control (the E. coli sequence for gene lptF_{Ec}). The pooled cell library was grown as a single batch culture under competitive growth conditions at 37° C., and gRNA abundance was quantified by sequencing at several time points. Fold change for each target is computed as the normalized gRNA abundance to reads per million and expressed as a ratio relative to initial conditions. Depletion was only observed for the putative essential V. natriegens gene (lptF_{Vn}), demonstrating specificity and sensitivity of this pooled screen. These data establishes CRISPR in V. natriegens and illustrates the utility of a pooled CRISPRi screen.

FIG. 17 depicts distribution of relative fitness scores for all V. natriegens protein-coding genes, as generated by pooled CRISPRi screen. Control (-dCas9) shown in green, inhibition assay (+dCas9) shown in blue. Data shown for three serial passages.

FIG. 18 depicts growth rates of various V. natriegens. The figure shows the time in minutes it takes for various strains to grow to exponential phase (optical density measured at 600 nm of ˜0.2).

### DETAILED DESCRIPTION

Aspects of the present disclosure are directed to recombineering methods in non-E. coli microbes, such as Vibrio natriegens. Aspects of the present disclosure are directed to the use of one or more recombinases for recombineering methods in non-E. coli microbes, such as Vibrio natriegens. Aspects of the present disclosure utilize recombineering materials and methods known to those of skill in the art. Recombineering or recombination-mediated genetic engineering is a genetic and molecular biology technique that utilizes the recombination system of a cell, such as homologous recombination. Materials and methods useful for recombineering are described in Ellis, H. M., D. Yu, T. DiTizio & D. L. Court, (2001) High efficiency mutagenesis, repair, and engineering of chromosomal DNA using single-stranded oligonucleotides. Proc. Natl. Acad. Sci. USA 98: 6742-6746; Lajoie, M. J. et al., 2013. Genomically recoded organisms expand biological functions. Science, 342(6156), pp. 357-360; Wang, H. H. et al. Programming cells by multiplex genome engineering and accelerated evolution. Nature 460, 894-898 (2009); Thomason, L. C. et al., 2014. Recombineering: genetic engineering in bacteria using homologous recombination. Current protocols in molecular biology/edited by Frederick M. Ausubel . . . [et al.], 106, pp. 1.16.1-39; Hmelo, L. R. et al., 2015. Precision-engineering the Pseudomonas aeruginosa genome with two-step allelic exchange. Nature protocols, 10(11), pp. 1820-1841; Luo, X. et al., 2016. Pseudomonas putida KT2440 markerless gene deletion using a combination of λ Red recombineering and Cre/loxP site-specific recombination. FEMS microbiology letters, 363(4). Available at: http://dx.doi.org/10.1093/femsle/fnw014; and Swingle, B. et al., 2010. Recombineering Using RecTE from Pseudomonas syringae. Applied and environmental microbiology, 76(15), pp. 4960-4968 each of which are hereby incorporated by reference in its entirety.

In E. coli, expression of λ Red Beta (also referred to as β or bet), a recombinase protein found on the λ-phage genome, potentiates recombineering by ˜10,000-fold as described in Yu, D. et al., 2000. An efficient recombination system for chromosome engineering in Escherichia coli. Proceedings of the National Academy of Sciences of the United States of America, 97(11), pp. 5978-5983 hereby incorporated by reference in its entirety. Aspects of the present disclosure are directed to the identification and use of recombinases that can be used in non-E. coli organisms, such V. natriegens.

According to one aspect, recombinases may be identified for their ability to function in a recombineering method. Exemplary recombinases include those known as s065. See Chen et al., BMC Molecular Biology (2011) 12:16 hereby incorporated by reference in its entirety. The SXT mobile genetic element was originally isolated from an emerging epidemic strain of Vibrio cholerae (serogroup O139), which causes the severe diarrheal disease cholera. Formerly referred to as a conjugative transposon, SXT is now classified as being a type of integrating conjugative element (ICE). The SXT genome contains three consecutive coding DNA sequences (CDSs; s064, s065 and s066) arranged in an operon-like structure, which encode homologues of ‘phage-like’ proteins involved in DNA recombination. The encoded S064 protein (SXT-Ssb) is highly homologous to bacterial single strand DNA (ssDNA) binding proteins (Ssb); S065 (SXT-Bet) is homologous to the Bet single stranded annealing protein (SSAP) from bacteriophage lambda (lambda-Bet, which is also referred to as a DNA synaptase or recombinase); and S066 (SXT-Exo) shares homology with the lambda Exo/YqaJ family of alkaline exonucleases.

Aspects of the present disclosure are directed to the use of one or more recombinases to promote DNA recombination within V. natriegens. According to one aspect, exemplary recombinases include s065, beta (lambda) which is the alkaline exonuclease from bacteriophage lambda, which themselves are capable of promoting single-stranded DNA recombination with oligonucleotides. According to one aspect, exemplary helper proteins include s066, exo (lambda), an exonuclease from bacteriophage lambda, and gam (lambda), a host-nuclease inhibitor protein from bacteriophage lambda, as well as single-strand DNA binding protein such as s064 which are required for stabilization and recombination of single and double-stranded DNA. Aspects of the present disclosure are directed to methods of using s065, beta (lambda) or lambda recombinases, s066, s064, and gam to promote genetic recombination of the V. natriegens genomic DNA, i.e. between single stranded oligonucleotides and the V. natriegens genomic DNA, i.e. chromosomal DNA.

Vibrio natriegens (previously Pseudomonas natriegens and Beneckea natriegens) is a Gram negative, nonpathogenic marine bacterium isolated from salt marshes. It is purported to be one of the fastest growing organisms known with a generation time between 7 to 10 minutes. According to one aspect, Vibrio natriegens is characterized, cultured and utilized for genetic engineering methods as described in bioRxiv (Jun. 12, 2016) doi: http://dx.doi.org/10.1101/058487 hereby incorporated by reference in its entirety. Vibrio natriegens includes two chromosomes of 3,248,023 bp and 1,927,310 bp that together encode 4,578 open reading frames. Vibrio natriegens may be genetically modified using tranformation protocols and compatible plasmids, such as a plasmid based on the RSF1010 operon, or a phage such as vibriophage CTX. Transformation of Vibrio natriegens with the CTX-Km RF yielded transformants which suggests that the CTX replicon is compatible in this host. A new plasmid, pRST, was constructed by fusing the specific replication genes from CTX-Km RF to a Escherichia coli plasmid based on the conditionally replicating R6k origin, thus adding a lowcopy shuttle vector to the list of available genetic tools for Vibrio natriegens. This plasmid may be used in combination with the pRSF plasmid as a dual plasmid system in Vibrio natriegens for complex regulation of proteins and high-throughput manipulation of diverse DNA libraries.

Aspects of the present disclosure are directed to methods of recombineering in non-E. coli organisms, such as V. natriegens using beta-like recombinases. An exemplary beta-like recombinases is s065 from the SXT mobile element found in Vibrio cholerae. See Beaber, J. W., Hochhut, B. & Waldor, M. K., 2002. Genomic and functional analyses of SXT, an integrating antibiotic resistance gene transfer element derived from Vibrio cholerae. Journal of bacteriology, 184(15), pp. 4259-4269 hereby incorporated by reference in its entirety.

Aspects of the present disclosure are directed to recombineering methods using linear DNA substrates that are either double-stranded (dsDNA) or single-stranded (ssDNA). Aspects of the present disclosure are directed to recombineering methods using a double-stranded DNA (dsDNA) cassette. Aspects of the present disclosure are directed to methods as described herein of recombineering of plasmid borne DNA with single stranded oligonucleotides. Aspects of the present disclosure are directed to methods as described herein of recombineering of chromosomal DNA with single stranded oligonucleotides. Aspects of the present disclosure are directed to methods as described herein of recombineering of chromosomal DNA with a double-stranded DNA cassette. According to certain aspects, s065 is used as a recombinase in the recombineering methods. According to certain aspects, Vibrio natriegens is used as the organism or cell. According to certain aspects, the methods may include the use of other components, proteins or enzymes in a recombineering system, expressed from their respective genes or otherwise provided such as s066 from SXT with or without the protein gam expressed from λ-phage.

Aspects of the present disclosure are directed to recombineering methods used to create gene replacements, deletions, insertions, and inversions, as well as, gene cloning and gene/protein tagging (His-tags etc.) For gene replacements or deletions, aspects may utilize a cassette encoding a drug-resistance gene, such as one that is made by PCR using bi-partite primers. These primers consist of (from 5′-*3′) 50 bases of homology to the target region, where the cassette is to be inserted, followed by 20 bases to prime the drug resistant cassette. The exact junction sequence of the final construct is determined by primer design. Methods to provide a cell with a nucleic acid, whether single stranded or double stranded or other genetic element are known to those of skill in the art and include electroporation. Selection and counterselection techniques are known to those of skill in the art.

The present disclosure provides methods of recombineering to perform knock-out and knock-in of genes in V. natriegens to create mutants with desired characteristics. For example, deletion of genes that catabolize DNA result in V. natriegens mutants that have improved plasmid yield and stability as described in Weinstock, M. T. et al., 2016. Vibrio natriegens as a fast-growing host for molecular biology. Nature methods, 13(10), pp. 849-851 hereby incorporated by refernece in its entirety.

The present disclosure provides methods of performing multiplex oligo recombination (MAGE or multiplex automated genome engineering as is known in the art) using recombineering for accelerated evolution in V. natriegens as described in Wang, H. H. et al., 2009. Programming cells by multiplex genome engineering and accelerated evolution. Nature, 460(7257), pp. 894-898 hereby incorporated by reference in its entirety.

The present disclosure provides methods for using recombineering to optimize metabolic pathways as described in Wang, H. H. et al., 2009. Programming cells by multiplex genome engineering and accelerated evolution. Nature, 460(7257), pp. 894-898 hereby incorporated by reference in its entirety.

The present disclosure provides methods for using recombineering to recode V. natriegens genome for virus resistance, incorporation of nonstandard amino acids, and genetic isolation as described in Ma, N.J. & Isaacs, F. J., 2016. Genomic Recoding Broadly Obstructs the Propagation of Horizontally Transferred Genetic Elements. Cell systems, 3(2), pp. 199-207; Ostrov, N. et al., 2016. Design, synthesis, and testing toward a 57-codon genome. Science, 353(6301), pp. 819-822; and Lajoie, M. J. et al., 2013. Genomically recoded organisms expand biological functions. Science, 342(6156), pp. 357-360 each of which are hereby incorporated by reference in its entirety.

According to certain aspects, recombineering components or proteins for carrying out recombineering methods in V. natriegens as described herein may be provided on a plasmid (trans) or integrated into the chromosome (cis) to create a variety of recombineering V. natriegens strains, such as those found for recombineering E. coli strains as described in world wide website redrecombineering.ncifcrf.gov/strains—plasmids.html.

Recombineering methods as described herein may be carried out using a basic protocol of growing cultures or cells such as by overnight culturing; subculturing cells in desired growth media; inducing production of recombinase within the cell or cells or providing the cell or cells with a recombinase; and introducing the single strand DNA or double strand DNA into the cell or cells, whereby the recombinase promotes recombination of the single-stranded DNA or double-stranded DNA into target DNA within the cell or cells.

According to current understanding of the recombinase mediated recombination as herein described, Beta binds single-stranded DNA (ssDNA) donor and single-stranded binding proteins in the host to facilitate homing of the single-stranded DNA donor to its homologous region in the target DNA. This single-stranded DNA donor anneals as an Okazaki fragment of DNA replication, and is incorporated into the genome during cell replication. According to the present disclosure, Beta is a phage protein and its natural function is to operate during phage (vs. bacterial) replication. When lambda phages infect a cell, they insert linear DNA, and in lytic replication this DNA is then circularized and replicated as a circular genome (first as theta-replication, then through rolling-circle). In order to make the circular form, the linear DNA from the initial insertion (and from cut concatemers from rolling-circle) have a repeated “cos” sequence at each end, and these sequences are rendered single stranded so that the two ends can hybridize and form a circle (“cos” ends=“cohesive” ends). Without intending to be bound by scientific theory, Beta may operate to help anneal these single-stranded cos ends. In recombineering, this capacity of Beta is used in a non-natural context—to help anneal oligos to the lagging strand during bacterial replication. (See, Thomason, L. C. et al., 2014, Recombineering: genetic engineering in bacteria using homologous recombination, Current protocols in molecular biology/edited by Frederick M. Ausubel . . . [et al.], 106, pp. 1.16.1-39;

Sharan, S. K. et al., 2009, Recombineering: a homologous recombination-based method of genetic engineering, Nature protocols, 4(2), pp. 206-223; Hirano, N. et al., 2011, Site-specific recombinases as tools for heterologous gene integration, Applied microbiology and biotechnology, 92(2), pp. 227-239; Mosberg, J. A., Lajoie, M. J. & Church, G. M., 2010, Lambda red recombineering in Escherichia coli occurs through a fully single-stranded intermediate, Genetics, 186(3), pp. 791-799, hereby incorporated by reference in their entireties).

According to the present disclosure, it is shown that s065 performs better than Beta for recombination in Vibrio natriegens. Recombination can also be performed with a double-stranded DNA donor, as detailed herein. This requires at least one additional protein, Exo, which is thought to digest the double-stranded DNA into a single-strand which recombines as detailed herein. Expression of the protein, Gam, inhibits endogenous digestion of this donor DNA. According to the present disclosure, it is shown that s066 and gam, in addition to s065, mediate the double-stranded recombination. Without intending to be bound by scientific theory, the improved performance of s065 is likely due to its molecular interactions with the single-stranded binding proteins in Vibrio natriegens. The fast growth rate is an attractive feature of working with Vibrio natriegens, but likely not directly responsible for s065 recombination.

According to the present disclosure, s065 is for single-stranded DNA recombination in V. natriegens for both DNA on plasmids and DNA on the chromosome. According the present disclosure, optimizing the single-stranded DNA oligos in the following way improves recombination with s065: a. the oligos are 90 base pairs long, b. the oligos target the lagging strand of DNA replication, c. the oligos are added at >100 uM for electroporation, and d. the oligos are protected by multiple phosphorothioate bonds.

According to the present disclosure, s066+gam (in addition to s065) is for double-stranded DNA recombination. The double-stranded DNA is protected by phosphorothioates at one or both 5′ ends. (See, J. A. Mosberg, M. J. Lajoie, G. M. Church, Lambda Red Recombineering in Escherichia coli Occurs Through a Fully Single-Stranded Intermediate, Genetics, Nov. 1, 2010 vol. 186 no. 3, 791-799, hereby incorporated by reference in its entirety).

According to one exemplary aspect, electrocuvettes are provided with up to 5 uL of DNA (>=50 uM of single-stranded DNA oligo and about 1 ug of double-stranded DNA oligo with 500 bp homology arms) and are placed on ice. Cells are washed in 1M cold sucrose or sorbitol, and cells are concentrated 200× by volume. Electroporation is carried out with the following settings: 0.4 kV, 1kΩ, 25 uF; time constants may be >12 ms, The cells are recovered from the electrocuvette in rich media. The cells are plated and incubated for colony formation.

### CAS9 Description

RNA guided DNA binding proteins are readily known to those of skill in the art to bind to DNA for various purposes. Such DNA binding proteins may be naturally occurring.

DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety.

In general, bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February, 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June, 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February, 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June, 2009) hereby incorporated by refernece in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January, 2011) each of which are hereby incorporated by reference in their entireties.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is provided in Deltcheva et al., Nature 471, 602-607 (2011) hereby incorporated by reference in its entirety.

Modification to the Cas9 protein is contemplated by the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase. An exemplary DNA binding protein is an RNA guided DNA binding protein nuclease of a Type II CRISPR System, such as a Cas9 protein or modified Cas9 or homolog of Cas9. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null or nuclease deficient Cas9 protein.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinek et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 (“Cas9Nuc”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. thermophiles or S. pyogenes and protein sequences having at least 30%, 40%, 50%, 60%, 70%, 80%, 90%, 95%, 98% or 99% homology thereto and being a DNA binding protein, such as an RNA guided DNA binding protein.

An exemplary CRISPR system includes the S. thermophiles Cas9 nuclease (ST1 Cas9) (see Esvelt K M, et al., Orthogonal Cas9 proteins for RNA-guided gene regulation and editing, Nature Methods., (2013) hereby incorporated by reference in its entirety). An exemplary CRISPR system includes the S. pyogenes Cas9 nuclease (Sp. Cas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). According to certain aspects, a nuclease null or nuclease deficient Cas 9 can be used in the methods described herein. Such nuclease null or nuclease deficient Cas9 proteins are described in Gilbert, L. A. et al. CRISPR-mediated modular RNA-guided regulation of transcription in eukaryotes. Cell 154, 442-451 (2013); Mali, P. et al. CAS9 transcriptional activators for target specificity screening and paired nickases for cooperative genome engineering. Nature biotechnology 31, 833-838 (2013); Maeder, M. L. et al. CRISPR RNA-guided activation of endogenous human genes. Nature methods 10, 977-979 (2013); and Perez-Pinera, P. et al. RNA-guided gene activation by CRISPR-Cas9-based transcription factors. Nature methods 10, 973-976 (2013) each of which are hereby incorporated by reference in its entirety. The DNA locus targeted by Cas9 (and by its nuclease-deficient mutant, “dCas9” precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 or a nuclease deficient Cas9 to a target nucleic acid. In a multitude of CRISPR-based biotechnology applications (see Mali, P., Esvelt, K. M. & Church, G. M. Cas9 as a versatile tool for engineering biology. Nature methods 10, 957-963 (2013); Hsu, P. D., Lander, E. S. & Zhang, F. Development and Applications of CRISPR-Cas9 for Genome Engineering. Cell 157, 1262-1278 (2014); Chen, B. et al. Dynamic imaging of genomic loci in living human cells by an optimized CRISPR/Cas system. Cell 155, 1479-1491 (2013); Shalem, O. et al. Genome-scale CRISPR-Cas9 knockout screening in human cells. Science 343, 84-87 (2014); Wang, T., Wei, J. J., Sabatini, D. M. & Lander, E. S. Genetic screens in human cells using the CRISPR-Cas9 system. Science 343, 80-84 (2014); Nissim, L., Perli, S. D., Fridkin, A., Perez-Pinera, P. & Lu, T. K. Multiplexed and Programmable Regulation of Gene Networks with an Integrated RNA and CRISPR/Cas Toolkit in Human Cells. Molecular cell 54, 698-710 (2014); Ryan, O. W. et al. Selection of chromosomal DNA libraries using a multiplex CRISPR system. eLife 3 (2014); Gilbert, L. A. et al. Genome-Scale CRISPR-Mediated Control of Gene Repression and Activation. Cell (2014); and Citorik, R. J., Mimee, M. & Lu, T. K. Sequence-specific antimicrobials using efficiently delivered RNA-guided nucleases. Nature biotechnology (2014) each of which are hereby incorporated by reference in its entirety), the guide is often presented in a so-called sgRNA (single guide RNA), wherein the two natural Cas9 RNA cofactors (gRNA and tracrRNA) are fused via an engineered loop or linker.

According to one aspect, the Cas9 protein is an enzymatically active Cas9 protein, a Cas9 protein wild-type protein, a Cas9 protein nickase or a nuclease null or nuclease deficient Cas9 protein. Additional exemplary Cas9 proteins include Cas9 proteins attached to, bound to or fused with functional proteins such as transcriptional regulators, such as transcriptional activators or repressors, a Fok-domain, such as Fok 1, an aptamer, a binding protein, PP7, MS2 and the like.

According to certain aspects, the Cas9 protein may be delivered directly to a cell by methods known to those of skill in the art, including injection or lipofection, or as translated from its cognate mRNA, or transcribed from its cognate DNA into mRNA (and thereafter translated into protein). Cas9 DNA and mRNA may be themselves introduced into cells through electroporation, transient and stable transfection (including lipofection) and viral transduction or other methods known to those of skill in the art. The Cas9 protein complexed with the guide RNA, known as a ribonucleotide protein (RNP) complex, may also be introduced to the cells via electroporation, injection, or lipofection.

Guide RNA Description

Embodiments of the present disclosure are directed to the use of a CRISPR/Cas system and, in particular, a guide RNA which may include one or more of a spacer sequence, a tracr mate sequence and a tracr sequence. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

According to certain aspects, the guide RNA is between about 10 to about 500 nucleotides. According to one aspect, the guide RNA is between about 20 to about 100 nucleotides. According to certain aspects, the spacer sequence is between about 10 and about 500 nucleotides in length. According to certain aspects, the tracr mate sequence is between about 10 and about 500 nucleotides in length. According to certain aspects, the tracr sequence is between about 10 and about 100 nucleotides in length. According to certain aspects, the linker nucleic acid sequence is between about 10 and about 100 nucleotides in length.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence (if present). According to this aspect, all of the ranges for the spacer sequence, tracr mate sequence, tracr sequence, and linker sequence (if present) are incorporated herein by reference and need not be repeated. A guide RNA as described herein may have a total length based on summing values provided by the ranges described herein. Aspects of the present disclosure are directed to methods of making such guide RNAs as described herein by expressing constructs encoding such guide RNA using promoters and terminators and optionally other genetic elements as described herein.

According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

### Donor Description

The term “donor nucleic acid” include a nucleic acid sequence which is to be inserted into genomic DNA according to methods described herein. The donor nucleic acid sequence may be expressed by the cell.

According to one aspect, the donor nucleic acid is exogenous to the cell. According to one aspect, the donor nucleic acid is foreign to the cell. According to one aspect, the donor nucleic acid is non-naturally occurring within the cell.

### Foreign Nucleic Acids Description

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

### Cells

Cells according to the present disclosure include any cell into which foreign nucleic acids can be introduced and expressed as described herein. It is to be understood that the basic concepts of the present disclosure described herein are not limited by cell type. In some embodiments, the cell is a eukaryotic cell or prokaryotic cell. In some embodiments, the prokaryotic cell is a non-E. coli cell. In an exemplary embodiment, the non-E. coli cell is Vibrio natriegens.

### Vectors

Vectors are contemplated for use with the methods and constructs described herein.

The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

### Regulatory Elements and Terminators and Tags

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 3-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure, figures and accompanying claims.

### Example I

### Recombination of Plasmid-Borne DNA with Single-Stranded Oligos

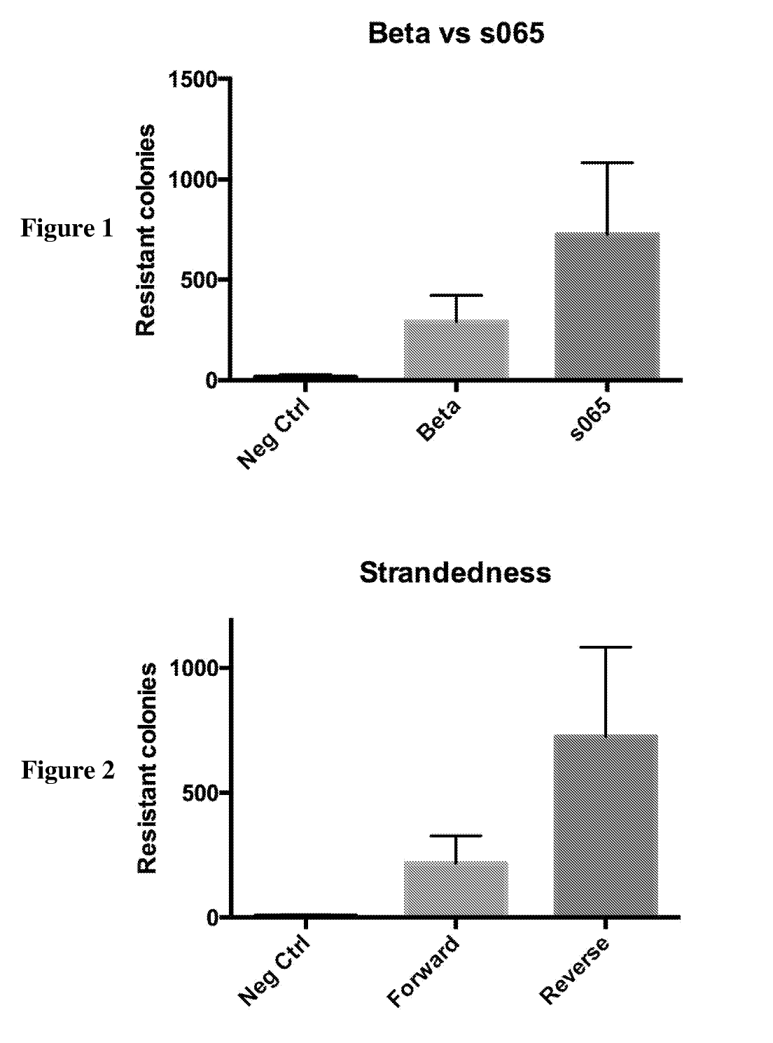

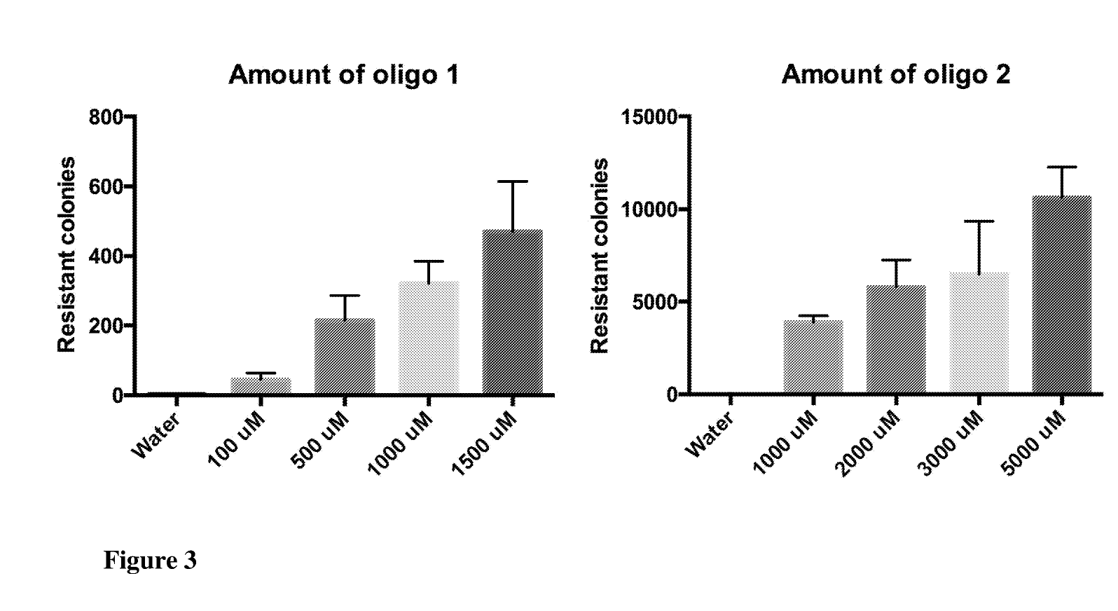

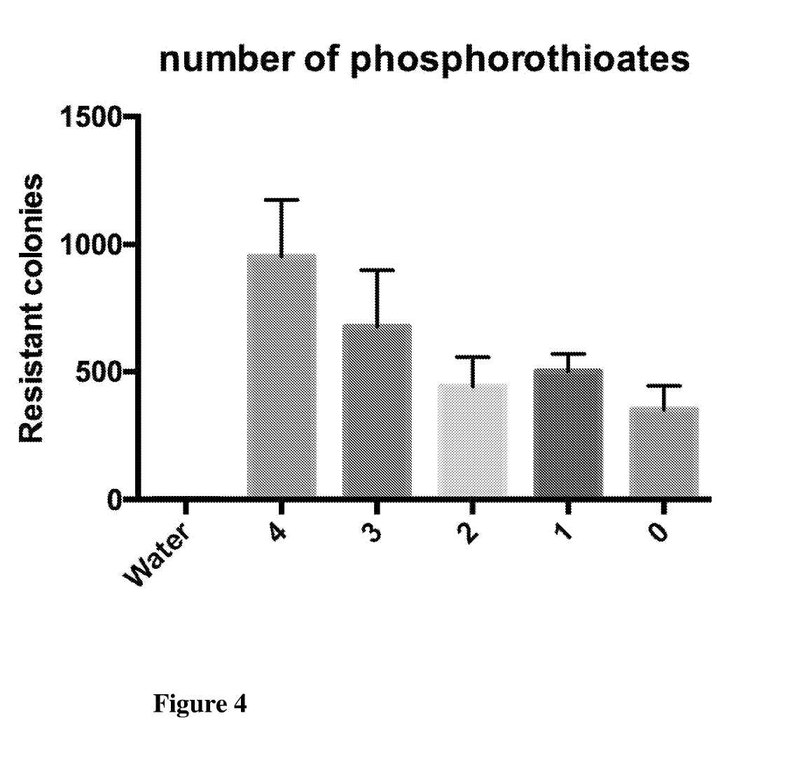

Various recombineering assays were carried out using the protocol as described herein using s065, Vibrio natriegens and single-stranded oligonucleotides and data from the experiments are shown in FIG. 1, FIG. 2, FIG. 3 and FIG. 4 where the y-axis represents the number of colonies yielding a positive recombination event. These assays used a plasmid carrying a spectinomycin resistance marker with a premature stop codon. Catalyzed by s065, the oligonucleotide converts the stop codon back into a functional antibiotic marker. Thus, positive recombination events can be detected by counting number of colonies resistant to spectinomycin. The plasmid sequence is a Genbank file (pRST_brokenspec.gb) shown below:

| | |
|---|---|
| LOCUS | pRST_brokenspec 4761 bp ds-DNA linear 20-JAN-2017 |
| DEFINITION | pRST_brokenspec |
| ACCESSION | |
| KEYWORDS | |
| SOURCE | |
| ORGANISM | other sequences; artificial sequences; vectors. |
| COMMENT | |
| COMMENT | ApEinfo: methylated: 1 |
| FEATURES | Location/Qualifiers |
| misc_feature | 2105 . . . 2496 |
| | /label = oriR6K |
| | /ApEinfo_fwdcolor = #804040 |
| | /ApEinfo_revcolor = #804040 |
| | /ApEinfo_graphicformat = arrow_data {{0 1 2 0 0 −1} { } 0} |
| | width 5 offset 0 |
| misc_feature | 2604 . . . 3419 |
| | /label = Km |
| | /ApEinfo_fwdcolor = #808080 |
| | /ApEinfo_revcolor = #808080 |
| | /ApEinfo_graphicformat = arrow_data {{0 1 2 0 0 −1} { } 0} |
| | width 5 offset 0 |
| misc_feature | 4077 . . . 4077 |
| | /label = stop codon mutation |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| promoter | 3513 . . . 3646 |
| | /created_by = “User” |
| | /label = spectinomycin promoter |
| | /ApEinfo_fwdcolor = #e900ff |
| | /ApEinfo_revcolor = green |
| | /ApEinfo_graphicformat = arrow_data {{0 1 2 0 0 −1} { } 0} |
| | width 5 offset 0 |
| misc_feature | 3647 . . . 4657 |
| | /label = broken spec |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| ORIGIN | |

| | |
|---|---|
| 1 | ggcctcactt gcagcagaac gtgggcagct tgctgaatcg ttctgccaaa gtgagcccgt |
| 61 | aacataatgg cgagtaatac gcattaaggc ggtaactcag ccccgcaggg actagaccta |
| 121 | acgttaggct cagcgctcgc cgctctgatg ctactgcata tccaaagctg ctttagcact |
| 181 | cgcagaagtt cgcttgattg ctcaagcgtt cccgtcagtg aaatgatcct ctttctgata |
| 241 | gcgccagaaa aaactccctt cgtcctgcca agcccatttg gaagtctcag cacacgcaga |
| 301 | gggtaacagc atttgtcatg gatacgttca gcgcccaagg cgcggcgaga gtcgagcaag |
| 361 | cctcttatac tgcgacagcg gcaggtgaga acataagcga cgtagcgtgc ggagtcgcgt |
| 421 | tgttagagcc tgtccgctgt ggtagacccc cgtctagtat tacgggggta aatcccacag |
| 481 | agcctgtgac actcaccttg tattcgcaag cgtagcgcgc cagtgtttga gcgctagcga |
| 541 | gtcttgctaa gcaccatgat ttaagatgct cttggtagaa tgtcttatca gcatactttc |
| 601 | taaaaccatg cttattgctt tttgctcttc ttcatctaac tgttggattt tttttaacct |
| 661 | gagcataagc tcttgatttt catctgtggc ccatcttccg catagttcat caattgagat |
| 721 | ctccagagca tctgcgatct tcacaaggtt ttccattgta ggcaaacctt ccccagattc |
| 781 | gtattttttg tacgatgtta gactaattcc aatttcatca gccatttgtg cctgagtctt |
| 841 | attaattgcc tttctttggt tggctagcct ttcttttatc ttcataacaa tcccctttag |
| 901 | cttgatttat ttctattgta aggctgtttt tttgtacatt agtcttgaaa gtgcgcattg |
| 961 | gttgctgtat tttagctcta aaggttatct tgacaggttt ttgaaggcta atgaaaaagc |
| 1021 | agattttcac tcttgacgaa ttacaactcg atacaaacgc ttctccgttt gtttttgtcg |
| 1081 | attatcttgc ttggtcggtt ccttatgctt cattccgtca cgcgcataag tccgatttgt |
| 1141 | cctcgcttat ctgggcgcct cttcctaagc ctgattaccg tatggctcgc acacctgagc |
| 1201 | aaaaagagaa gttaatcgag ctttataagc agaagtggaa cgttgccatg atggaacgct |
| 1261 | tggaggtctt ttgccttcat gttcttggtc ttcgtatgtc gccttggcgc gataaggggc |
| 1321 | tttatgggta tgaaaactca tgccatttga tgtctaagta ctccaataaa cacgtgggct |
| 1381 | ttgttgcgct agggggaaac cgtaatacct gttacttcca aattgaggga gtagggtgtc |
| 1441 | gaaccgtgtt agagcacacc tctttattcc gtcttcattg gtggctcgat ttattaggtt |
| 1501 | gctctcgtct gtctcgtatt gatttagccg ttgatgactt tcacggtttg tttggccgtg |
| 1561 | agtacgccaa aaaagcctat tccgatgacg cctttcgcac cgctagagcg ggacgtgccc |
| 1621 | ctaacggtgg tgagcgatta gtctctgagc ctaatggcaa aatcatcaat gaatctttcg |
| 1681 | aggtaggctc tcgtgaatct cgcatttact ggcgtatcta caacaaggct gctcagcttg |
| 1741 | gtttagatat gcactggttt cgtaatgagg tcgagcttaa agatatgcct atcgacgttc |
| 1801 | tgctcaatat cgaggggtat tttgcaggtt tgtgcgcgta ctcggcctca attatcaatt |
| 1861 | ccttgcctgt caaggtggtc acaaaaaagc gtcaagtggc gcttgatatc cactcacgca |
| 1921 | ttaagtgggc tcgtcgtcag gtcggtaaga cgttgtttga tatttcaaag cattttggtg |
| 1981 | gtgatttgga aagggtgttt ggggcgttga tttctaagga aattcacgac gattcactca |
| 2041 | accttccaga ttcttatatg aagttaattg atgaaattat gggtgattaa CAGCTGGGCG |
| 2101 | CGCCCCATGT CAGCCGTTAA GTGTTCCTGT GTCACTCAAA |
| | ATTGCTTTGA GAGGCTCTAA |
| 2161 | GGGCTTCTCA GTGCGTTACA TCCCTGGCTT GTTGTCCACA |
| | ACCGTTAAAC CTTAAAGGCT |
| 2221 | TTAAAAGCCT TATATATTCT TTTTTTTCTT ATAAAACTTA |
| | AAACCTTAGA GGCTATTTAA |
| 2281 | GTTGCTGATT TATATTAATT TTATTGTTCA AACATGAGAG |
| | CTTAGTACGT GAAACATGAG |
| 2341 | AGCTTAGTAC GTTAGCCATG AGAGCTTAGT ACGTTAGCCA |
| | TGAGGGTTTA GTTCGTTAAA |
| 2401 | CATGAGAGCT TAGTACGTTA AACATGAGAG CTTAGTACGT |
| | GAAACATGAG AGCTTAGTAC |
| 2461 | GTACTATCAA CAGGTTGAAC TGCTGATCTT CAGATCGACG |
| | TCTTGTGTCT CAAAATCTCT |
| 2521 | GATGTTACAT TGCACAAGAT AAAAATATAT CATCATGAAC |
| | AATAAAACTG TCTGCTTACA |
| 2581 | TAAACAGTAA TACAAGGGGT GTTATGAGCC ATATTCAGCG |
| | TGAAACGAGC TGTAGCCGTC |
| 2641 | CGCGTCTGAA CAGCAACATG GATGCGGATC TGTATGGCTA |
| | TAAATGGGCG CGTGATAACG |
| 2701 | TGGGTCAGAG CGGCGCGACC ATTTATCGTC TGTATGGCAA |
| | ACCGGATGCG CCGGAACTGT |
| 2761 | TTCTGAAACA TGGCAAAGGC AGCGTGGCGA ACGATGTGAC |
| | CGATGAAATG GTGCGTCTGA |
| 2821 | ACTGGCTGAC CGAATTTATG CCGCTGCCGA CCATTAAACA |
| | TTTTATTCGC ACCCCGGATG |
| 2881 | ATGCGTGGCT GCTGACCACC GCGATTCCGG GCAAAACCGC |
| | GTTTCAGGTG CTGGAAGAAT |
| 2941 | ATCCGGATAG CGGCGAAAAC ATTGTGGATG CGCTGGCCGT |
| | GTTTCTGCGT CGTCTGCATA |
| 3001 | GCATTCCGGT GTGCAACTGC CCGTTTAACA GCGATCGTGT |
| | GTTTCGTCTG GCCCAGGCGC |
| 3061 | AGAGCCGTAT GAACAACGGC CTGGTGGATG CGAGCGATTT |
| | TGATGATGAA CGTAACGGCT |
| 3121 | GGCCGGTGGA ACAGGTGTGG AAAGAAATGC ATAAACTGCT |
| | GCCGTTTAGC CCGGATAGCG |
| 3181 | TGGTGACCCA CGGCGATTTT AGCCTGGATA ACCTGATTTT |
| | CGATGAAGGC AAACTGATTG |
| 3241 | GCTGCATTGA TGTGGGCCGT GTGGGCATTG CGGATCGTTA |
| | TCAGGATCTG GCCATTCTGT |
| 3301 | GGAACTGCCT GGGCGAATTT AGCCCGAGCC TGCAAAAACG |
| | TCTGTTTCAG AAATATGGCA |
| 3361 | TTGATAATCC GGATATGAAC AAACTGCAAT TTCATCTGAT |
| | GCTGGATGAA TTTTTCTAAT |
| 3421 | AATTAATTGG GGACCCTAGA GGTCCCCTTT TTTATTTTAA |
| | AAATTTTTTC ACAAAACGGT |
| 3481 | TTACAAGCAT AACTAGTGCG GCCGCAAGCT TGccagccag gacagaaatg |
| | cctcgacttc |
| 3541 | gctgctaccc aaggttgccg ggtgacgcac accgtggaaa cggatgaagg cacgaaccca |
| 3601 | gtggacataa gcctgttcgg ttcgtaagct gtaatgcaag tagcgtatgc gctcacgcaa |
| 3661 | ctggtccaga accttgaccg aacgcagcgg tggtaacggc gcagtggcgg ttttcatggc |
| 3721 | ttgttatgac tgtttttttg gggtacagtc tatgcctcgg gcatccaagc agcaagcgcg |
| 3781 | ttacgccgtg ggtcgatgtt tgatgttatg gagcagcaac gatgttacgc agcagggcag |
| 3841 | tcgccctaaa acaaagttaa acattatgag ggaagcggtg atcgccgaag tatcgactca |
| 3901 | actatcagag gtagttggcg ccatcgagcg ccatctcgaa ccgacgttgc tggccgtaca |
| 3961 | tttgtacggc tccgcagtgg atggcggcct gaagccacac agtgatattg atttgctggt |
| 4021 | tacggtgacc gtaaggcttg atgaaacaac gcggcgagct ttgatcaacg acctttAgga |
| 4081 | aacttcggct tcccctggag agagcgagat tctccgcgct gtagaagtca ccattgttgt |
| 4141 | gcacgacgac atcattccgt ggcgttatcc agctaagcgc gaactgcaat ttggagaatg |
| 4201 | gcagcgcaat gacattcttg caggtatctt cgagccagcc acgatcgaca ttgatctggc |
| 4261 | tatcttgctg acaaaagcaa gagaacatag cgttgccttg gtaggtccag cggcggagga |
| 4321 | actctttgat ccggttcctg aacaggatct atttgaggcg ctaaatgaaa ccttaacgct |
| 4381 | atggaactcg ccgcccgact gggctggcga tgagcgaaat gtagtgctta cgttgtcccg |
| 4441 | catttggtac agcgcagtaa ccggcaaaat cgcgccgaag gatgtcgctg ccggctgggc |
| 4501 | aatggagcgc ctgccggccc agtatcagcc cgtcatactt gaagctagac aggcttatct |
| 4561 | tggacaagaa gaagatcgct tggcctcgcg cgcagatcag ttggaagaat ttgtccacta |
| 4621 | cgtgaaaggc gagatcacca aggtagtcgg caaataaCGG CCTTAATTAA atgatgtttt |
| 4681 | tattccacat ccttagtgcg tattatgtgg cgcgtcatta tgttgagggg cagtcgtcag |
| 4741 | taccattgcg ccagcactga c |

//

FIG. 1 compares λ-Beta versus STX s065 recombinase functionality in V. natriegens. A single-stranded oligonucleotide recombines with a spectinomycin gene, on a plasmid, with a premature stop codon to convert it into a functional spectinomycin gene. As can be seen, s065 performed better than λ-Beta in V. natriegens.

FIG. 2 is directed to oligonucleotide strandedness where recombineering with s065 and oligonucleotides targeting the forward (leading strand, BS_F; cttgatgaaacaacgcggcgagctttgatcaacgacctttTggaaacttcggcttcccctggagagagcgagattctccgcgctgtag aa) or reverse (lagging strand, BS_R; ttctacagcgcggagaatctcgctctctccaggggaagccgaagtttccAaaaggtcgttgatcaaagctcgccgcgttgtttcatcaa g) of DNA replication showed greater targeting of the reverse strand.

FIG. 3 is directed to effect of the amount of oligonucleotide where increasing the amount of oligonucleotide increased s065-mediated recombination in V. natriegens.

FIG. 4 is directed to the effect of the number of phosphorothioates on the oligonucleotide where increasing the number of phosphorothioate bonds enhanced the stability of oligos in vivo. The oligo sequences for the number of phosphorothioates are listed below where an asterisk represents a phosphorothioate bond.

BS_rP3;
t*t*c*tacagcgcggagaatctcgctctctccaggggaagccgaagttt
ccAaaaggtcgttgatcaaagctcgccgcgttgtttcatcaag,
BS_rP2;
t*t*ctacagcgcggagaatctcgctctctccaggggaagccgaagtttc
cAaaaggtcgttgatcaaagctcgccgcgttgtttcatcaag,
BS_rP1;
t*tctacagcgcggagaatctcgctctctccaggggaagccgaagtttcc
Aaaaggtcgttgatcaaagctcgccgcgttgtttcatcaag,
and
BS_rP0;
ttctacagcgcggagaatctcgctctctccaggggaagccgaagtttccA
aaaggtcgttgatcaaagctcgccgcgttgtttcatcaag.

### Example II

### Recombineering of Chromosomal DNA Via Single-Stranded Oligonucleotides

Methods are provided of s065-mediated oligonucleotide recombination on the V. natriegens genome by targeting the chromosomal pyrF-homolog in V. natriegens, herein referred to as pyrF, encoding Orotidine 5′-phosphate decarboxylase. Knocking out pyrF, part of pyrimidine metabolism, leads to resistance to the toxic small molecule 5-FOA. pyrF catalyzes the conversion of 5-fluoroorotic acid (FOA, a uracil analogue) into a highly toxic compound. Intact pyrF confers sensitivity to FOA, and cells lacking functional pyrF are resistant to FOA.

This counterselectable system was established in S. cerevisiae. FOA is used for counterselection in V. natriegens.

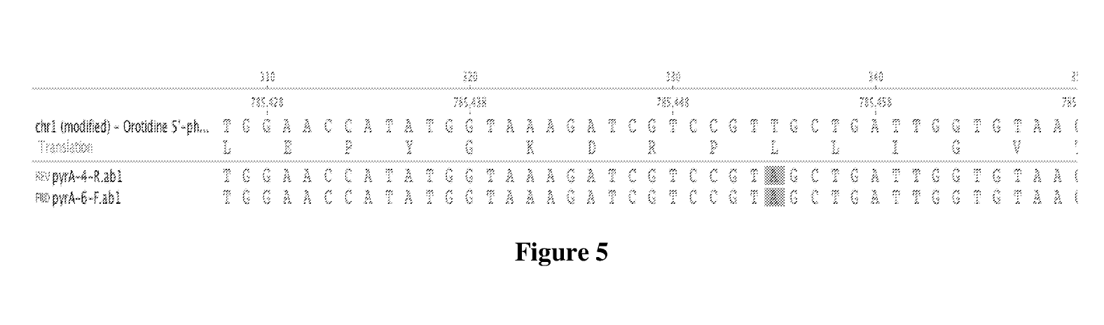

A single-stranded recombineering oligonucleotide was electroporated into a V. natriegens strain expressing s065 to introduce a premature stop codon in the V. natriegens pyrF homolog. pyrF mutants carrying the oligonucleotide sequence were isolated on solid media plates containing lmg/ml 5-FOA. V. natriegens mutants were generated carrying a functional knock-out pyrF allele, which can be used as a non-antibiotic counter selectable marker in cloning and recombineering strains. FIG. 5 is directed to recombineering on a chromosome and depicts results of Sanger sequencing of V. natriegens pyrF mutant colonies isolated by 5-FOA selection following ssDNA oligonucleotide recombination. The oligo sequences are Vnat_pyrF_2; cagcatctcgtgagattctggaaccatatggtaaagatcgtccgtAgctgattggtgtaacggtactaaccagcatggaacagagtgat t, and Vnat_pyrR_2; aatcactctgttccatgctggttagtaccgttacaccaatcagcTacggacgatctttaccatatggttccagaatctcacgagatgctg.

### Example III

### Recombination of Chromosomal DNA Via Double-Stranded DNA Cassette

Recombineering of V. natriegens via double-stranded DNA cassettes was carried out using expression of three genes: two genes from SXT and one from λ-phage: s065 recombinase (Beta homolog), s066 exonuclease (exo homolog) and gam, respectively. See Court, D. L., Sawitzke, J. A. & Thomason, L. C., 2002. Genetic Engineering Using Homologous Recombination 1. Annual review of genetics, 36(1), pp. 361-388 hereby incorporated by reference in its entirety. The expression of s065, s066, and gam is sufficient for double-stranded recombineering in V. natriegens.

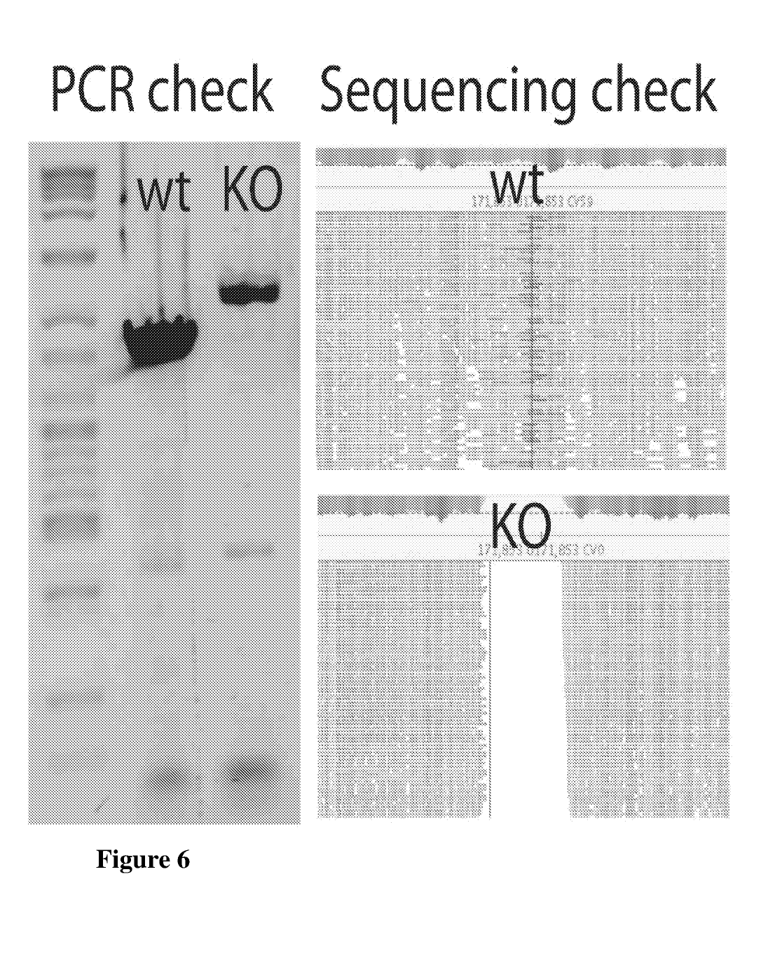

One of the two extracellular DNAse genes, dns, was deleted on the V. natriegens genome. This gene is homologous to endA in E. coli. Strains of E. coli with the endAl allele are functionally deficient in DNAse activity and have found broad utility as cloning and sequencing strains. See Taylor, R. G., Walker, D. C. & Mclnnes, R. R., 1993. E. coli host strains significantly affect the quality of small scale plasmid DNA preparations used for sequencing. Nucleic acids research, 21(7), pp. 1677-1678 hereby incorporated by reference in its entirety. A V. natriegens dns deletion mutant has improved plasmid yield and stability. See Weinstock, M. T. et al., 2016. Vibrio natriegens as a fast-growing host for molecular biology. Nature methods, 13(10), pp. 849-851. To demonstrate precise deletion of chromosomal DNA, a double-stranded DNA cassette was constructed which consisted of the spectinomycin antibiotic gene flanked by 500 bp on both ends immediately upstream and downstream of the dns gene. To increase the in vivo stability of the double-stranded DNA cassette, such as protection from exonucleases, phosphorothioates were added to proximal 5′ end of one or both strands. 1 ug of this double-stranded DNA cassette was electrotransformed into a V. natriegens strain expressing s065, s66, and gam and colonies resistant to spectinomycin were screened for successful recombination between the double-stranded DNA cassette and the chromosomal DNA. Recombination of the double-stranded DNA cassette into the genome was verified by PCR and next-generation whole-genome sequencing. FIG. 6 is directed to gene deletion by insertion of antibiotic marker into the V. natriegens genome by SXT-mediated recombination. PCR check (left panel) validated insertion of dsDNA cassette at the dns gene locus, resulting in deletion of dns and insertion of spectinomycin resistance marker. The wildtype (left) shows a 1.7 kb band whereas the KO mutant (right) shows a 2.1 kb band. Sequencing check (right panel) was performed by next-generation Illumina sequencing of wildtype and dns mutant V. natriegens cells. Sequencing reads map to the dns locus for wildtype (top) but no reads matching the dns gene can be found for the KO mutant (bottom), confirming complete deletion of the dns gene.

The sequence of the dns cassette is a Genbank file (dnsCassette_500 bp_homology.gb):

| | |
|---|---|
| LOCUS | dnsCassette_500b 2145 bp ds-DNA linear 20-JAN-2017 |
| DEFINITION. | |
| ACCESSION | |
| VERSION | |
| SOURCE. | |
| ORGANISM. | |
| COMMENT | |
| COMMENT | |
| COMMENT | ApEinfo: methylated: 1 |
| FEATURES | Location/Qualifiers |
| promoter | 501 . . . 634 |
| | /created_by = “User” |
| | /label = spectinomycin promoter |
| | /ApEinfo_fwdcolor = #e900ff |
| | /ApEinfo_revcolor = green |
| | /ApEinfo_graphicformat = arrow_data {{0 1 2 0 0 −1} { } 0} |
| | width 5 offset 0 |
| gene | 635 . . . 1645 |
| | /created_by = “User” |
| | /modified_by = “User” |
| | /label = Spectinomycin |
| | /ApEinfo_fwdcolor = pink |
| | /ApEinfo_revcolor = pink |
| | /ApEinfo_graphicformat = arrow_data {{0 1 2 0 0 −1} { } 0} |
| | width 5 offset 0 |
| misc_feature | 1 . . . 500 |
| | /label = Vnat_genome_upstream_dns |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| misc_feature | 1646 . . . 2145 |
| | /label = Vnat_genome_downstream_dns |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| ORIGIN | |

| | |
|---|---|
| 1 | aaagcgtacc ttcagctcaa tgagattcgc cttaacccgg ttttatttaa agaaaacacc |
| 61 | caagcgttct tgcaagaagt gataccgcat gaggtcgctc acttaatcac atatcaggtt |
| 121 | tacggtcgcg tccgtcctca tggcaaagag tggcaaaccg taatggaatc cgtatttaac |
| 181 | gttccggcca aaaccacaca tagtttcgaa gtctcttccg ttcaaggcaa aaccttcgaa |
| 241 | taccgatgtc gctgcacgac atatcccctt tctattcgcc gtcacaacaa agtgctgcgc |
| 301 | aaacaagccg tgtattcgtg tcaaaaatgt cgtcagcctc ttagcttcac tggtgtccag |
| 361 | ctttcctaat cctcagttca attaagtctc aataggaaat attgaccaac atttcttttg |
| 421 | ttattattaa cttgcttatt acgaaagcta atatctgagt gatagaatgg ataaagtcat |
| 481 | actttttaaa gactttaact ccagccagga cagaaatgcc tcgacttcgc tgctacccaa |
| 541 | ggttgccggg tgacgcacac cgtggaaacg gatgaaggca cgaacccagt ggacataagc |
| 601 | ctgttcggtt cgtaagctgt aatgcaagta gcgtatgcgc tcacgcaact ggtccagaac |
| 661 | cttgaccgaa cgcagcggtg gtaacggcgc agtggcggtt ttcatggctt gttatgactg |
| 721 | tttttttggg gtacagtcta tgcctcgggc atccaagcag caagcgcgtt acgccgtggg |
| 781 | tcgatgtttg atgttatgga gcagcaacga tgttacgcag cagggcagtc gccctaaaac |
| 841 | aaagttaaac attatgaggg aagcggtgat cgccgaagta tcgactcaac tatcagaggt |
| 901 | agttggcgcc atcgagcgcc atctcgaacc gacgttgctg gccgtacatt tgtacggctc |
| 961 | cgcagtggat ggcggcctga agccacacag tgatattgat ttgctggtta cggtgaccgt |
| 1021 | aaggcttgat gaaacaacgc ggcgagcttt gatcaacgac cttttggaaa cttcggcttc |
| 1081 | ccctggagag agcgagattc tccgcgctgt agaagtcacc attgttgtgc acgacgacat |
| 1141 | cattccgtgg cgttatccag ctaagcgcga actgcaattt ggagaatggc agcgcaatga |
| 1201 | cattcttgca ggtatcttcg agccagccac gatcgacatt gatctggcta tcttgctgac |
| 1261 | aaaagcaaga gaacatagcg ttgccttggt aggtccagcg gcggaggaac tctttgatcc |
| 1321 | ggttcctgaa caggatctat ttgaggcgct aaatgaaacc ttaacgctat ggaactcgcc |
| 1381 | gcccgactgg gctggcgatg agcgaaatgt agtgcttacg ttgtcccgca tttggtacag |
| 1441 | cgcagtaacc ggcaaaatcg cgccgaagga tgtcgctgcc ggctgggcaa tggagcgcct |
| 1501 | gccggcccag tatcagcccg tcatacttga agctagacag gcttatcttg gacaagaaga |
| 1561 | agatcgcttg gcctcgcgcg cagatcagtt ggaagaattt gtccactacg tgaaaggcga |
| 1621 | gatcaccaag gtagtcggca aataatcctc accaatcgcg acaatcgcta atctttctgt |
| 1681 | ttgaggcgtt tcatttactc caattgaaac gcctcttgcc ccttgttttt tcgatggaaa |
| 1741 | gcatccatgt taggaactaa gtttattctc ttgctggaaa tctcatgcgt atccctcgaa |
| 1801 | tttatcatcc agaaaccatt caccaacttg gtacactcgc tttaagtgac gacgccgctg |
| 1861 | gccatattgg ccgcgtactt cgtatgaagg aaggtcagga agttctccta tttgacggta |
| 1921 | gtggtgcaga gtttcccgca gttatcgcag aagtcagcaa aaagaatgtc ctcgtagaca |
| 1981 | tctctgagcg cgtagagaac agcattgaat cccctttgga tcttcaccta ggacaggtga |
| 2041 | tttcacgagg cgacaagatg gagttcacca ttcagaagtc agtcgaactc ggagtaaata |
| 2101 | ccatcactcc ccttatttct gaacgttgtg gcgtaaagct cgatc |

//

### Example IV

### Sequences

s065 is deposited in UniProt as Q8KQW0. The amino acid sequence follows:

>tr|Q8KQW0|Q8KQW0_VIBCL Putative DNA recombination
protein OS = Vibrio cholerae
GN = s065 PE = 4 SV = 1
MEKPKLIQRFAERFSVDPNKLFDTLKATAFKQRDGSAPTNEQMMALLVV
ADQYGLNPFTK
EIFAFPDKQAGIIPVVGVDGWSRIINQHDQFDGMEFKTSENKVSLDGAK
ECPEWMECIIY
RRDRSHPVKITEYLDEVYRPPFEGNGKNGPYRVDGPWQTHTKRMLRHKS
MIQCSRIAFGF
VGIFDQDEAERIIEGQATHIVEPSVIPPEQVDDRTRGLVYKLIERAEAS
NAWNSALEYAN
EHFQGVELTFAKQEIFNAQQQAAKALTQPLAS

s066 is deposited in UniProt as Q8KQV9. The amino acid sequence follows:

>tr|Q8KQV9|Q8KQV9_VIBCL Endonuclease
OS = Vibrio cholerae GN = s066 PE = 4 SV = 1
MKVIDLSQRTPAWHQWRIAGVTASEAPIIMGRSPYKTPWRLWAEKTGFVL
PEDLSNNPNV
LRGIRLEPQARRAFENAHNDFLLPLCAEADHNAIFRASFDGINDAGEPVE
LKCPCQSVFE
DVQAHREQSEAYQLYWVQVQHQILVANSTRGWLVFYFEDQLIEFEIQRDA
AFLTELQETA
LQFWELVQTKKEPSKCPEQDCFVPKGEAQYRWTSLSRQYCSAHAEVVRLE
NHIKSLKEEM
RDAQSKLVAMMGNYAHADYAGVKLSRYMMAGTVDYKQLATDKLGELDEQV
LAAYRKAPQE
RLRISTNKPEQPVETPIKISLEQENLVLPGDSPSSFYF

The following s065 and s066 cassette was synthesized (s065 in Italics, s066 in Bold) and inserted downstream of an inducible promoter (IPTG/arabinose/heat/etc) and gam on an RSF1010 origin plasmid:

atgaaaaaccaagtaacactcataggctatgttggctctgagccagaga
cgcgagcctatccatcaggtgatttagtgaccagcatttcactggccac
ttctgagaaatggcgcgaccgtcaatccaatgagctcaaagagcatacg
gaatggcatcgggtcgtttttcgagatcgtggtggattaaagttagggc
tcagggcaaaagatttaatccaaaaaggagcgaagctttttgttcaagg
gcctcagcgcacgcgctcatgggagaaagatggcattaagcatcgattg
accgaagtggacgcggacgagtttctgcttcttgataatgtgaacaaag
catctgagccatcagcggcggatgatgcaggctcccaaactaattgggc
acaaacttatcctgaaccagatttttaaccgagcaaaaacgctttaacc
cagccgggagtactttcccgtcaggggcagactcccactttgattgtcg
gagtccacaatggaaaaaccaaagctaatccaacgctttgctgagcgct
ttagtgtcgatccaaacaaactgttcgataccctaaaagcaacagcatt
taagcaacgtgacggtagtgcaccgaccaatgagcagatgatggcgctc
ttggtggttgcagatcagtacggcttgaaccctttcaccaaagagattt
ttgcgttccctgataagcaagctggaattattccagtggtaggtgtcga
tggatggtctcgcatcatcaatcaacacgaccagtttgatggcatggag
tttaagacttcagaaaacaaagtctccctggatggcgcgaaagaatgcc
cggaatggatggaatgcattatctaccggcgcgaccgttcgcacccagt
caaaatcactgaatacctggatgaagtctatcgaccgccttttgagggt
aacggaaaaaatggcccttaccgtgtagatggtccatggcagacgcaca
ctaagcgaatgctaagacataaatccatgatccagtgttcccgcattgc
gtttggctttgtgggaattttcgatcaagacgaagcggagcgaattatc
gaaggccaagcaacacacattgttgagccatcggtgattccacccgagc
aagttgatgatcgaacccgagggcttgtttacaagcttatcgagcgggc
ggaagcttcaaacgcatggaatagtgcattggaatacgccaatgaacat
tttcaaggtgttgaactgacgtttgcgaaacaagaaatatttaatgcac
agcaacaagcagccaaagcgctcacacagcctttagcttcttag

See GenBank file (“pRSF_lac_gam_s065_s066.gb”) for full plasmid with annotations.

s064 is deposited in uniprot as A0A0X1L3H7.

Amino acid sequence:
MKNQVTLIGYVGSEPETRAYPSGDLVTSISLATSEKWRDRQSNELKEHT
EWHRVVFRDRGGLKLGLRAKDLIQKGAKLFVQGPQRTRSWEKDGIKHRL
TEVDADEFLLLDNVNKASEPSAADDAGSQTNWAQTYPEPDF
DNA Sequence:
ATGAAAAACCAAGTAACACTCATAGGCTATGTTGGCTCTGAGCCAGAGA
CGCGAGCCTATCCATCAGGTGATTTAGTGACCAGCATTTCACTGGCCAC
TTCTGAGAAATGGCGCGACCGTCAATCCAATGAGCTCAAAGAGCATACG
GAATGGCATCGGGTCGTTTTTCGAGATCGTGGTGGATTAAAGTTAGGGC
TCAGGGCAAAAGATTTAATCCAAAAAGGAGCGAAGCTTTTTGTTCAAGG
GCCTCAGCGCACGCGCTCATGGGAGAAAGATGGCATTAAGCATCGATTG
ACCGAAGTGGACGCGGACGAGTTTCTGCTTCTTGATAATGTGAACAAAG
CATCTGAGCCATCAGCGGCGGATGATGCAGGCTCCCAAACTAATTGGGC
ACAAACTTATCCTGAACCAGATTTTTAA

### Example V

### Representative Plasmids Including all Recombineering Proteins and SSB Protein

| | |
|---|---|
| LOCUS | pRSF_lac_gam_s06 10892 bp ds-DNA circular 05-OCT-2016 |
| DEFINITION | Reconnbineering helper plasmid for Vibrio natriegens, complete |
| | sequence. |
| SOURCE | Derived from Red-reconnbineering helper plasmid RSFRedkan |
| ORGANISM | Recombineering helper plasmid for Vibrio natriegens, complete |
| | sequence; artificial sequences; vectors. |
| REFERENCE | 1 (bases 1 to 11037) |
| AUTHORS | Lee, HH., Ostrov, N., Church, GM. |
| TITLE | Unpublished |
| REFERENCE | 2 (bases 1 to 11037) |
| COMMENT | |
| COMMENT | ApEinfo:nnethylated:1 |
| FEATURES | Location/Qualifiers |
| CDS | 1087 . . . 1905 |
| | /label = s066 |
| | /ApEinfo_fwdcolor = cyan |
| | /ApEinfo_revcolor = green |
| | /ApEinfo_graphicfornnat = arrow_data {{0 1 2 0 0 −1} { } 0} |
| | width 5 offset 0 |
| promoter | 13 . . . 138 |
| | /note = “PlacUV5” |
| | /label = PlacUV5 |
| | /ApEinfo_fwdcolor = pink |
| | /ApEinfo_revcolor = pink |
| | /ApEinfo_graphicformat = arrow_data {{0 1 2 0 0 −1} { } 0} |
| | width 5 offset 0 |
| CDS | 588 . . . 1007 |
| | /label = s065 |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| gene | 166 . . . 582 |
| | /gene = “gam” |
| | /label = gam |
| | /ApEinfo_fwdcolor = pink |
| | /ApEinfo_revcolor = pink |
| | /ApEinfo_graphicformat = arrow_data {{0 1 2 0 0 −1} { } 0} |
| | width 5 offset 0 |
| CDS | 166 . . . 582 |
| | /gene = “gam” |
| | /note = “derived fronn Escherichia coli lambda phage” |
| | /codon_start = 1 |
| | /transl_table = 11 |
| | /product = “Gam” |
| | /protein_id = “ACJ06683.1” |
| | /db_xref = “GI: 210076662” |
| | /translation = “MDINTETEIKQKHSLTPFPVFLISPAFRGRYFHSYFRSSAMNAY |
| | YIQDRLEAQSWARHYQQLAREEKEAELADDMEKGLPQHLFESLCIDHLQRHGASKKSI |
| | TRAFDDDVEFQERMAEHIRYMVETIAHHQVDIDSEV” |
| | /label = gam(1) |
| | /ApEinfo_label = gam |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| terminator | 1906 . . . 2164 |
| | /note = “tL3 terminator of Escherichia coli lambda phage” |
| | /label = tL3 terminator of Escherichia coli lambda phage |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| promoter | 8275 . . . 8400 |
| | /note = “PlacUV5” |
| | /label = PlacUV5(1) |
| | /ApEinfo_label = PlacUV5 |
| | /ApEinfo_fwdcolor = pink |
| | /ApEinfo_revcolor = pink |
| | /ApEinfo_graphicformat = arrow_data {{0 1 2 0 0 −1} { } 0} |
| | width 5 offset 0 |
| gene | 8445 . . . 9527 |
| | /gene = “lacl” |
| | /label = lacl |
| | /ApEinfo_fwdcolor = pink |
| | /ApEinfo_revcolor = pink |
| | /ApEinfo_graphicfornnat = arrow_data {{0 1 2 0 0 −1} { } 0} |
| | width 5 offset 0 |
| CDS | 8445 . . . 9527 |
| | /gene = “lacl” |
| | /note = “repressor of Escherichia coli lactose operon” |
| | /codon_start = 1 |
| | /transl_table = 11 |
| | /product = “Lacl” |
| | /protein_id = “ACJ06686.1” |
| | /db_xref = “GI: 210076665” |
| | /translation = “MKPVTLYDVAEYAGVSYQTVSRVVNQASHVSAKTREKVEAAMAE |
| | LNYIPNRVAQQLAGKQSLLIGVATSSLALHAPSQIVAAIKSRADQLGASVVVSMVERS |
| | GVEACKAAVHNLLAQRVSGLIINYPLDDQDAIAVEAACTNVPALFLDVSDQTPINSII |
| | FSHEDGTRLGVEHLVALGHQQIALLAGPLSSVSARLRLAGWHKYLTRNQIQPIAEREG |
| | DWSAMSGFQQTMQMLNEGIVPTAMLVANDQMALGAMRAITESGLRVGADISVVGYDDT |
| | EDSSCYIPPLTTIKQDFRLLGQTSVDRLLQLSQGQAVKGNQLLPVSLVKRKTTLAPNT |
| | QTASPRALADSLMQLARQVSRLESGQ” |
| | /label = Lacl |
| | /ApEinfo_fwdcolor = cyan |
| | /ApEinfo_reycolor = green |
| | /ApEinfo_graphicformat = arrow_data {{0 1 2 0 0 −1} { } 0} |
| | width 5 offset 0 |
| CDS | 9651 . . . 10466 |
| | /function = “resistance to kanamycin” |
| | /note = “kanannycin kinase” |
| | /codon_start = 1 |
| | /transl_table = 11 |
| | /product = “aminoglycoside phosphotransferase” |
| | /protein_id = “ACJ06687.1” |
| | /db_xref = “GI: 210076666” |
| | /translation = “MSHIQRETSCSRPRLNSNMDADLYGYKWARDNVGQSGATIYRLY |
| | GKPDAPELFLKHGKGSVANDVTDEMVRLNWLTEFM PLPTIKHFIRTPDDAWLLTTAIP |
| | GKTAFQVLEEYPDSGENIVDALAVFLRRLHSIPVCNCPFNSDRVFRLAQAQSRMNNGL |
| | VDASDFDDERNGWPVEQVWKEMHKLLPFSPDSVVTHGDFSLDNLIFDEGKLIGCIDVG |
| | RVGIADRYQDLAILWNCLGEFSPSLQKRLFQKYGIDNPDM NKLQFHLMLDEFF” |
| | /label = aminoglycoside phosphotransferase |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| misc_feature | 10488 . . . 10505 |
| | /note = “recognition site of I-Scel restrictase” |
| | /label = recognition site of I-Scel restrictase |
| | /ApEinfo_fwdcolor = #0039ff |
| | /ApEinfo_revcolor = #0004ff |
| | /ApEinfo_graphicformat = arrow_data {{0 1 2 0 0 −1} { } 0} |
| | width 5 offset 0 |
| terminator | 10576 . . . 10857 |
| | /note = “derived from Escherichia coli rrnB operon” |
| | /label = derived from Escherichia coli rrnB operon |
| | /ApEinfo_fwdcolor = cyan |
| | /ApEinfo_revcolor = green |
| | /ApEinfo_graphicformat = arrow_data {{0 1 2 0 0 −1} { } 0} |
| | width 5 offset 0 |
| ORIGIN | |

| 1 | GGTACCAGAT CTGCGGGCAG TGAGCGCAAC GCAATTAATG TGAGTTAGCT CACTCATTAG |
|---|---|
| 61 | GCACCCCAGG CTTTACACTT TATGCTTCCG GCTCGTATAA TGTGTGGAAT TGTGAGCGGA |
| 121 | TAACAATTTC ACACAGGAGG ATCCCGATCG AGGAGGTTAT AAAAAATGGA TATTAATACT |
| 181 | GAAACTGAGA TCAAGCAAAA GCATTCACTA ACCCCCTTTC CTGTTTTCCT AATCAGCCCG |
| 241 | GCATTTCGCG GGCGATATTT TCACAGCTAT TTCAGGAGTT CAGCCATGAA CGCTTATTAC |
| 301 | ATTCAGGATC GTCTTGAGGC TCAGAGCTGG GCGCGTCACT ACCAGCAGCT CGCCCGTGAA |
| 361 | GAGAAAGAGG CAGAACTGGC AGACGACATG GAAAAAGGCC TGCCCCAGCA CCTGTTTGAA |
| 421 | TCGCTATGCA TCGATCATTT GCAACGCCAC GGGGCCAGCA AAAAATCCAT TACCCGTGCG |
| 481 | TTTGATGACG ATGTTGAGTT TCAGGAGCGC ATGGCAGAAC ACATCCGGTA CATGGTTGAA |
| 541 | ACCATTGCTC ACCACCAGGT TGATATTGAT TCAGAGGTAT AAAACGAatg aaaaaccaag |
| 601 | taacactcat aggctatgtt ggctctgagc cagagacgcg agcctatcca tcaggtgatt |
| 661 | tagtgaccag catttcactg gccacttctg agaaatggcg cgaccgtcaa tccaatgagc |
| 721 | tcaaagagca tacggaatgg catcgggtcg tttttcgaga tcgtggtgga ttaaagttag |
| 781 | ggctcagggc aaaagattta atccaaaaag gagcgaagct ttttgttcaa gggcctcagc |
| 841 | gcacgcgctc atgggagaaa gatggcatta agcatcgatt gaccgaagtg gacgcggacg |
| 901 | agtttctgct tcttgataat gtgaacaaag catctgagcc atcagcggcg gatgatgcag |
| 961 | gctcccaaac taattgggca caaacttatc ctgaaccaga tttttaaccg agcaaaaacg |
| 1021 | ctttaaccca gccgggagta ctttcccgtc aggggcagac tcccactttg attgtcggag |
| 1081 | tccacaatgg aaaaaccaaa gctaatccaa cgctttgctg agcgctttag tgtcgatcca |
| 1141 | aacaaactgt tcgataccct aaaagcaaca gcatttaagc aacgtgaTgg tagtgcaccg |
| 1201 | accaatgagc agatgatggc gctcttggtg gttgcagatc agtacggctt gaaccctttc |
| 1261 | accaaagaga tttttgcgtt ccctgataag caagctggaa ttattccagt ggtaggtgtc |
| 1321 | gatggatggt ctcgcatcat caatcaacac gaccagtttg atggcatgga gtttaagact |
| 1381 | tcagaaaaca aagtctccct ggatggcgcg aaagaatgcc cggaatggat ggaatgcatt |
| 1441 | atctaccggc gcgaccgttc gcacccagtc aaaatcactg aatacctgga tgaagtctat |
| 1501 | cgaccgcctt ttgagggtaa cggaaaaaat ggcccttacc gtgtagatgg tccatggcag |
| 1561 | acgcacacta agcgaatgct aagacataaa tccatgatcc agtgttcccg cattgcgttt |
| 1621 | ggctttgtgg gaattttcga tcaagacgaa gcggagcgaa ttatcgaagg ccaagcaaca |
| 1681 | cacattgttg agccatcggt gattccaccc gagcaagttg atgatcgaac ccgagggctt |
| 1741 | gtttacaagc ttatcgagcg ggcggaagct tcaaacgcat ggaatagtgc attggaatac |
| 1801 | gccaatgaac attttcaagg tgttgaactg acgtttgcga aacaagaaat atttaatgca |
| 1861 | cagcaacaag cagccaaagc gctcacacag cctttagctt cttagCGCAT CCTCACGATA |
| 1921 | ATATCCGGGT AGGCGCAATC ACTTTCGTCT ACTCCGTTAC AAAGCGAGGC TGGGTATTTC |
| 1981 | CCGGCCTTTC TGTTATCCGA AATCCACTGA AAGCACAGCG GCTGGCTGAG GAGATAAATA |
| 2041 | ATAAACGAGG GGCTGTATGC ACAAAGCATC TTCTGTTGAG TTAAGAACGA GTATCGAGAT |
| 2101 | GGCACATAGC CTTGCTCAAA TTGGAATCAG GTTTGTGCCA ATACCAGTAG AAACAGACGA |
| 2161 | AGAAGCGGCC GCGATCAAGC AGGTGCGACA GACGTCATAC TAGATATCAA GCGACTTCTC |
| 2221 | CTATCCCCTG GGAACACATC AATCTCACCG GAGAATATCG CTGGCCAAAG CCTTAGCGTA |
| 2281 | GGATTCCGCC CCTTCCCGCA AACGACCCCA AACAGGAAAC GCAGCTGAAA CGGGAAGCTC |
| 2341 | AACACCCACT GACGCATGGG TTGTTCAGGC AGTACTTCAT CAACCAGCAA GGCGGCACTT |
| 2401 | TCGGCCATCC GCCGCGCCCC ACAGCTCGGG CAGAAACCGC GACGCTTACA GCTGAAAGCG |
| 2461 | ACCAGGTGCT CGGCGTGGCA AGACTCGCAG CGAACCCGTA GAAAGCCATG CTCCAGCCGC |
| 2521 | CCGCATTGGA GAAATTCTTC AAATTCCCGT TGCACATAGC CCGGCAATTC CTTTCCCTGC |
| 2581 | TCTGCCATAA GCGCAGCGAA TGCCGGGTAA TACTCGTCAA CGATCTGATA GAGAAGGGTT |
| 2641 | TGCTCGGGTC GGTGGCTCTG GTAACGACCA GTATCCCGAT CCCGGCTGGC CGTCCTGGCC |
| 2701 | GCCACATGAG GCATGTTCCG CGTCCTTGCA ATACTGTGTT TACATACAGT CTATCGCTTA |
| 2761 | GCGGAAAGTT CTTTTACCCT CAGCCGAAAT GCCTGCCGTT GCTAGACATT GCCAGCCAGT |
| 2821 | GCCCGTCACT CCCGTACTAA CTGTCACGAA CCCCTGCAAT AACTGTCACG CCCCCCTGCA |
| 2881 | ATAACTGTCA CGAACCCCTG CAATAACTGT CACGCCCCCA AACCTGCAAA CCCAGCAGGG |
| 2941 | GCGGGGGCTG GCGGGGTGTT GGAAAAATCC ATCCATGATT ATCTAAGAAT AATCCACTAG |
| 3001 | GCGCGGTTAT CAGCGCCCTT GTGGGGCGCT GCTGCCCTTG CCCAATATGC CCGGCCAGAG |
| 3061 | GCCGGATAGC TGGTCTATTC GCTGCGCTAG GCTACACACC GCCCCACCGC TGCGCGGCAG |
| 3121 | GGGGAAAGGC GGGCAAAGCC CGCTAAACCC CACACCAAAC CCCGCAGAAA TACGCTGGAG |
| 3181 | CGCTTTTAGC CGCTTTAGCG GCCTTTCCCC CTACCCGAAG GGTGGGGGCG CGTGTGCAGC |
| 3241 | CCCGCAGGGC CTGTCTCGGT CGATCATTCA GCCCGGCTCA TCCTTCTGGC GTGGCGGCAG |
| 3301 | ACCGAACAAG GCGCGGTCGT GGTCGCGTTC AAGGTACGCA TCCATTGCCG CCATGAGCCG |
| 3361 | ATCCTCCGGC CACTCGCTGC TGTTCACCTT GGCCAAAATC ATGGCCCCCA CCAGCACCTT |
| 3421 | GCGCCTTGTT TCGTTCTTGC GCTCTTGCTG CTGTTCCCTT GCCCGCACCC GCTGAATTTC |
| 3481 | GGCATTGATT CGCGCTCGTT GTTCTTCGAG CTTGGCCAGC CGATCCGCCG CCTTGTTGCT |
| 3541 | CCCCTTAACC ATCTTGACAC CCCATTGTTA ATGTGCTGTC TCGTAGGCTA TCATGGAGGC |
| 3601 | ACAGCGGCGG CAATCCCGAC CCTACTTTGT AGGGGAGGGC GCACTTACCG GTTTCTCTTC |
| 3661 | GAGAAACTGG CCTAACGGCC ACCCTTCGGG CGGTGCGCTC TCCGAGGGCC ATTGCATGGA |
| 3721 | GCCGAAAAGC AAAAGCAACA GCGAGGCAGC ATGGCGATTT ATCACCTTAC GGCGAAAACC |
| 3781 | GGCAGCAGGT CGGGCGGCCA ATCGGCCAGG GCCAAGGCCG ACTACATCCA GCGCGAAGGC |
| 3841 | AAGTATGCCC GCGACATGGA TGAAGTCTTG CACGCCGAAT CCGGGCACAT GCCGGAGTTC |
| 3901 | GTCGAGCGGC CCGCCGACTA CTGGGATGCT GCCGACCTGT ATGAACGCGC CAATGGGCGG |
| 3961 | CTGTTCAAGG AGGTCGAATT TGCCCTGCCG GTCGAGCTGA CCCTCGACCA GCAGAAGGCG |
| 4021 | CTGGCGTCCG AGTTCGCCCA GCACCTGACC GGTGCCGAGC GCCTGCCGTA TACGCTGGCC |
| 4081 | ATCCATGCCG GTGGCGGCGA GAACCCGCAC TGCCACCTGA TGATCTCCGA GCGGATCAAT |
| 4141 | GACGGCATCG AGCGGCCCGC CGCTCAGTGG TTCAAGCGGT ACAACGGCAA GACCCCGGAG |
| 4201 | AAGGGCGGGG CACAGAAGAC CGAAGCGCTC AAGCCCAAGG CATGGCTTGA GCAGACCCGC |
| 4261 | GAGGCATGGG CCGACCATGC CAACCGGGCA TTAGAGCGGG CTGGCCACGA CGCCCGCATT |
| 4321 | GACCACAGAA CACTTGAGGC GCAGGGCATC GAGCGCCTGC CCGGTGTTCA CCTGGGGCCG |
| 4381 | AACGTGGTGG AGATGGAAGG CCGGGGCATC CGCACCGACC GGGCAGACGT GGCCCTGAAC |
| 4441 | ATCGACACCG CCAACGCCCA GATCATCGAC TTACAGGAAT ACCGGGAGGC AATAGACCAT |
| 4501 | GAACGCAATC GACAGAGTGA AGAAATCCAG AGGCATCAAC GAGTTAGCGG AGCAGATCGA |
| 4561 | ACCGCTGGCC CAGAGCATGG CGACACTGGC CGACGAAGCC CGGCAGGTCA TGAGCCAGAC |
| 4621 | CCAGCAGGCC AGCGAGGCGC AGGCGGCGGA GTGGCTGAAA GCCCAGCGCC AGACAGGGGC |
| 4681 | GGCATGGGTG GAGCTGGCCA AAGAGTTGCG GGAGGTAGCC GCCGAGGTGA GCAGCGCCGC |
| 4741 | GCAGAGCGCC CGGAGCGCGT CGCGGGGGTG GCACTGGAAG CTATGGCTAA CCGTGATGCT |
| 4801 | GGCTTCCATG ATGCCTACGG TGGTGCTGCT GATCGCATCG TTGCTCTTGC TCGACCTGAC |
| 4861 | GCCACTGACA ACCGAGGACG GCTCGATCTG GCTGCGCTTG GTGGCCCGAT GAAGAACGAC |
| 4921 | AGGACTTTGC AGGCCATAGG CCGACAGCTC AAGGCCATGG GCTGTGAGCG CTTCGATATC |
| 4981 | GGCGTCAGGG ACGCCACCAC CGGCCAGATG ATGAACCGGG AATGGTCAGC CGCCGAAGTG |
| 5041 | CTCCAGAACA CGCCATGGCT CAAGCGGATG AATGCCCAGG GCAATGACGT GTATATCAGG |
| 5101 | CCCGCCGAGC AGGAGCGGCA TGGTCTGGTG CTGGTGGACG ACCTCAGCGA GTTTGACCTG |
| 5161 | GATGACATGA AAGCCGAGGG CCGGGAGCCT GCCCTGGTAG TGGAAACCAG CCCGAAGAAC |
| 5221 | TATCAGGCAT GGGTCAAGGT GGCCGACGCC GCAGGCGGTG AACTTCGGGG GCAGATTGCC |
| 5281 | CGGACGCTGG CCAGCGAGTA CGACGCCGAC CCGGCCAGCG CCGACAGCCG CCACTATGGC |
| 5341 | CGCTTGGCGG GCTTCACCAA CCGCAAGGAC AAGCACACCA CCCGCGCCGG TTATCAGCCG |
| 5401 | TGGGTGCTGC TGCGTGAATC CAAGGGCAAG ACCGCCACCG CTGGCCCGGC GCTGGTGCAG |
| 5461 | CAGGCTGGCC AGCAGATCGA GCAGGCCCAG CGGCAGCAGG AGAAGGCCCG CAGGCTGGCC |
| 5521 | AGCCTCGAAC TGCCCGAGCG GCAGCTTAGC CGCCACCGGC GCACGGCGCT GGACGAGTAC |
| 5581 | CGCAGCGAGA TGGCCGGGCT GGTCAAGCGC TTCGGTGATG ACCTCAGCAA GTGCGACTTT |
| 5641 | ATCGCCGCGC AGAAGCTGGC CAGCCGGGGC CGCAGTGCCG AGGAAATCGG CAAGGCCATG |
| 5701 | GCCGAGGCCA GCCCAGCGCT GGCAGAGCGC AAGCCCGGCC ACGAAGCGGA TTACATCGAG |
| 5761 | CGCACCGTCA GCAAGGTCAT GGGTCTGCCC AGCGTCCAGC TTGCGCGGGC CGAGCTGGCA |
| 5821 | CGGGCACCGG CACCCCGCCA GCGAGGCATG GACAGGGGCG GGCCAGATTT CAGCATGTAG |
| 5881 | TGCTTGCGTT GGTACTCACG CCTGTTATAC TATGAGTACT CACGCACAGA AGGGGGTTTT |
| 5941 | ATGGAATACG AAAAAAGCGC TTCAGGGTCG GTCTACCTGA TCAAAAGTGA CAAGGGCTAT |
| 6001 | TGGTTGCCCG GTGGCTTTGG TTATACGTCA AACAAGGCCG AGGCTGGCCG CTTTTCAGTC |
| 6061 | GCTGATATGG CCAGCCTTAA CCTTGACGGC TGCACCTTGT CCTTGTTCCG CGAAGACAAG |
| 6121 | CCTTTCGGCC CCGGCAAGTT TCTCGGTGAC TGATATGAAA GACCAAAAGG ACAAGCAGAC |
| 6181 | CGGCGACCTG CTGGCCAGCC CTGACGCTGT ACGCCAAGCG CGATATGCCG AGCGCATGAA |
| 6241 | GGCCAAAGGG ATGCGTCAGC GCAAGTTCTG GCTGACCGAC GACGAATACG AGGCGCTGCG |
| 6301 | CGAGTGCCTG GAAGAACTCA GAGCGGCGCA GGGCGGGGGT AGTGACCCCG CCAGCGCCTA |
| 6361 | ACCACCAACT GCCTGCAAAG GAGGCAATCA ATGGCTACCC ATAAGCCTAT CAATATTCTG |
| 6421 | GAGGCGTTCG CAGCAGCGCC GCCACCGCTG GACTACGTTT TGCCCAACAT GGTGGCCGGT |
| 6481 | ACGGTCGGGG CGCTGGTGTC GCCCGGTGGT GCCGGTAAAT CCATGCTGGC CCTGCAACTG |
| 6541 | GCCGCACAGA TTGCAGGCGG GCCGGATCTG CTGGAGGTGG GCGAACTGCC CACCGGCCCG |
| 6601 | GTGATCTACC TGCCCGCCGA AGACCCGCCC ACCGCCATTC ATCACCGCCT GCACGCCCTT |
| 6661 | GGGGCGCACC TCAGCGCCGA GGAACGGCAA GCCGTGGCTG ACGGCCTGCT GATCCAGCCG |
| 6721 | CTGATCGGCA GCCTGCCCAA CATCATGGCC CCGGAGTGGT TCGACGGCCT CAAGCGCGCC |
| 6781 | GCCGAGGGCC GCCGCCTGAT GGTGCTGGAC ACGCTGCGCC GGTTCCACAT CGAGGAAGAA |
| 6841 | AACGCCAGCG GCCCCATGGC CCAGGTCATC GGTCGCATGG AGGCCATCGC CGCCGATACC |
| 6901 | GGGTGCTCTA TCGTGTTCCT GCACCATGCC AGCAAGGGCG CGGCCATGAT GGGCGCAGGC |
| 6961 | GACCAGCAGC AGGCCAGCCG GGGCAGCTCG GTACTGGTCG ATAACATCCG CTGGCAGTCC |
| 7021 | TACCTGTCGA GCATGACCAG CGCCGAGGCC GAGGAATGGG GTGTGGACGA CGACCAGCGC |
| 7081 | CGGTTCTTCG TCCGCTTCGG TGTGAGCAAG GCCAACTATG GCGCACCGTT CGCTGATCGG |
| 7141 | TGGTTCAGGC GGCATGACGG CGGGGTGCTC AAGCCCGCCG TGCTGGAGAG GCAGCGCAAG |
| 7201 | AGCAAGGGGG TGCCCCGTGG TGAAGCCTAA GAACAAGCAC AGCCTCAGCC ACGTCCGGCA |
| 7261 | CGACCCGGCG CACTGTCTGG CCCCCGGCCT GTTCCGTGCC CTCAAGCGGG GCGAGCGCAA |
| 7321 | GCGCAGCAAG CTGGACGTGA CGTATGACTA CGGCGACGGC AAGCGGATCG AGTTCAGCGG |
| 7381 | CCCGGAGCCG CTGGGCGCTG ATGATCTGCG CATCCTGCAA GGGCTGGTGG CCATGGCTGG |
| 7441 | GCCTAATGGC CTAGTGCTTG GCCCGGAACC CAAGACCGAA GGCGGACGGC AGCTCCGGCT |
| 7501 | GTTCCTGGAA CCCAAGTGGG AGGCCGTCAC CGCTGAATGC CATGTGGTCA AAGGTAGCTA |
| 7561 | TCGGGCGCTG GCAAAGGAAA TCGGGGCAGA GGTCGATAGT GGTGGGGCGC TCAAGCACAT |
| 7621 | ACAGGACTGC ATCGAGCGCC TTTGGAAGGT ATCCATCATC GCCCAGAATG GCCGCAAGCG |
| 7681 | GCAGGGGTTT CGGCTGCTGT CGGAGTACGC CAGCGACGAG GCGGACGGGC GCCTGTACGT |
| 7741 | GGCCCTGAAC CCCTTGATCG CGCAGGCCGT CATGGGTGGC GGCCAGCATG TGCGCATCAG |
| 7801 | CATGGACGAG GTGCGGGCGC TGGACAGCGA AACCGCCCGC CTGCTGCACC AGCGGCTGTG |
| 7861 | TGGCTGGATC GACCCCGGCA AAACCGGCAA GGCTTCCATA GATACCTTGT GCGGCTATGT |
| 7921 | CTGGCCGTCA GAGGCCAGTG GTTCGACCAT GCGCAAGCGC CGCCAGCGGG TGCGCGAGGC |
| 7981 | GTTGCCGGAG CTGGTCGCGC TGGGCTGGAC GGTAACCGAG TTCGCGGCGG GCAAGTACGA |
| 8041 | CATCACCCGG CCCAAGGCGG CAGGCTGACC CCCCCCACTC TATTGTAAAC AAGACATTTT |
| 8101 | TATCTTTTAT ATTCAATGGC TTATTTTCCT GCTAATTGGT AATACCATGA AAAATACCAT |
| 8161 | GCTCAGAAAA GGCTTAACAA TATTTTGAAA AATTGCCTAC TGAGCGCTGC CGCACAGCTC |
| 8221 | CATAGGCCGC TTTCCTGGCT TTGCTTCCAG ATGTATGCTC TTCTGCTCCG ATCTGCGGGC |
| 8281 | AGTGAGCGCA ACGCAATTAA TGTGAGTTAG CTCACTCATT AGGCACCCCA GGCTTTACAC |
| 8341 | TTTATGCTTC CGGCTCGTAT AATGTGTGGA ATTGTGAGCG GATAACAATT TCACACAGGA |
| 8401 | TCTAGAAATA ATTTTGTTTA ACTTTAAGAA GGAGATATAC ATATATGAAA CCAGTAACGT |
| 8461 | TATACGATGT CGCAGAGTAT GCCGGTGTCT CTTATCAGAC CGTTTCCCGC GTGGTGAACC |
| 8521 | AGGCCAGCCA CGTTTCTGCG AAAACGCGGG AAAAAGTGGA AGCGGCGATG GCGGAGCTGA |
| 8581 | ATTACATTCC CAACCGCGTG GCACAACAAC TGGCGGGCAA ACAGTCGTTG CTGATTGGCG |
| 8641 | TTGCCACCTC CAGTCTGGCC CTGCACGCGC CGTCGCAAAT TGTCGCGGCG ATTAAATCTC |
| 8701 | GCGCCGATCA ACTGGGTGCC AGCGTGGTGG TGTCGATGGT AGAACGAAGC GGCGTCGAAG |
| 8761 | CCTGTAAAGC GGCGGTGCAC AATCTTCTCG CGCAACGCGT CAGTGGGCTG ATCATTAACT |
| 8821 | ATCCGCTGGA TGACCAGGAT GCCATTGCTG TGGAAGCTGC CTGCACTAAT GTTCCGGCGT |
| 8881 | TATTTCTTGA TGTCTCTGAC CAGACACCCA TCAACAGTAT TATTTTCTCC CATGAAGACG |
| 8941 | GTACGCGACT GGGCGTGGAG CATCTGGTCG CATTGGGTCA CCAGCAAATC GCGCTGTTAG |
| 9001 | CGGGCCCATT AAGTTCTGTC TCGGCGCGTC TGCGTCTGGC TGGCTGGCAT AAATATCTCA |
| 9061 | CTCGCAATCA AATTCAGCCG ATAGCGGAAC GGGAAGGCGA CTGGAGTGCC ATGTCCGGTT |
| 9121 | TTCAACAAAC CATGCAAATG CTGAATGAGG GCATCGTTCC CACTGCGATG CTGGTTGCCA |
| 9181 | ACGATCAGAT GGCGCTGGGC GCAATGCGCG CCATTACCGA GTCCGGGCTG CGCGTTGGTG |
| 9241 | CGGATATCTC GGTAGTGGGA TACGACGATA CCGAAGACAG CTCATGTTAT ATCCCGCCGT |
| 9301 | TAACCACCAT CAAACAGGAT TTTCGCCTGC TGGGGCAAAC CAGCGTGGAC CGCTTGCTGC |
| 9361 | AACTCTCTCA GGGCCAGGCG GTGAAGGGCA ATCAGCTGTT GCCCGTCTCA CTGGTGAAAA |
| 9421 | GAAAAACCAC CCTGGCGCCC AATACGCAAA CCGCCTCTCC CCGCGCGTTG GCCGATTCAT |
| 9481 | TAATGCAGCT GGCACGACAG GTTTCCCGAC TGGAAAGCGG GCAGTGAAAG CTGATCCGCG |
| 9541 | GCCGCCACGT TGTGTCTCAA AATCTCTGAT GTTACATTGC ACAAGATAAA AATATATCAT |
| 9601 | CATGAACAAT AAAACTGTCT GCTTACATAA ACAGTAATAC AAGGGGTGTT ATGAGCCATA |
| 9661 | TTCAACGGGA AACGTCTTGC TCGAGGCCGC GATTAAATTC CAACATGGAT GCTGATTTAT |
| 9721 | ATGGGTATAA ATGGGCTCGC GATAATGTCG GGCAATCAGG TGCGACAATC TATCGATTGT |
| 9781 | ATGGGAAGCC CGATGCGCCA GAGTTGTTTC TGAAACATGG CAAAGGTAGC GTTGCCAATG |
| 9841 | ATGTTACAGA TGAGATGGTC AGACTAAACT GGCTGACGGA ATTTATGCCT CTTCCGACCA |
| 9901 | TCAAGCATTT TATCCGTACT CCTGATGATG CATGGTTACT CACCACTGCG ATCCCCGGGA |
| 9961 | AAACAGCATT CCAGGTATTA GAAGAATATC CTGATTCAGG TGAAAATATT GTTGATGCGC |
| 10021 | TGGCAGTGTT CCTGCGCCGG TTGCATTCGA TTCCTGTTTG TAATTGTCCT TTTAACAGCG |
| 10081 | ATCGCGTATT TCGTCTCGCT CAGGCGCAAT CACGAATGAA TAACGGTTTG GTTGATGCGA |
| 10141 | GTGATTTTGA TGACGAGCGT AATGGCTGGC CTGTTGAACA AGTCTGGAAA GAAATGCATA |
| 10201 | AGCTTTTGCC ATTCTCACCG GATTCAGTCG TCACTCATGG TGATTTCTCA CTTGATAACC |
| 10261 | TTATTTTTGA CGAGGGGAAA TTAATAGGTT GTATTGATGT TGGACGAGTC GGAATCGCAG |
| 10321 | ACCGATACCA GGATCTTGCC ATCCTATGGA ACTGCCTCGG TGAGTTTTCT CCTTCATTAC |
| 10381 | AGAAACGGCT TTTTCAAAAA TATGGTATTG ATAATCCTGA TATGAATAAA TTGCAGTTTC |
| 10441 | ATTTGATGCT CGATGAGTTT TTCTAATCAG AATTGGTTAA TTGGTTGTAG GGATAACAGG |
| 10501 | GTAATTCTAG AGTCGACCTG CAGGCATGCA AGCTTAGATC CTTTGCCTGG CGGCAGTAGC |
| 10561 | GCGGTGGTCC CACCTGACCC CATGCCGAAC TCAGAAGTGA AACGCCGTAG CGCCGATGGT |
| 10621 | AGTGTGGGGT CTCCCCATGC GAGAGTAGGG AACTGCCAGG CATCAAATAA AACGAAAGGC |
| 10681 | TCAGTCGAAA GACTGGGCCT TTCGTTTTAT CTGTTGTTTG TCGGTGAACG CTCTCCTGAG |
| 10741 | TAGGACAAAT CCGCCGGGAG CGGATTTGAA CGTTGCGAAG CAACGGCCCG GAGGGTGGCG |
| 10801 | GGCAGGACGC CCGCCATAAA CTGCCAGGCA TCAAATTAAG CAGAAGGCCA TCCTGACGGA |
| 10861 | TGGCCTTTTT GCGTTTCTAC AAACTCTTTT TG |
| // | |

Plasmid Sequence for SSB Protein:

| | |
|---|---|
| LOCUS | p15a_Tet_SXTGamB 5308 bp ds-DNA circular |
| DEFINITION | |
| ACCESSION | |
| VERSION | |
| KEYWORDS | |
| SOURCE | |
| ORGANISM | |
| REFERENCE | |
| AUTHORS | Lee H., Ostrov N., Church G. |
| TITLE | |
| JOURNAL | UNPUBLISHED |
| PUBMED | |
| REFERENCE | |
| AUTHORS | |
| JOURNAL | |
| COMMENT | |
| FEATURES | Location/Qualifiers |
| source | 24 . . . 762 |
| | /organism = “synthetic DNA construct” |
| | /lab_host = “Escherichia coli” |
| | /mol_type = “other DNA” |
| | /ApEinfo_fwdcolor = “#1fff00” |
| | /ApEinfo_revcolor = “green” |
| | /ApEinfo_graphicformat = “arrow_data {{0 1 2 0 0 −1}{ } 01” |
| | /label = “source:synthetic DNA construct” |
| CDS | complement(30 . . . 653) |
| | /codon_start = 1 |
| | /gene = “tetR from transposon Tn10” |
| | /product = “tetracycline repressor TetR” |
| | /note = “TetR” |
| | /note = “TetR binds to the tetracycline operator tetO to |
| | inhibit transcription. This inhibition can be relieved by |
| | adding tetracycline or doxycycline.” |
| | /translation = “MSRLDKSKVINSALELLNEVGIEGLTTRKLAQKLGVEQPTLYWH |
| | VKNKRALLDALAIEMLDRHHTHFCPLEGESWQDFLRNNAKSFRCALLSHRDGAKVHLG |
| | TRPTEKQYETLENQLAFLCQQGFSLENALYALSAVGHFTLGCVLEDQEHQVAKEERET |
| | PTTDSMPPLLRQAIELFDHQGAEPAFLFGLELIICGLEKQLKCESGS” |
| | /ApEinfo_fwdcolor = “cyan” |
| | /ApEinfo_revcolor = “green” |
| | /ApEinfo_graphicformat = “arrow_data {{0 1 2 0 0 −1}{ } 01” |
| | /label = “tetracycline repressor TetR” |
| promoter | 672 . . . 727 |
| | /gene = “tetR” |
| | /note = “tetR/tetA promoters” |
| | /note = “overlapping promoters for bacterial tetR and tetA” |
| | /ApEinfo_fwdcolor = “#e900ff” |
| | /ApEinfo_revcolor = “green” |
| | /ApEinfo_graphicformat = “arrow_data {{0 1 2 0 0 −1}{ } 01” |
| | /label = “tetR” |
| protein_bind | 708 . . . 726 |
| | /gene = “tetO” |
| | /bound_moiety = “tetracycline repressor TetR” |
| | /note = “tet operator” |
| | /note = bacterial operator O2 for the tetR and tetA genes” |
| | /ApEinfo_fwdcolor = “pink” |
| | /ApEinfo_revcolor = “pink” |
| | /ApEinfo_graphicformat = “arrow_data {{0 1 2 0 0 −1}{ } 01” |
| | /label = “tetO” |
| RBS | 745 . . . 756 |
| | /note = “strong bacterial ribosome binding site (Elowitz |
| | andLeibler, 2000)” |
| | /label = “strong bacterial ribosome binding site (Elowitz |
| | and” |
| source | 763 . . . 1184 |
| | /organism = “Red-recombineering helper plasmid RSFRedkan” |
| | /mol_type = “other DNA” |
| | /db_xref = “taxon:570157” |
| | /ApEinfo_fwdcolor = “cyan” |
| | /ApEinfo_revcolor = “green” |
| | /ApEinfo_graphicformat = “arrow_data {{0 1 2 0 0 −1}{ } 01” |
| | /label = “source:Red-recombineering helper plasmid |
| | RSFRedkan” |
| gene | 763 . . . 1179 |
| | /gene = “gam” |
| | /ApEinfo_fwdcolor = “pink” |
| | /ApEinfo_revcolor = “pink” |
| | /ApEinfo_graphicformat = “arrow_data {{0 1 2 0 0 −1}{ } 01” |
| | /label = “gam” |
| CDS | 763 . . . 1179 |
| | /gene = “gam” |
| | /note = “derived from Escherichia coli lambda phage” |
| | /codon_start = 1 |
| | /transl_table = 11 |
| | /product = “Gam” |
| | /protein_id = “ACJ06683.1” |
| | /db_xref = “GI: 210076662” |
| | /translation = “MDINTETEIKQKHSLTPFPVFLISPAFRGRYFHSYFRSSAMNAY |
| | YIQDRLEAQSWARHYQQLAREEKEAELADDMEKGLPQHLFESLCIDHLQRHGASKKSI |
| | TRAFDDDVEFQERMAEHIRYMVETIAHHQVDIDSEV” |
| | /ApEinfo_fwdcolor = “cyan” |
| | /ApEinfo_revcolor = “green” |
| | /ApEinfo_graphicformat = “arrow_data {{0 1 2 0 0 −1}{ } 01” |
| | /label = “Gam” |
| misc_feature | 1217 . . . 2035 |
| | /ApEinfo_fwdcolor = “cyan” |
| | /ApEinfo_revcolor = “green” |
| | /ApEinfo_graphicformat = “arrow_data {{0 1 2 0 0 −1}{ } 01” |
| | /label = “SXT_Beta” |
| misc_feature | 2048 . . . 2467 |
| | /ApEinfo_fwdcolor = “cyan” |
| | /ApEinfo_revcolor = “green” |
| | /ApEinfo_graphicformat = “arrow_data {{0 1 2 0 0 −1}{ } 01” |
| | /label = “SXT-ssb” |
| misc_feature | 2479 . . . 3495 |
| | /ApEinfo_fwdcolor = “cyan” |
| | /ApEinfo_revcolor = “green” |
| | /ApEinfo_graphicformat = “arrow_data {{0 1 2 0 0 −1}{ } 01” |
| | /label = “SXT-Exo” |
| terminator | 3496 . . . 3567 |
| | /note = “rrnB T1 terminator” |
| | /note = “transcription terminator T1 from the E. coli rrnB |
| | gene” |
| | /ApEinfo_fwdcolor = “cyan” |
| | /ApEinfo_revcolor = “green” |
| | /ApEinfo_graphicformat = “arrow_data {{0 1 2 0 0 −1}{ } 01” |
| terminator | 3583 . . . 3610 |
| | /note = “T7Te terminator” |
| | /note = phage T7 early transcription terminator” |
| | /ApEinfo_fwdcolor = “cyan” |
| | /ApEinfo_reycolor = “green” |
| | /ApEinfo_graphicformat = “arrow_data {{0 1 2 0 0 −1}{ } 01” |
| rep_origin | complement(3772 . . . 4317) |
| | /direction = LEFT |
| | /note = “p15A ori |
| | /note = “Plasmids containing the medium-copy-number p15A |
| | origin of replication can be propagated in E. coli cells |
| | that contain a second plasmid with the ColE1 origin.” |
| | /ApEinfo_fwdcolor = “pink” |
| | /ApEinfo_revcolor = “pink” |
| | /ApEinfo_graphicformat = “arrow_data {{0 1 2 0 0 −1}{ } 01” |
| | /label = “p15A ori |
| terminator | 4431 . . . 4525 |
| | /note = “lambda t0 terminator” |
| | /note = “transcription terminator from phage lambda” |
| | /ApEinfo_fwdcolor = “cyan” |
| | /ApEinfo_revcolor = “green” |
| | /ApEinfo_graphicformat = “arrow_data {{0 1 2 0 0 −1}{ } 01” |
| CDS | complement(4546 . . . 5205) |
| | /codon_start = 1 |
| | /gene = “cat” |
| | /product = “chloramphenicol acetyltransferase” |
| | /note = “CmR” |
| | /note = “confers resistance to chloramphenicol” |
| | /translation = “MEKKITGYTTVDISQWHRKEHFEAFQSVAQCTYNQTVQLDITAF |
| | LKTVKKNKHKFYPAFIHILARLMNAHPEFRMAMKDGELVIWDSVHPCYTVFHEQTETF |
| | SSLWSEYHDDFRQFLHIYSQDVACYGENLAYFPKGFIENMFFVSANPWVSFTSFDLNV |
| | ANMDNFFAPVFTMGKYYTQGDKVLMPLAIQVHHAVCDGFHVGRMLNELQQYCDEWQGG |
| | A” |
| | /ApEinfo_fwdcolor = “cyan” |
| | /ApEinfo_revcolor = “green” |
| | /ApEinfo_graphicformat = “arrow_data {{0 1 2 0 0 −1} { } 0}” |
| | /label = “chloramphenicol acetyltransferase” |
| promoter | complement(5206 . . . 5308) |
| | /note = “cat promoter” |
| | /note = “promoter of the E. coli cat gene encoding |
| | chloramphenicol acetyltransferase” |
| | /ApEinfo_fwdcolor = “#e900ff” |
| | /ApEinfo_revcolor = “green” |
| | /ApEinfo_graphicformat = “arrow_data {{0 1 2 0 0 −1} { } 0}” |
| ORIGIN | |

| 1 | acgtctcatt ttcgccagat atcgacgtct taagacccac tttcacattt aagttgtttt |
|---|---|
| 61 | tctaatccgc atatgatcaa ttcaaggccg aataagaagg ctggctctgc accttggtga |
| 121 | tcaaataatt cgatagcttg tcgtaataat ggcggcatac tatcagtagt aggtgtttcc |
| 181 | ctttcttctt tagcgacttg atgctcttga tcttccaata cgcaacctaa agtaaaatgc |
| 241 | cccacagcgc tgagtgcata taatgcattc tctagtgaaa aaccttgttg gcataaaaag |
| 301 | gctaattgat tttcgagagt ttcatactgt ttttctgtag gccgtgtacc taaatgtact |
| 361 | tttgctccat cgcgatgact tagtaaagca catctaaaac ttttagcgtt attacgtaaa |
| 421 | aaatcttgcc agctttcccc ttctaaaggg caaaagtgag tatggtgcct atctaacatc |
| 481 | tcaatggcta aggcgtcgag caaagcccgc ttatttttta catgccaata caatgtaggc |
| 541 | tgctctacac ctagcttctg ggcgagttta cgggttgtta aaccttcgat tccgacctca |
| 601 | ttaagcagct ctaatgcgct gttaatcact ttacttttat ctaatctaga catcattaat |
| 661 | tcctaatttt tgttgacact ctatcgttga tagagttatt ttaccactcc ctatcagtga |
| 721 | tagagaaaag aattcaaaag atctaaagag gagaaaggat ctatggatat taatactgaa |
| 781 | actgagatca agcaaaagca ttcactaacc ccctttcctg ttttcctaat cagcccggca |
| 841 | tttcgcgggc gatattttca cagctatttc aggagttcag ccatgaacgc ttattacatt |
| 901 | caggatcgtc ttgaggctca gagctgggcg cgtcactacc agcagctcgc ccgtgaagag |
| 961 | aaagaggcag aactggcaga cgacatggaa aaaggcctgc cccagcacct gtttgaatcg |
| 1021 | ctatgcatcg atcatttgca acgccacggg gccagcaaaa aatccattac ccgtgcgttt |
| 1081 | gatgacgatg ttgagtttca ggagcgcatg gcagaacaca tccggtacat ggttgaaacc |
| 1141 | attgctcacc accaggttga tattgattca gaggtataaa acgagcagac tcccactttg |
| 1201 | attgtcggag tccacaatgg aaaaaccaaa gctaatccaa cgctttgctg agcgctttag |
| 1261 | tgtcgatcca aacaaactgt tcgataccct aaaagcaaca gcatttaagc aacgtgatgg |
| 1321 | tagtgcaccg accaatgagc agatgatggc gctcttggtg gttgcagatc agtacggctt |
| 1381 | gaaccctttc accaaagaga tttttgcgtt ccctgataag caagctggaa ttattccagt |
| 1441 | ggtaggtgtc gatggatggt ctcgcatcat caatcaacac gaccagtttg atggcatgga |
| 1501 | gtttaagact tcagaaaaca aagtctccct ggatggcgcg aaagaatgcc cggaatggat |
| 1561 | ggaatgcatt atctaccggc gcgaccgttc gcacccagtc aaaatcactg aatacctgga |
| 1621 | tgaagtctat cgaccgcctt ttgagggtaa cggaaaaaat ggcccttacc gtgtagatgg |
| 1681 | tccatggcag acgcacacta agcgaatgct aagacataaa tccatgatcc agtgttcccg |
| 1741 | cattgcgttt ggctttgtgg gaattttcga tcaagacgaa gcggagcgaa ttatcgaagg |
| 1801 | ccaagcaaca cacattgttg agccatcggt gattccaccc gagcaagttg atgatcgaac |
| 1861 | ccgagggctt gtttacaagc ttatcgagcg ggcggaagct tcaaacgcat ggaatagtgc |
| 1921 | attggaatac gccaatgaac attttcaagg tgttgaactg acgtttgcga aacaagaaat |
| 1981 | atttaatgca cagcaacaag cagccaaagc gctcacacag cctttagctt cttagctcga |
| 2041 | gtaaggaatg aaaaaccaag taacactcat aggctatgtt ggctctgagc cagagacgcg |
| 2101 | agcctatcca tcaggtgatt tagtgaccag catttcactg gccacttctg agaaatggcg |
| 2161 | cgaccgtcaa tccaatgagc tcaaagagca tacggaatgg catcgggtcg tttttcgaga |
| 2221 | tcgtggtgga ttaaagttag ggctcagggc aaaagattta atccaaaaag gagcgaagct |
| 2281 | ttttgttcaa gggcctcagc gcacgcgctc atgggagaaa gatggcatta agcatcgatt |
| 2341 | gaccgaagtg gacgcggacg agtttctgct tcttgataat gtgaacaaag catctgagcc |
| 2401 | atcagcggcg gatgatgcag gctcccaaac taattgggca caaacttatc ctgaaccaga |
| 2461 | tttttaatct ccaggcatat gaaggttatc gacctatcac aacgtactcc tgcatggcac |
| 2521 | cagtggcgca ttgcaggggt tacggcatct gaagccccaa ttattatggg gcgttcaccc |
| 2581 | tacaaaacac cttggcgatt atgggcagaa aaaactggat tcgtattacc ggaagacctg |
| 2641 | tcgaataatc ctaatgtact tcgcggtata aggttggagc ctcaagcaag gcgagcattt |
| 2701 | gagaatgcgc ataatgactt tcttctgccg ttatgtgcag aagccgatca taacgcaatc |
| 2761 | tttcgagcca gctttgatgg catcaacgat gcgggcgagc ccgttgaact gaaatgtcct |
| 2821 | tgccagtcag tttttgagga tgtgcaagct caccgagaac aaagcgaggc gtaccagttg |
| 2881 | tattgggtgc aagtacagca tcaaatactg gtcgccaata gcacgcgagg ttggttggtt |
| 2941 | ttctattttg aggatcaact gattgagttt gaaatacaac gagacgcggc gttcttaact |
| 3001 | gagttgcaag aaacagcgct tcagttttgg gagttagtac agaccaaaaa agaaccgtca |
| 3061 | aaatgccctg agcaagattg ttttgttccc aagggtgaag cccaataccg ttggacatcg |
| 3121 | ctgtctcgac agtattgctc agcacatgcc gaagtggtcc gactggaaaa tcacattaaa |
| 3181 | tctttgaaag aggaaatgcg agacgctcag tcaaaattgg tcgccatgat gggtaactac |
| 3241 | gctcatgccg actatgctgg ggtcaaactc agtcgctaca tgatggcggg cacggtggac |
| 3301 | tataagcaat tggccaccga taaattaggc gagctggatg aacaggtttt agccgcttac |
| 3361 | cgaaaagcgc cacaagagcg gttgcgtatc agcaccaata agccagagca gcccgttgaa |
| 3421 | acaccaatca aaatcagcct tgagcaagag aacttggttc tgccaggtga ctcgccgagc |
| 3481 | tcattttatt tttaacaaat aaaacgaaag gctcagtcga aagactgggc ctttcgtttt |
| 3541 | atctgttgtt tgtcggtgaa cgctctctac tagagtcaca ctggctcacc ttcgggtggg |
| 3601 | cctttctgcg tttataccta gggatatatt ccgcttcctc gctcactgac tcgctacgct |
| 3661 | cggtcgttcg actgcggcga gcggaaatgg cttacgaacg gggcggagat ttcctggaag |
| 3721 | atgccaggaa gatacttaac agggaagtga gagggccgcg gcaaagccgt ttttccatag |
| 3781 | gctccgcccc cctgacaagc atcacgaaat ctgacgctca aatcagtggt ggcgaaaccc |
| 3841 | gacaggacta taaagatacc aggcgtttcc ccctggcggc tccctcgtgc gctctcctgt |
| 3901 | tcctgccttt cggtttaccg gtgtcattcc gctgttatgg ccgcgtttgt ctcattccac |
| 3961 | gcctgacact cagttccggg taggcagttc gctccaagct ggactgtatg cacgaacccc |
| 4021 | ccgttcagtc cgaccgctgc gccttatccg gtaactatcg tcttgagtcc aacccggaaa |
| 4081 | gacatgcaaa agcaccactg gcagcagcca ctggtaattg atttagagga gttagtcttg |
| 4141 | aagtcatgcg ccggttaagg ctaaactgaa aggacaagtt ttggtgactg cgctcctcca |
| 4201 | agccagttac ctcggttcaa agagttggta gctcagagaa ccttcgaaaa accgccctgc |
| 4261 | aaggcggttt tttcgttttc agagcaagag attacgcgca gaccaaaacg atctcaagaa |
| 4321 | gatcatctta ttaatcagat aaaatatttc tagatttcag tgcaatttat ctcttcaaat |
| 4381 | gtagcacctg aagtcagccc catacgatat aagttgttac tagtgcttgg attctcacca |
| 4441 | ataaaaaacg cccggcggca accgagcgtt ctgaacaaat ccagatggag ttctgaggtc |
| 4501 | attactggat ctatcaacag gagtccaagc gagctcgata tcaaattacg ccccgccctg |
| 4561 | ccactcatcg cagtactgtt gtaattcatt aagcattctg ccgacatgga agccatcaca |
| 4621 | aacggcatga tgaacctgaa tcgccagcgg catcagcacc ttgtcgcctt gcgtataata |
| 4681 | tttgcccatg gtgaaaacgg gggcgaagaa gttgtccata ttggccacgt ttaaatcaaa |
| 4741 | actggtgaaa ctcacccagg gattggctga gacgaaaaac atattctcaa taaacccttt |
| 4801 | agggaaatag gccaggtttt caccgtaaca cgccacatct tgcgaatata tgtgtagaaa |
| 4861 | ctgccggaaa tcgtcgtggt attcactcca gagcgatgaa aacgtttcag tttgctcatg |
| 4921 | gaaaacggtg taacaagggt gaacactatc ccatatcacc agctcaccgt ctttcattgc |
| 4981 | catacgaaat tccggatgag cattcatcag gcgggcaaga atgtgaataa aggccggata |
| 5041 | aaacttgtgc ttatttttct ttacggtctt taaaaaggcc gtaatatcca gctgaacggt |
| 5101 | ctggttatag gtacattgag caactgactg aaatgcctca aaatgttctt tacgatgcca |
| 5161 | ttgggatata tcaacggtgg tatatccagt gatttttttc tccattttag cttccttagc |
| 5221 | tcctgaaaat ctcgataact caaaaaatac gcccggtagt gatcttattt cattatggtg |
| 5281 | aaagttggaa cctcttacgt gccgatca |
| // | |

### Example VI

### CRISPR Mediated Target Gene Silencing in Vibrio natriegens

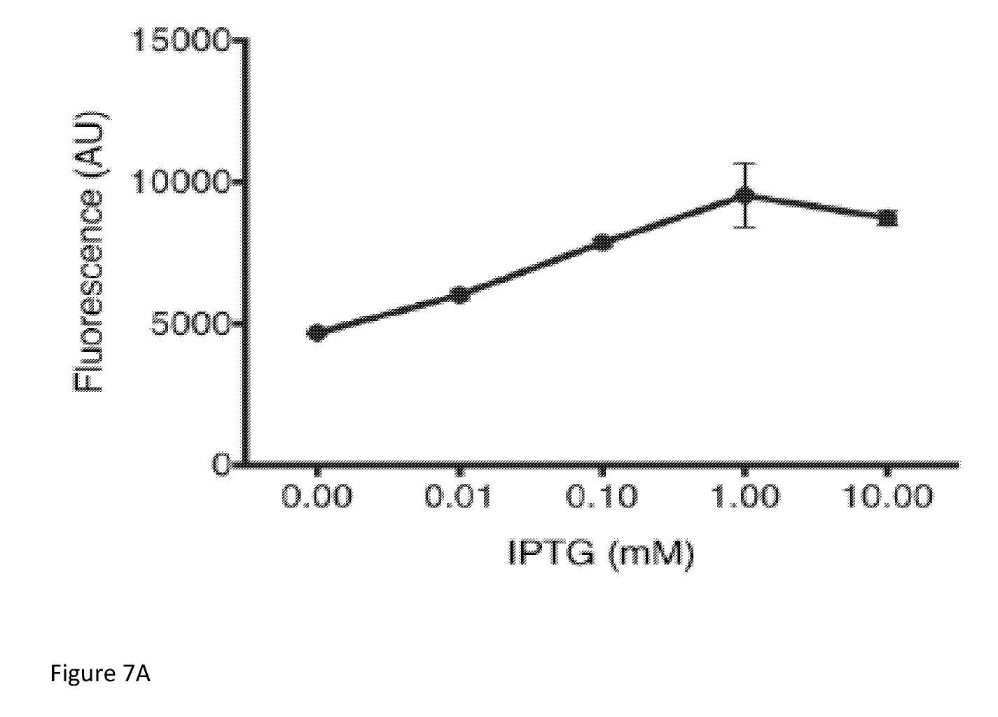

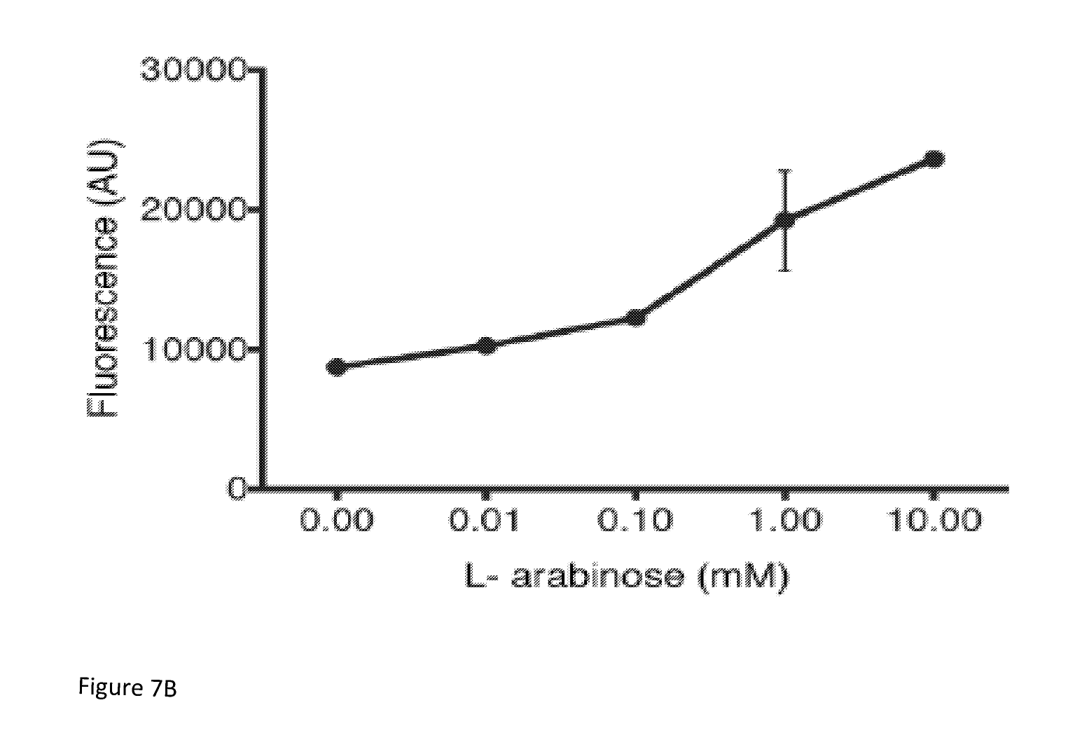

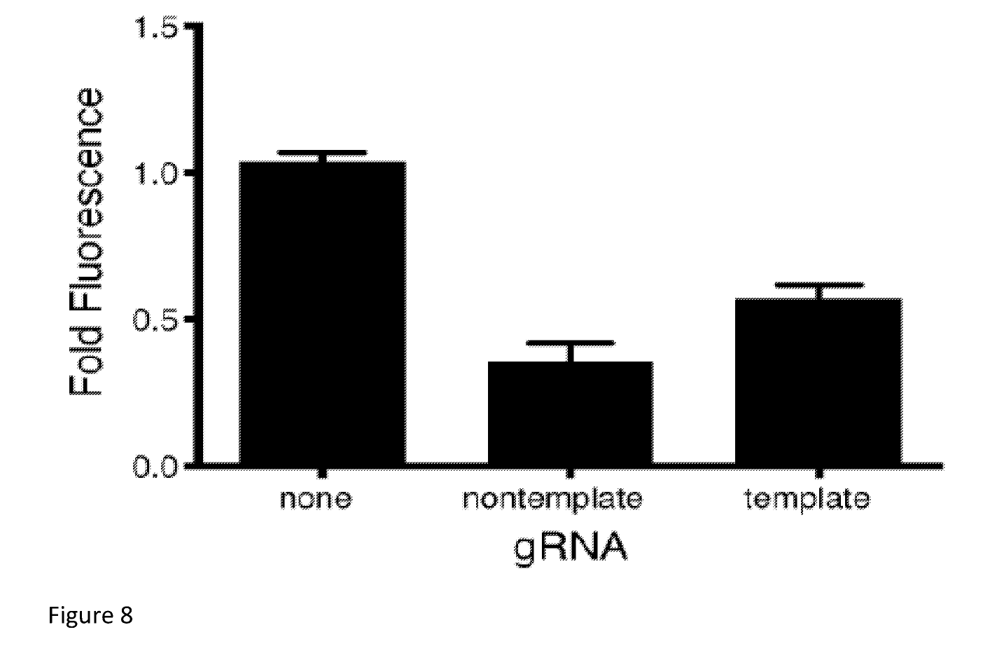

CRISPRi is capable of targeted gene inhibition but requires a genetic system capable of controlled expression with a measurable phenotype (See, e.g., Qi, Lei S., Matthew H. Larson, Luke A. Gilbert, Jennifer A. Doudna, Jonathan S. Weissman, Adam P. Arkin, and Wendell A. Lim. 2013. “Repurposing CRISPR as an RNAGuided Platform for Sequence Specific Control of Gene Expression.” Cell 152 (5): 1173-83, hereby incorporated by reference in its entirety). To develop a CRISPRi system in Vibrio natriegens, it was first established that the commonly used lactose and arabinose induction systems were operable, and characterized their dynamic ranges using GFP (FIGS. 7A-7B) (See, e.g., Jacob, F., and J. Monod. 1961. “On the Regulation of Gene Activity.” Cold Spring Harbor Symposia on Quantitative Biology 26 (0): 193-211; Schleif, R. 2000. “Regulation of the L-Arabinose Operon of Escherichia Coli.” Trends in Genetics: TIG 16 (12): 559-65, hereby incorporated in references in their entireties). The dCas9 was placed under the control of arabinose promoter and the guide RNA under the control of the constitutive promoter J23100. Next, a transposon system was used to genomically integrate a constitutively expressed GFP construct (as described in bioRxiv (Jun. 12, 2016) doi: http://dx.doi.org/10.1101/058487 hereby incorporated by reference in its entirety). Using this engineered reporter strain, it was shown that inducing dCas9 in the presence of guide RNAs significantly inhibits chromosomal expression of GFP. Consistent with previous studies, stronger inhibition was found when using a guide RNA that targets the nontemplate strand (FIG. 8) (Larson, Matthew H., Luke A. Gilbert, Wang Xiaowo, Wendell A. Lim, Jonathan S. Weissman, and Lei S. Qi. 2013. “CRISPR Interference (CRISPRi) for Sequence Specific Control of Gene Expression.” Nature Protocols 8 (11): 2180-96, hereby incorporated in reference in its entirety). The guide RNA sequences for the template (sense) and nontemplate (antisense) strand used are GAATTCATTAAAGAGGAGAA and TTTCTCCTCTTTAATGAATT, respectively. This embodiment of the present disclosure exemplifies a dCas9 mediated target gene inactivation in Vibrio natriegens, the scope of CRISPR mediated target nucleic acid sequence alteration or modulation of target gene expression should not be construed as so limited but should encompass all types of target nucleic acid sequence alteration including but not limited to insertion, deletion, and mutation, as well as target gene repression or activation using the CRISPR system in Vibrio natriegens according to techniques known to a skilled in the art. This example can be scaled for genome-wide perturbations in Vibrio natriegens according to techniques known to a skilled in the art (See, e.g., Peters, Jason M., Alexandre Colavin, Handuo Shi, Tomasz L. Czarny, Matthew H. Larson, Spencer Wong, John S. Hawkins, et al. 2016. “A Comprehensive, CRISPR Based Functional Analysis of Essential Genes in Bacteria.” Cell 165 (6): 1493-1506, hereby incorporated in reference in its entirety).

### Growth Media

Standardized growth media for Vibrio natriegens is named LB3 Lysogeny Broth with 3% (w/v) final NaCl. This media was prepared by adding 20 grams of NaCl to 25 grams of LB Broth Miller (Fisher BP9723500). Rich media were formulated according to manufacturer instructions and supplemented with 1.5% final Ocean Salts (Aquarium System, Inc.) (w/v) to make high salt versions of Brain Heart Infusion (BHIO), Nutrient Broth (NBO), and Lysogeny Broth (LBO). No additional salts were added to Marine Broth (MB). Minimal M9 media was prepared according to manufacturer instruction. For culturing Vibrio natriegens, 2% (w/v) final sodium chloride was added to M9. Carbon sources were added as indicated to 0.4% (v/v). Unless otherwise indicated, Vibrio natriegens experiments were performed in LB3 media and Escherichia coli experiments were performed in LB media. SOC3 media is composed of 5 grams of yeast extract, 20 grams tryptone, 30 grams sodium chloride, 2.4 grams magnesium sulfate, and 0.4% (v/v) final glucose.

### Overnight Culturing

An inoculation of 80° C. frozen stock of Vibrio natriegens can reach stationary phase after 5 hours when incubated at 37° C. Prolonged overnight culturing (>15 hours) at 37° C. can lead to an extended lag phase upon subculturing. Routine overnight culturing of Vibrio natriegens is performed for 815 hours at 37° C. or 12-24 hours at room temperature. Unless otherwise indicated, Escherichia coli cells used in this study were K12 subtype MG1655 unless otherwise indicated and cultured overnight (>10 hours) at 37° C. Vibrio cholerae 0395 was cultured overnight (>10 hours) in LB at 30° C. or 37° C. in a rotator drum at 150 rpm.

### Glycerol Stock

To prepare Vibrio natriegens cells for 80° C. storage, an overnight culture of cells must be washed in fresh media before storing in glycerol. A culture was centrifuged for 1 minute at 20,000 rcf and the supernatant was removed. The cell pellet was resuspended in fresh LB3 media and glycerol was added to 20% final concentration. The stock is quickly vortexed and stored at 80° C. Note: unlike glycerol stocks of Escherichia coli for 80° C. storage, neglecting the washing step prior to storing Vibrio natriegens cultures at 80° C. can lead to an inability to revive the culture.

### Plasmid Constructions

Routine cloning was performed by PCR of desired DNA fragments, assembly with NEB Gibson Assembly or NEBuilder HiFi DNA Assembly, and propagation in Escherichia coli (Gibson, Daniel, and Gibson Daniel. 2009. “OneStep Enzymatic Assembly of DNA Molecules up to Several Hundred Kilobases in Size.” Protocol Exchange. doi: 10.1038/nprot.2009.77, hereby incorporated by reference in its entirety) unless otherwise indicated. pRSF was used for the majority of this work since it carries all of its own replication machinery and should be minimally dependent on host factors (Katashkina, Joanna I., Hara Yoshihiko, Lyubov I. Golubeva, Irina G. Andreeva, Tatiana M. Kuvaeva, and Sergey V. Mashko. 2009. “Use of the λ RedRecombineering Method for Genetic Engineering of Pantoea Ananatis.” BMC Molecular Biology 10 (1): 34, hereby incorporated by reference in its entirety). For the transformation optimizations, pRSFpLtetOgfp was constructed, which constitutively expresses GFP due to the absence of the tetR repressor in both Escherichia coli and Vibrio natriegens. The pRST shuttle plasmid was engineered by fusing the pCTXKm replicon with the pirdependent conditional replicon, R6k. To construct the conjugative suicide mariner transposon, the Tn5 transposase and Tn5 mosaic ends were replaced in pBAM1 with the mariner C9 transposase and the mariner mosaic ends from pTnFGL3 (Cameron, D. Ewen, Jonathan M. Urbach, and John J. Mekalanos. 2008. “A Defined Transposon Mutant Library and Its Use in Identifying Motility Genes in Vibrio Cholerae.” Proceedings of the National Academy of Sciences of the United States of America 105 (25): 8736-41; MartínezGarcía, Esteban, Belën Calles, Miguel ArévaloRodríguez, and Victor de Lorenzo. 2011. “pBAM1: An All Synthetic Genetic Tool for Analysis and Construction of Complex Bacterial Phenotypes.” BMC Microbiology 11 (February): 38, hereby incorporated in references in their entireties). Our payload, the transposon DNA, consisted solely of the minimal kanamycin resistance gene required for transconjugant selection. Site directed mutagenesis were next performed on both transposon mosaic ends to introduce an MmeI cutsite, producing the plasmid pMarC9 which is also based on the pirdependent conditional replicon, R6k. A transposon plasmid capable of integrating a constitutively expressing GFP cassette in the genome by inserting pLtetOGFP with either kanamycin or spectinomycin in the transposon DNA was also constructed. All plasmids carrying the R6k origin was found only to replicate in either BW29427 or EC100D pir+/pir116 Escherichia coli cells. Induction systems were cloned onto the pRSF backbone. For the CRISPRi system, a single plasmid carrying both dCas9, the nuclease null Streptococcus pyogenes cas9, and the guide RNA was utilized. The dCas9 was under the control of arabinose induction and the guide RNA was under control of the constitutive J23100 promoter.

### Arabinose and IPTG Induction Assay

Vibrio natriegens carrying plasmid pRSFpBADGFP or pRSFpLacIGFP were used for all induction assays. Overnight cultures were washed with LB3 media and diluted 1:1000 into selective LB3 media with varying concentration of IPTG or Larabinose. OD600 and fluorescence were kinetically monitored in a microplate with orbital shaking at 37° C. Fluorescence after 7 hours of culturing is shown.

Repression of Chromosomally-Encoded GFP with CRISPRi

Our previously described transposon system was used to chromosomally integrated a cassette that constitutively expresses GFP. This engineered Vibrio natriegens strain was transformed with our CRISPRi plasmid carrying both dcas9 and GFP targeting gRNA. To test the repression of the chromosomally-encoded GFP with CRISPRi, the overnight cultures were subcultured 1:1000 in fresh media supplemented with or without 1 mM arabinose. OD600 and fluorescence of each culture were kinetically measured over 12 hours in a microplate with orbital shaking at 37° C. In these conditions, all cultures grew equivalently. Fold repression was calculated as the ratio of final fluorescence for each construct with or without the addition of arabinose.

### Example VII

### Methods for DNA Delivery in Vibrio natriegens

Vibrio natriegens is the fastest dividing free-living organism known, doubling >2 times faster than E. coli (H. H. Lee et al., “Vibrio natriegens, a new genomic powerhouse” (2016), doi: 10.1101/058487). Performing biological research or production with an ultrafast growth rate would significantly reduce time in the laboratory or in fermentors, most of which is spent waiting on cell growth. As such, V. natriegens has been proposed as an attractive next-generation microbial workhorse.

Delivery of circular or linear DNA into cells by electroporation has been demonstrated for several laboratory organisms, including E. coli (W. J. Dower, J. F. Miller, C. W. Ragsdale, High efficiency transformation of E. coli by high voltage electroporation. Nucleic Acids Res. 16, 6127-6145 (1988)), S. cerevisiae (D. M. Becker, L. Guarente, High-efficiency transformation of yeast by electroporation. Methods Enzymol. 194, 182-187 (1991)), plant cells (M. E. Fromm, L. P. Taylor, V. Walbot, Stable transformation of maize after gene transfer by electroporation. Nature. 319, 791-793 (1986)) mammalian cells (E. Neumann, M. Schaefer-Ridder, Y. Wang, P. H. Hofschneider, Gene transfer into mouse lyoma cells by electroporation in high electric fields. EMBO J. 1, 841-845 (1982), H. Aihara, J. Miyazaki, Gene transfer into muscle by electroporation in vivo. Nat. Biotechnol. 16, 867-870 (1998)) and other organisms. The efficiency of DNA transformation method is an important determinant of our ability to genetically manipulate and study an organism in the lab. Highly efficient transformation thus enables advanced applications such as high throughput library screens and genomic studies.

In this example, we establish the utility of DNA transformation method by electroporation into V. natriegens, and optimization of electroporation conditions. Specifically, as shown below, we demonstrate: 1) Transformation protocol for plasmid DNA into V. natriegens via electroporation; and 2) Optimization of electroporation conditions. These methods can be used for, including but not limited to: delivery of circular or linear recombinant DNA or libraries into V. natriegens for purposes of protein expression or genomic modification such as insertion or deletions.

Transformation Protocol for V. natriegens Recombination with Beta or s065 Recombinase Using Single-Stranded Oligonucleotides or Double-Stranded Cassette

Provided are procedures of the transformation protocol used herein.

1. Grow cultures overnight,

2. Subculture overnight cells in desired growth media,

3. Prepare electrocuvettes with up to 5 μL of DNA (>=50 μM of single-stranded DNA oligo and about 1 μg of double-stranded DNA oligo) and place on ice,

4. Wash the cells in 1M cold sorbitol, and concentrate cells 200× by volume,

5. Electroporate with the following settings: 0.4 kV, 1 kΩ, 25 μF; time constants should be >12 ms,

6. Quickly, recover the cells from the electrocuvette in rich media, and

7. Plate cells and incubate for colony formation.

Detailed electrotransformation protocol can be found in the BioRxiv paper (H. H. Lee et al., “Vibrio natriegens, a new genomic powerhouse” (2016), doi:10.1101/058487).

Optimization of the Protocol for Electroporation of Plasmids in V. natriegens

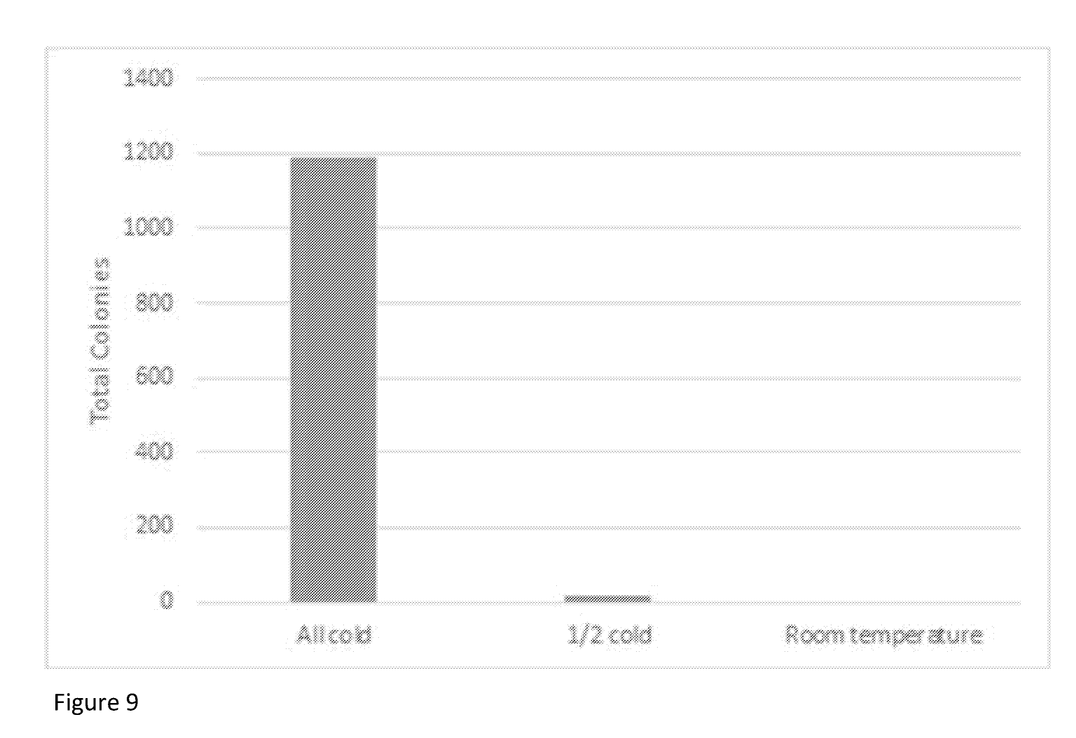

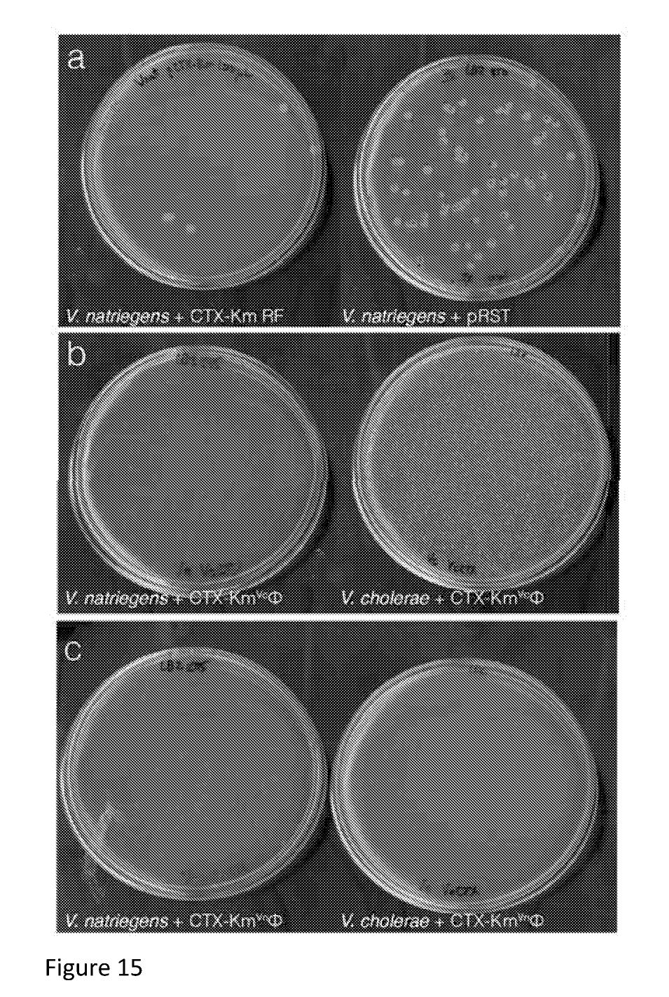

FIGS. 9 and 15 shows assays for optimization of the protocol for electroporation of plasmids in V. natriegens. These assays used a plasmid carrying a spectinomycin or carbenicillin resistance marker. Transformation efficiency was scored by counting the number of colonies resistant to the corresponding antibiotic used in the assay. All experiments were performed using pRSF plasmid as described in (H. H. Lee et al., “Vibrio natriegens, a new genomic powerhouse” (2016), doi:10.1101/058487). All experiments performed used 50 ng of pRSF plasmid unless indicated otherwise.

### Example VIII

Methods for Improving V. natriegens Growth Rate by Genome-Wide Pooled CRISPR Inhibition

This experiment discloses methods for improving V. natriegens growth rate by genome-wide pooled CRISPR inhibition. Vibrio natriegens is the fastest dividing bacteria (Weinstock, M. T., Hesek, E. D., Wilson, C. M. & Gibson, D. G. Vibrio natriegens as a fast-growing host for molecular biology. Nat. Methods 13, 849-851 (2016)), yet little is known about its biology (Lee, H. H. et al. Vibrio natriegens, a new genomic powerhouse. (2016). doi: 10.1101/058487, Dalia, T. N. et al. Multiplex Genome Editing by Natural Transformation (MuGENT) for Synthetic Biology in Vibrio natriegens. ACS Synth. Biol. (2017). doi: 10.1021/acssynbio.7b00116). The genetics underlying its record-setting growth rate was investigated. Generation time was quantified by single-cell imaging, and its most rapid growth was visualized at 37° C. By quantifying genome coverage of dividing cells, it was found that fast growth is not driven by an increase in DNA replication forks. Instead, translational regulation was found as the most significant determinant for rapid growth. Transcriptional profiling showed that ribosomal and protein biosynthesis pathways are the most significant differentially regulated processes across growth conditions, corroborated by the high copy numbers of tRNAs and rRNAs in the genome. High-efficiency transformation and CRISPR inhibition tools (CRISPRi) were established for V. natriegens and a 13,567-membered gRNA library was used to assess all protein-coding genes. 1070 genes essential for its record-setting growth rate were identified, comprising 604 genes critical for survival and 466 additional genes specifically required to maintain fast growth. Fast growth genes are uniquely enriched for sulfur metabolism and tRNA modifications, implicating a role for sulfur assimilation and translation efficiency in rapid cell division. The methods disclosed herein serve to advance fundamental V. natriegens biology and as foundation for further study and engineering of this unique organism.

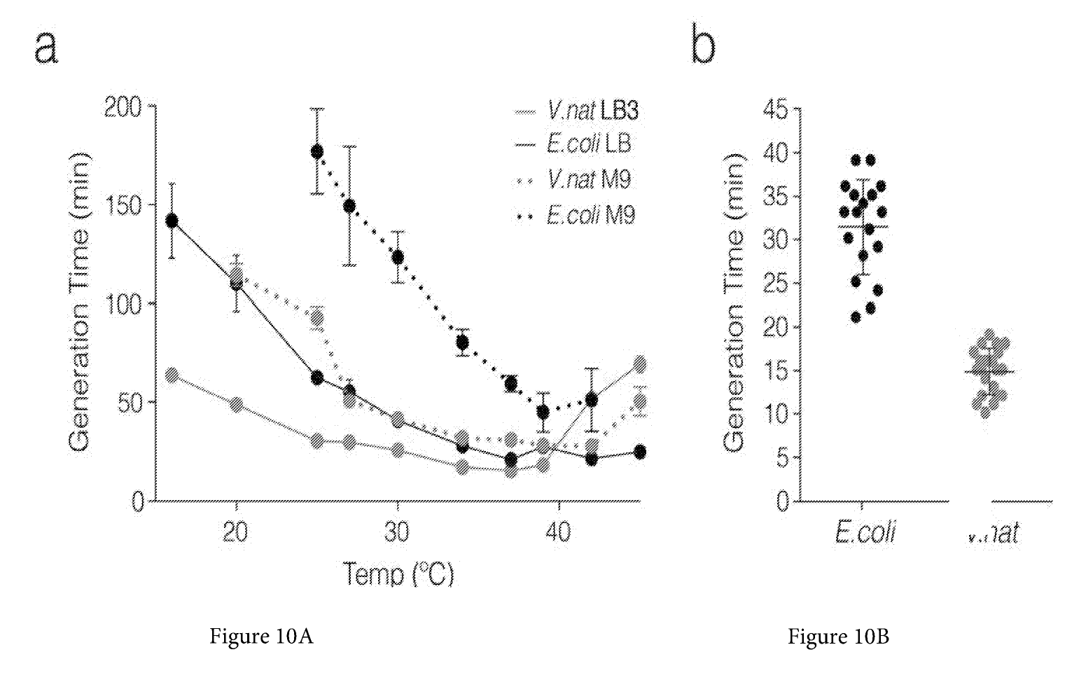

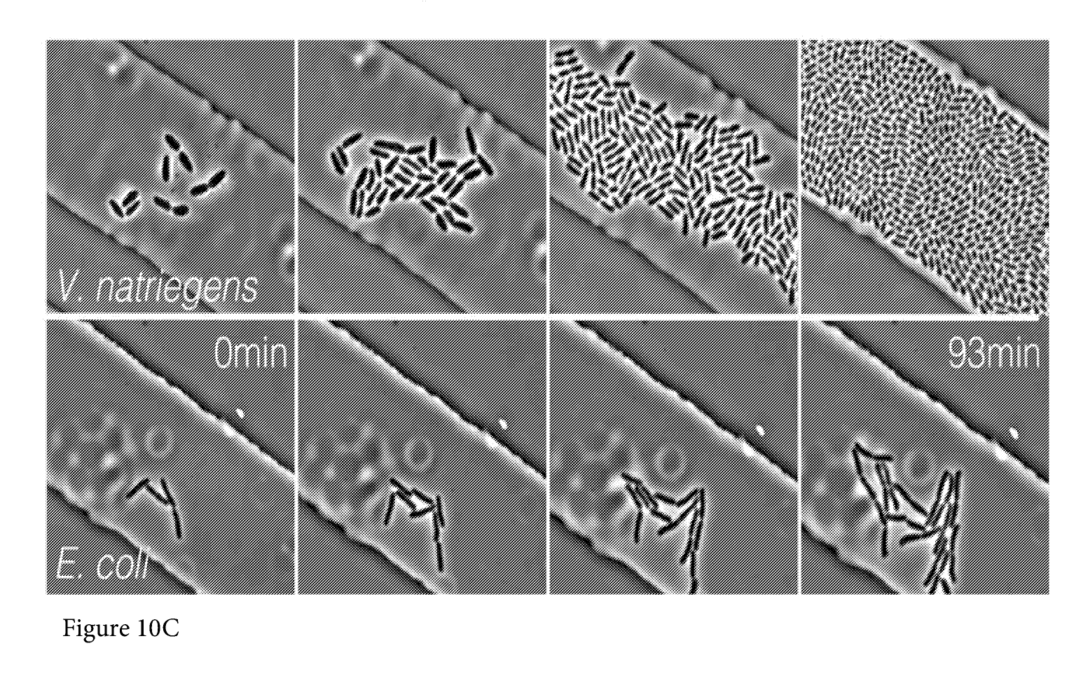

To investigate the genetics underlying its rapid growth, conditions for routine culturing was explored, aiming for readily-made, salt-rich media to support rapid and consistent growth. Lysogeny Broth supplemented with 3% (w/v) sodium chloride (LB3) was settled as our standard rich media due to the simplicity and accessibility of its formulation; commercial sea salts resulted in slightly faster growth but their compositions are complex and variable (Atkinson, M. J., and Bingham, C. Elemental composition of commercial seasalts. J. Aquaricult. Aquat. Sci. VIII, 39-43 (1997)). V. natriegens' generation time in bulk culture was quantified and it was found that it outpaced E. coli across all tested temperatures under 42° C.: 1.4-2.2 times faster in rich media and 1.6-3.9 times faster in minimal glucose media supplemented with salt (FIG. 10A). Generation time was further quantified by time-lapse, single-cell microscopy using custom microfluidic chemostats (FIGS. 10B-10C). It was found that V. natriegens generation time to be 14.8 minutes in LB3, 2.1 times faster than that of E. coli in LB (31.3 minutes).

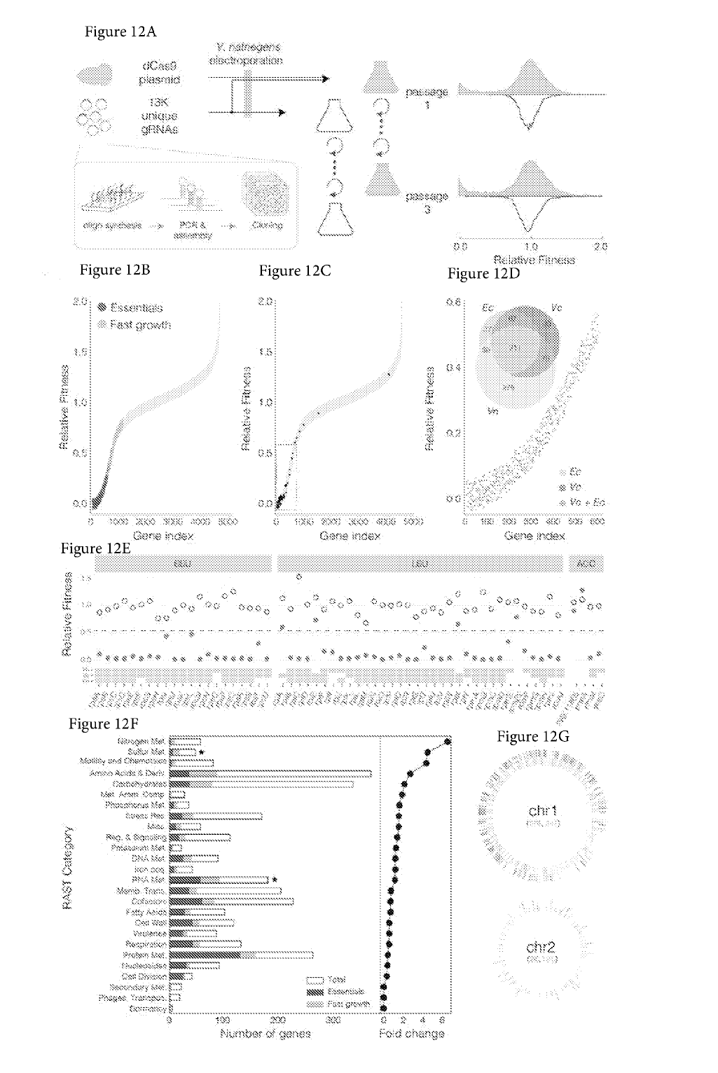

As basis for further genetic investigation, we produced the first de novo genome assembly of two closed fully annotated circular chromosomes of 3.24 Mb (chr1) and 1.92 Mb (chr2) (FIG. 12A; Table 1, Table 2, Methods, RefSeq NZ_CP009977-8) (H. H. Lee et al., “Vibrio natriegens, a new genomic powerhouse” (2016), doi:10.1101/058487). We found 36,599 putative methylated adenine residues at GATC motifs based on single molecule sequencing kinetics; Dam methylation has been previously shown in V. cholerae to be essential for stable chromosome replication (Julio, S. M. et al. DNA Adenine Methylase Is Essential for Viability and Plays a Role in the Pathogenesis of Yersinia pseudotuberculosis and Vibrio cholerae. Infect. Immun. 69, 7610-7615 (2001)). RAST annotation predicted 4,578 open reading frames (Overbeek, R. et al. The SEED and the Rapid Annotation of microbial genomes using Subsystems Technology (RAST). Nucleic Acids Res. 42, D206-D214 (2013)). Of these, ˜63% reside on chromosome 1 and ˜37% reside on chromosome 2 (2,884 and 1,694 ORFs, respectively). Consistent with the broad metabolic capacity described for Vibrios, nearly half of all annotated ORFs are involved in carbohydrates, RNA and protein metabolism.

Several cellular processes have been implicated in rapid bacterial growth. Previous studies suggest bacteria can decrease generation time by initiating multiple rounds of genome replication. Alternatively, shorter generation time has been associated with increased capacity for protein biosynthesis, correlated with high copy numbers of rRNAs and tRNAs. While DNA replication and protein translation are intimately linked, it was nevertheless sought to tease apart their individual contributions to growth rate.

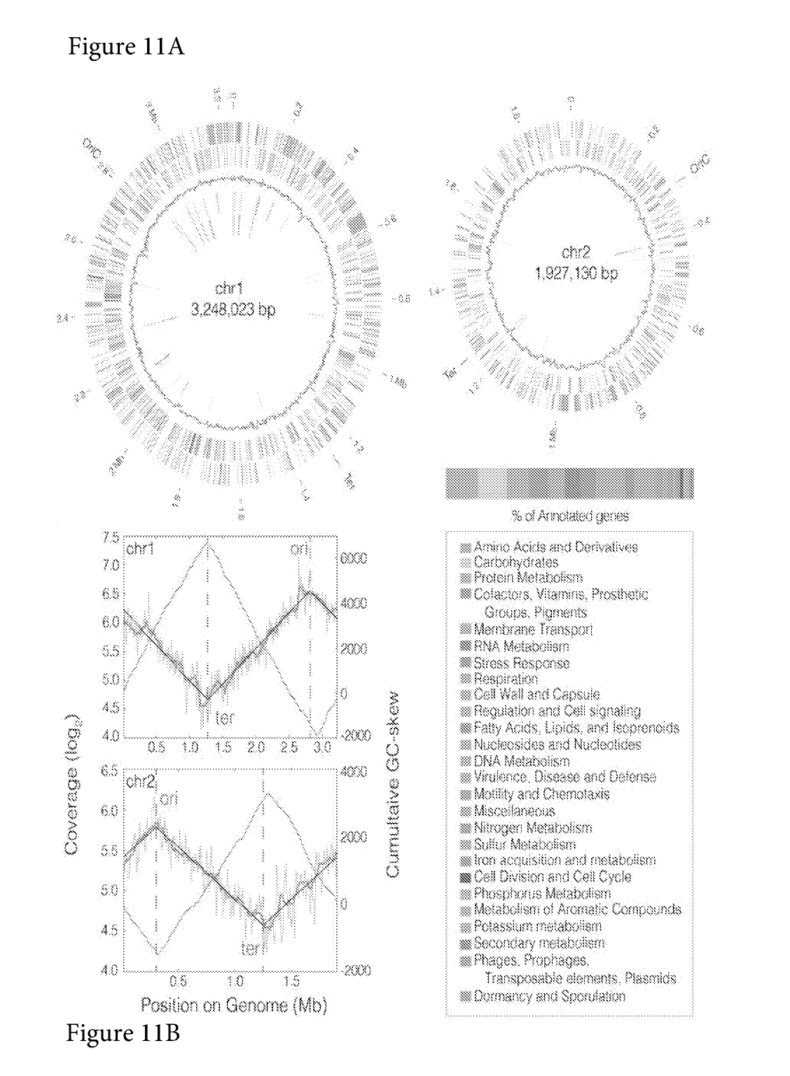

To examine the contribution of genome replication to rapid growth, we tested whether V. natriegens initiates more replication forks relative to E. coli. For this aim, we used sequencing to quantify genome coverage for both organisms in exponential and stationary growth phases. The peak-to-trough ratio (PTR), which represents sequencing coverage at the origin of replication (peak) relative to the terminus (trough), can be used as a quantitative measure of replication forks. Our results indicate the putative V. natriegens origin and terminus aligned with other Vibrios, and more replication forks are initiated on chr1 than chr2 (PTRs 3.67 and 2.4, respectively) (FIG. 11B). This result is consistent with observations in V. cholerae, where chr1 initiates earlier in cell cycle and sets the replication timing. However, the PTRs for E. coli and V. natriegens chr1 were nearly equivalent (PTRs 3.70 and 3.67, respectively), indicating similar number of replication forks. Thus, V. natriegens does not grow faster by initiating more replication forks.

Interestingly, an elevated number of tRNA and rRNAs in the V. natriegens genome was found. It contains 11 rRNA operons, compared with 7 and 8 operons in E. coli MG1655 and V. cholerae N16961, respectively (Table 1). Moreover, V. natriegens carries 129 tRNA genes, over 4-fold more than E. coli and V. cholerae. By transcriptional profiling of exponential growth under different temperatures (30° C., 37° C.) and media conditions (LB3, M9-glucose), we found that the most significant differentially expressed pathways by Gene Ontology (GO) are involved in ribosomal and protein biosynthesis (p-value <10⁻¹⁰).

To pinpoint genetic determinants for fast growth, high-throughput selections were devised to assess growth impact of all V. natriegens genes. As an initial approach, it was assessed whether V. natriegens genomic fragments could endow E. coli with enhanced generation time. However, such a mutant was unable to be isolated, suggesting that rapid growth is unlikely attributable to a single gene or copy number effects, particularly in light of unknown cross-species nuances. We also developed transposon systems and generated libraries of single-gene knockouts in V. natriegens, yet low insertion efficiency prevented scalable saturation mutagenesis. Instead, we turned to CRISPR/Cas9, which has found broad applicability in diverse hosts for targeted gene perturbation.

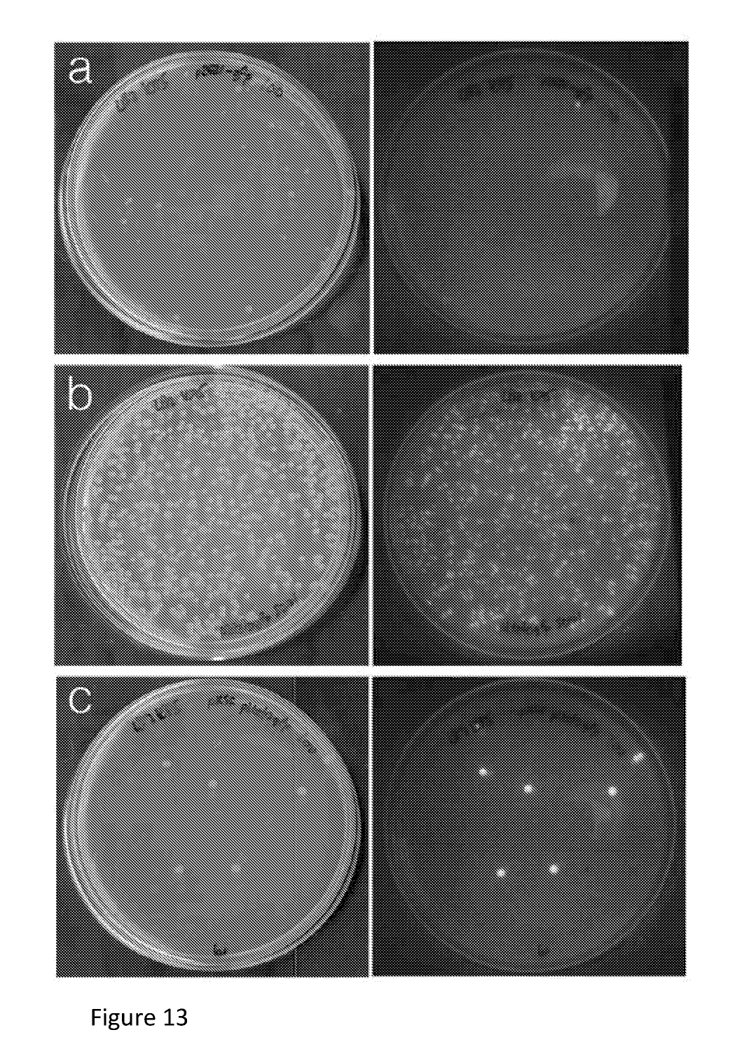

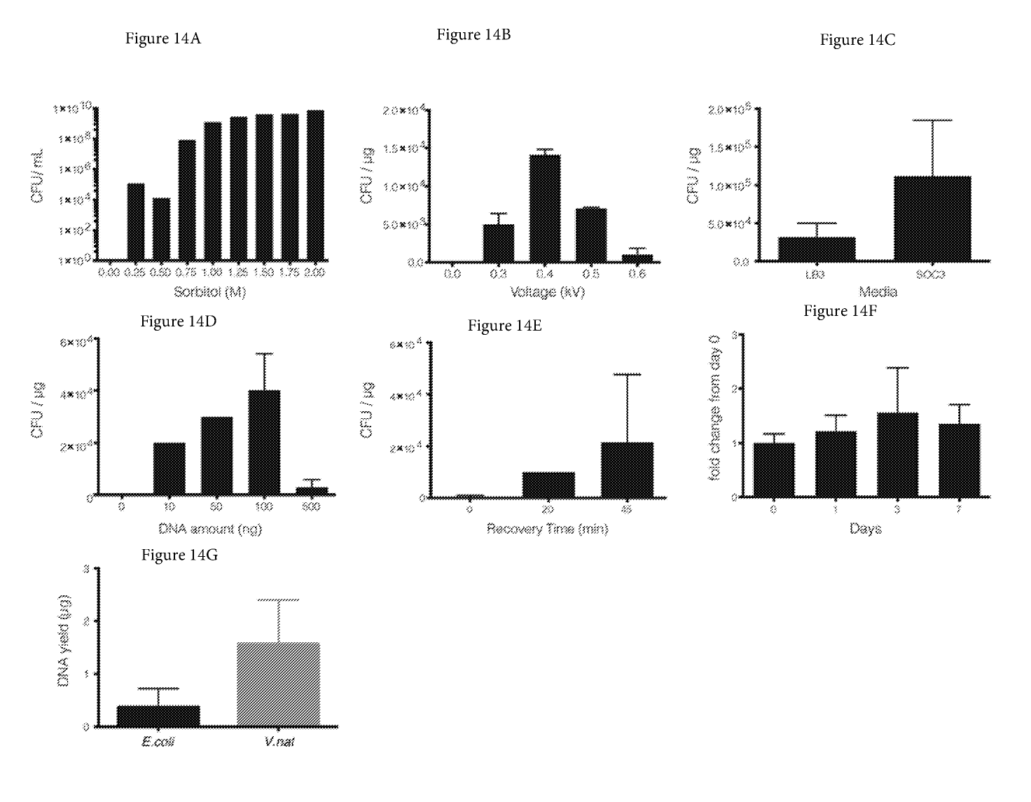

To facilitate genome-wide CRISPR/Cas9 screens, a high-efficiency transformation protocol was first established, achieving >2×10⁵ CFU/μg of plasmid DNA based on a broad-host range origin, RSF1010 (Katashkina, J. I. et al. Use of the λ Red-recombineering method for genetic engineering of Pantoea ananatis. BMC Mol. Biol. 10, 34 (2009)) (FIG. 13, FIG. 14). For modularity of Cas9 and guide RNA (gRNA) components, we engineered an additional shuttle vector based on the CTX vibriophage replicon (FIG. 15).

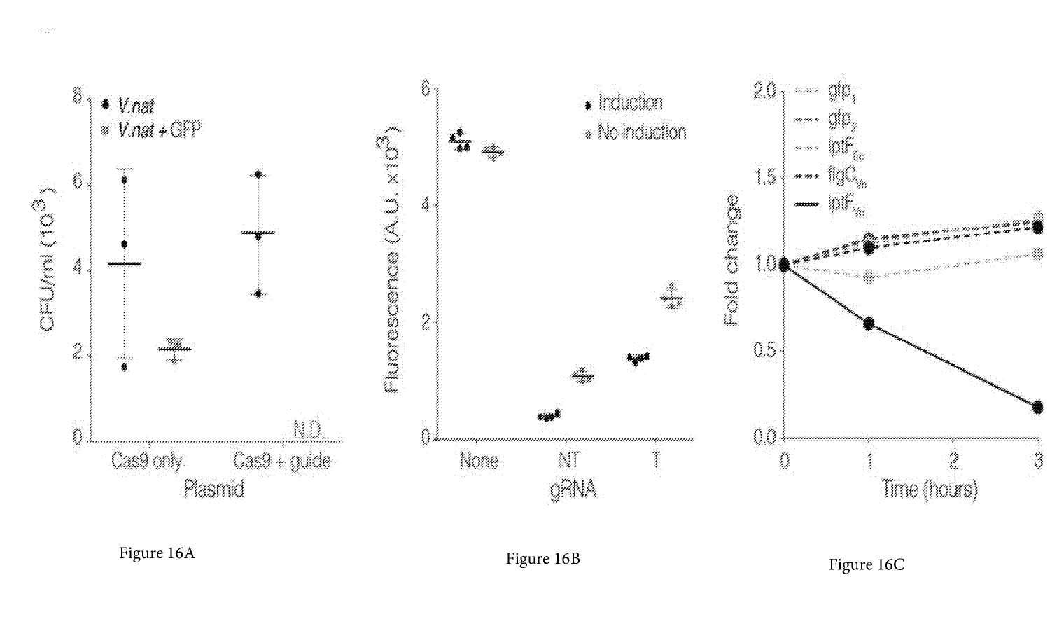

With these tools in hand, CRISPR/Cas9 functionality was established in V. natriegens. Consistent with observations in other bacteria, coexpression of a genome-targeting gRNA with Cas9 caused significant cellular toxicity. Furthermore, targeted inhibition of gene expression was demonstrated, using dCas9 a nuclease-deficient variant. Next, we prototyped a pooled CRISPRi assay. A small library of gRNAs was used, targeting putative growth neutral genes as well as a V. natriegens homolog of an essential E. coli gene. It was reasoned that if inhibition of a specific gene by a gRNA impairs cell growth, this gRNA would be depleted from the population under competitive growth conditions. Critically, it was found that gRNA abundance in a pooled CRISPRi screen could be used as a robust measure of a gene's impact on cellular fitness (FIG. 16). This scalable selection system enables rapid genome-wide profiling to identify genes responsible for cell fitness.

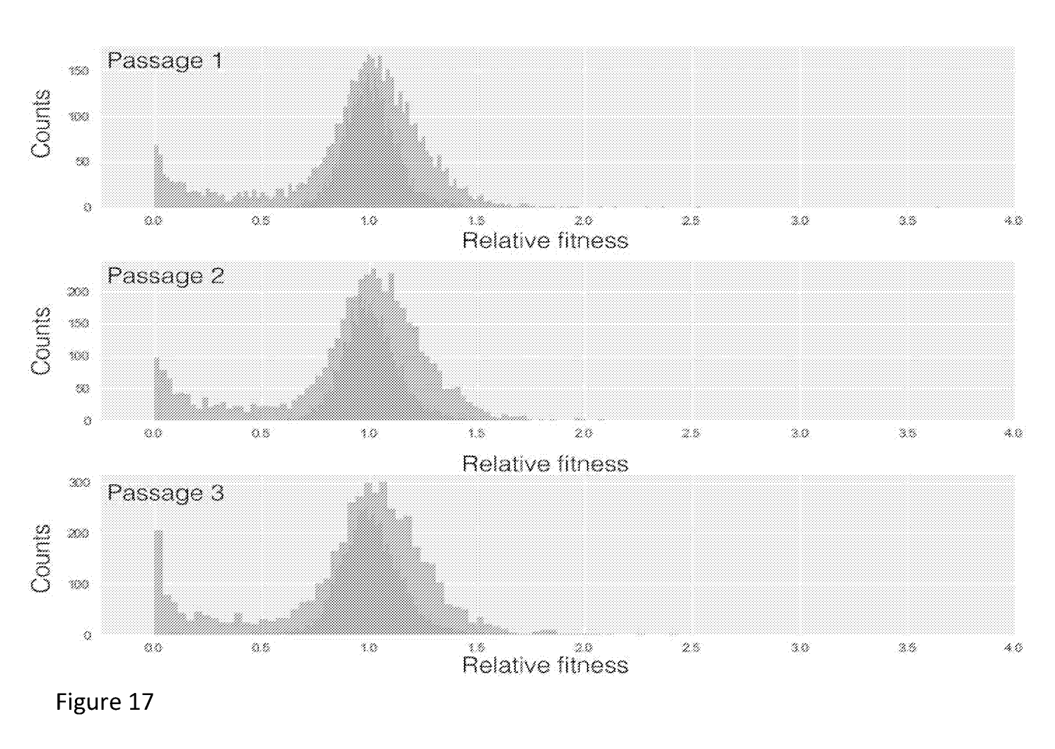

This assay was then used to comprehensively profile the relative fitness (RF) of 4,565 (99.7%) of RAST-predicted protein-coding V. natriegens genes under rapid growth conditions. We designed, assembled, and successfully transformed a library of 13,567 unique gRNAs into cells with or without dCas9 (FIG. 12A, FIG. 17). The library was grown in duplicate batch cultures to stationary phase, then serially passaged twice in fresh media to select for fast growing cells. We assigned relative fitness (RF) scores for each gene at each passage by computing the fold changes of its gRNAs' abundances at each time point relative to the initial condition.

Overall, 1070 genes were found to be essential for fast growth in V. natriegens. This set includes 604 putative essential genes, whose RF scores rapidly depleted in the first growth passage (RF≤0.529, p≤0.001, non-parametric), as well as 466 additional genes supporting fast growth whose RF were depleted throughout the three serial passages (RF≤0.781, p≤0.05, non-parametric) (FIG. 13B-C). Importantly, the majority of putative essential genes are in agreement with essentials in E. coli (250 of 354, 70%) and V. cholerae (289 of 449, 64.4%), identified by in-frame deletion or transposon mutagenesis. This degree of overlap is similar when comparing E. coli and V. cholerae alone (FIG. 12D). Furthermore, we found high agreement (52 of 59, 88%) of essentiality between V. natriegens ribosomal genes and their E. coli and V. cholerae homologs (FIG. 12E). The majority of essential genes (475 of 604, 78.6%) were assigned to RAST categories describing fundamental cell processes (FIG. 12F), with significant enrichment of GO categories for integral DNA, RNA, protein, and cellular energetic processes (p<0.05, BH-adjusted).

Analysis of the 466 subset sheds light on critical pathways for fast growth. RAST analysis indicated most genes are involved with amino acid (15.0%), carbohydrates (12.1%), and RNA metabolism (10%), with statistical enrichment of sulfur metabolism (RAST and GO:0070814, p<0.05, BH-adjusted) and RNA metabolism (RAST p<0.05, BH-adjusted). When considering all 1070 genes, GO categories related to protein translation were the most significantly enriched (p<10⁻¹⁰, BH-adjusted). A number of biological functions also became more significantly enriched relative to the essential set, the highest ranking being serine-family amino acids metabolism and tRNA modification (p<0.001, BH-adjusted). Interestingly, these processes include a number of tRNA synthetases, methylthiotransferases, and threonylcarbamoyl adenosine (t6A) modification enzymes.

The co-enrichment of assimilatory sulfur pathway enzymes and multiple translation-related categories points to a key role for sulfur-based translational regulation in rapid V. natriegens growth. Specifically, post-transcriptional tRNA modifications, such as sulfur-dependent tRNA thiolation enzymes which are enriched in this gene set, are critical checkpoints for regulating tRNA integrity and translation rate (Laxman, S. et al. Sulfur amino acids regulate translational capacity and metabolic homeostasis through modulation of tRNA thiolation. Cell 154, 416-429 (2013), Nakai, Y., Nakai, M. & Hayashi, H. Thio-modification of yeast cytosolic tRNA requires a ubiquitin-related system that resembles bacterial sulfur transfer systems. J. Biol. Chem. 283, 27469-27476 (2008)) and are synchronized with bacterial growth rate (Emilsson, V., Naslund, A. K. & Kurland, C. G. Thiolation of transfer RNA in Escherichia coli varies with growth rate. Nucleic Acids Res. 20, 4499-4505 (1992)). Furthermore, the universal tRNA modification t6A, essential in many bacteria (El Yacoubi, B., Bailly, M. & de Crécy-Lagard, V. Biosynthesis and function of posttranscriptional modifications of transfer RNAs. Annu. Rev. Genet. 46, 69-95 (2012)), has been shown to affect the speed of tRNA charging and translation fidelity in vitro and its depletion in vivo results in pleiotropic and negative consequences for cell growth (Thiaville, P. C. et al. Essentiality of threonylcarbamoyladenosine (t6A), a universal tRNA modification, in bacteria. Mol. Microbiol. 98, 1199-1221 (2015), Thiaville, P. C. et al. Global translational impacts of the loss of the tRNA modification t(6)A in yeast. Microb. Cell Fact. 3, 29-45 (2016)).

Several genes resulted in increased RF scores upon dCas9 inhibition, which could indicate either improved growth under these conditions or limitations of this experimental system. These include DNA helicase recQ, periplasmic transporter potD, Na+/H+ antiporter NhaP, biotin synthesis protein bioC, and Glutamate-aspartate transporter gltJ. Further work is required to assess the biological relevance of gene perturbations resulting in enhanced RF scores. It is important to note that genes affecting CRISPRi regulation or plasmid replication may bias this assay. Additional studies are warranted to assess these scores with alternative genetic methods and diverse experimental conditions as well as to map higher-order genetic interactions.

The gene sets defined in this study will serve as a basis for advanced studies and engineering of V. natriegens. For example, these RF scores could inform bottom-up construction and validation of fast growing synthetic bacteria. Furthermore, these gene sets will be useful for probing the limits of codon reassignment in V. natriegens (Ostrov, N. et al. Design, synthesis, and testing toward a 57-codon genome. Science 353, 819-822 (2016), Lee, H. H., Ostrov, N., Gold, M. A. & Church, G. M. Recombineering in Vibrio natriegens. bioRxiv 130088 (2017). doi:10.1101/130088). The spatial distribution of these genes across the two chromosomes also presents fascinating opportunities for rational genome design. Intriguingly, only 4.3% (26 of 604) of essential genes and 11.7% (125 of 1070) of fast growth genes are located on chr2 (FIG. 13C). Consolidation of functional genes to chr1 could allow repurposing of chr2 origin as an artificial chromosome for stable replication of large pieces of heterologous DNA.

Forward Genetic Screen in E. coli to Identify V. natriegens Genes for Fast Growth.

We performed gain-of-function growth screens in E. coli, to explore whether its growth rate could be enhanced by expression of V. natriegens genes. To de-risk this strategy, we first sought to assess whether V. natriegens homolog genes could functionally rescue E. coli mutants. We opted for an antibiotic challenge assay using recA, a widely conserved DNA repair protein. RecA deleted mutants are sensitive to a wide range of antibiotics, including the quinolones antibiotic ciprofloxacin which induces double-stranded DNA breaks and SOS DNA damage repair response.

We cloned V. natriegens recA (recAvₙ, FIG|691.12.PEG.183) under the control of the constitutive promoter pLtetO, and introduced the plasmid in trans to E. coli ArecA strain (Baba, T. et al. Construction of Escherichia coli K-12 in-frame, single-gene knockout mutants: the Keio collection. Mol. Syst. Biol. 2, 2006.0008 (2006), Lutz, R. & Bujard, H. Independent and tight regulation of transcriptional units in Escherichia coli via the LacR/O, the TetR/O and AraC/I1-I2 regulatory elements. Nucleic Acids Res. 25, 1203-1210 (1997)). We then assayed colony survival with two concentrations of ciprofloxacin. Wild-type E. coli and ΔrecA mutant lacking recA_{Vn} were used as controls. No colonies were observed on antibiotic-containing plates using the mutant ΔrecA control strain. For wild type E. coli, we found no colonies using 25 ng/mL ciprofloxacin and mild defects in colony formation using 10 ng/mL ciprofloxacin. In contrast, the mutant strain carrying recA_{Vn} showed rescue of E. coli colony growth at both ciprofloxacin concentrations. These data indicate that V. natriegens genes can be functional in E. coli.

We next sought to increase the diversity and scale of this screen. We generated a fosmid library carrying large (>24 kb) fragments of genomic DNA from either E. coli MG1655, a control to assess whether increase in copy number of endogenous genes would itself be advantageous to growth, or V. natriegens. After daily serial passaging in glucose-supplemented M9 media, we sequenced the fosmid library and found that V. natriegens sequences were depleted in the population. For example, by day 2 of our experiment 99.9% of all sequences were from E. coli while only 0.1% were from V. natriegens. Specifically, the recovered E. coli genome sequences encoded the arabinose utilization operon and the valine, leucine, and isoleucine biosynthesis operon, two operons which were deficient in the host E. coli EPI300-T1^{R} (Epicentre) and were highly selected for in our screen. Critically, we did not find enrichment of the homologous V. natriegens arabinose genes. We repeated this screen in E. coli cells carrying T7 polymerase (T7 Express, NEB) but could not detect any faster growing variants.

Taken together, we conclude that E. coli growth speed could not be accelerated by increases in gene dosage of endogenous genes, even at 10-fold abundance, or by introduction of any contiguous V. natriegens genome fragment. The genetic determinants for rapid growth are unlikely to be directly portable from V. natriegens to other species by transfer of a single contiguous genomic fragment.

### Plasmid Stability and Yield

Initial transformations with E. coli plasmids carrying constitutively-expressing GFP yielded variability in colony size and fluorescence, suggestive of plasmid instability (Hamashima, H., Iwasaki, M. & Arai, T. A Simple and Rapid Method for Transformation of Vibrio Species by Electroporation. in Electroporation Protocols for Microorganisms (ed. Nickoloff, J. A.) 155-160 (Humana Press)). Instead, we found that a plasmid based on the broad-host range RSF1010 origin yielded transformants more consistent in morphology and fluorescence (Katashkina, J. I. et al. Use of the λ Red-recombineering method for genetic engineering of Pantoea ananatis. BMC Mol. Biol. 10, 34 (2009)) (FIG. 13). We developed a high efficiency method for introduction of recombinant DNA into V. natriegens. This protocol can generate electrocompetent cells in 2 hours which are also suitable for direct long-term storage at −80° C. Transformation efficiencies up to 2×10⁵ CFU/μg can be achieved and transformants can be obtained with as little as 10 ng plasmid DNA. Transformants can be visualized and picked after 5 hours of plating. Furthermore, 2 μg of plasmid DNA can be isolated within 5 hours of growth, ˜2.5× more than equivalent E. coli culture (FIG. 14).

Harnessing the CTX Replicon as a New V. natriegens Plasmid

In search of additional stable replicons, we turned to bacteriophages. Like the coliphage M13, whose replicative form (RF) served as a basis for early E. coli plasmids, we used the CTX vibriophage (Waldor, M. K. & Mekalanos, J. J. Lysogenic conversion by a filamentous phage encoding cholera toxin. Science 272, 1910-1914 (1996)). Transformation of CTX RF in V. natriegens yielded robust transformants. We thus constructed a new shuttle plasmid, pRST, by fusing CTX replication genes with the conditionally replicating R6K origin for cloning in E. coli.

We further tested the infectivity of CTX on V. natriegens. Importantly, we found that the CTX bacteriophage was >100-fold less infective of V. natriegens compared to V. cholerae 0395. Furthermore, we could not detect production of infective viral CTX particles in the supernatant of V. natriegens transformants which had undergone direct electroporation of CTX replicative form, showing that CTX viral particles are either not produced or not functionally assembled in V. natriegens. Given the low rates of infectivity and the fact that CTX virions are not found in high-titers in the environment, we conclude V. natriegens is an unlikely host for the propagation of CTX phage (Davis, B. Filamentous phages linked to virulence of Vibrio cholerae. Curr. Opin. Microbiol. 6, 35-42 (2003)). These tests further support the Biosafety Level 1 (BSL-1) designation for V. natriegens as generally safe biological agent.

### Transposon Mutagenesis Saturation and Characterization

We observed low saturation of transposon mutagenesis in V. natriegens, with only 47.7% of genes containing one insertion and 23.4% containing ≥2 insertions. Further analysis revealed that a large percentage of the transposon library sequencing reads mapped to the transposon backbone. This is indicative of genomic integration of the suicide transposon vector since no plasmids could be extracted from V. natriegens transconjugants, and direct electroporation of the transposon plasmid alone into V. natriegens did not produce any detectable transformants. Interestingly, genomic integration of a suicide transposon vector following conjugation in V. cholerae strains has been observed, and the underlying mechanisms of this activity is not well understood. Deeper investigation into these integration events may improve the fidelity of transposon mutagenesis in V. natriegens.

We also analyzed transposon mutant colonies in greater detail by whole genome sequencing. When grown without antibiotic selection for the transposon, we were unable to find sequencing reads that mapped to the transposon, indicating instability and excision of this genomic element. Additionally, we found that some mutants carried genomic sequences which mapped to portions or all of the transposon suicide vector, including the Himarl transposase, the ampicillin marker, and the oriT and R6K origin. These excisions and integrations greatly impede high-throughput identification of insertion locations since no common sequence can be used to determine the junction between integrated DNA and genomic DNA. Deep sequencing of specific mutants can, however, enable identification of the genetic perturbation underlying a specific phenotype.

Growth Rate of V. natriegens Strain Having recQ Gene Suppressed

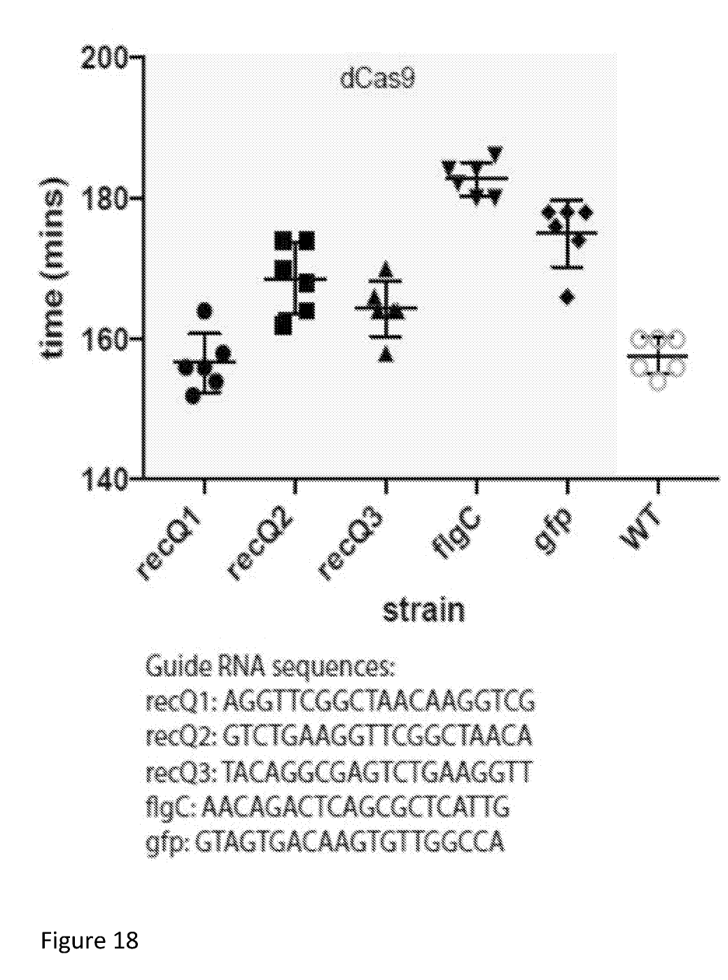

We took the gene with the highest increase in fold change from our pooled screen, recQ, and assayed its growth rate individually. We used 3 different guide RNAs targeting various regions of the gene and found that the resulting mutant grows significantly faster (p<0.01) than those where guide RNAs were used to target a growth-neutral gene (flgC) or an off-target gene (gfp, which doesn't exist in the genome). It was found that inhibiting recQ using the recQ1 guideRNA gave rise to cells that grew slightly faster than the wild-type strains, which were not burdened by dCas9 nor guide RNA. (FIG. 18) Taken together, the data suggested that if a recQ mutant was generated based our recombineering strategy, the mutant strain should grow faster than wild-type.

### Materials and Methods

### Data Availability

Genome sequences are available in NCBI (GenBank CP009977-8, RefSeq NZ_CP009977-8). Transcriptome data will be made available in NCBI GEO. All other data are available in the Supplementary Information, or by request.

### Growth Media

Unless denoted, LB3, Lysogeny Broth with 3% (w/v) final NaCl, is used as standard rich media. We prepare this media by adding 20 grams of NaCl to 25 grams of LB Broth-Miller (Fisher BP9723-500). Media are formulated according to manufacturer instructions and supplemented with 1.5% (w/v) final Ocean Salts (Aquarium System, Inc.) to make high salt versions of Brain Heart Infusion (BHIO), Nutrient Broth (NBO), and Lysogeny Broth (LBO). No additional salts were added to Marine Broth (MB). Minimal M9 media was prepared according to manufacturer instruction. For culturing V. natriegens, 2% (w/v) final NaCl was added to M9. Carbon sources were added as indicated to 0.4% (v/v) final. SOC3 media is composed of 5 grams of yeast extract, 20 grams tryptone, 30 grams sodium chloride, 2.4 grams magnesium sulfate, and 0.4% (w/v) final glucose. Antibiotic concentrations used for plasmid selection in V. natriegens: ampicillin/carbenicillin 100 μg/ml, kanamycin 75 μg/ml, chloramphenicol 5 μg/ml, spectinomycin 100 μg/ml. E. coli experiments were performed in standard LB media and M9.

### Overnight Culturing

An inoculation of −80° C. frozen stock of V. natriegens can reach stationary phase after 5 hours when incubated at 37° C. Prolonged overnight culturing (>15 hours) at 37° C. may lead to an extended lag phase upon subculturing. Routine overnight culturing of V. natriegens was performed for 8-15 hours at 37° C. or 12-24 hours at room temperature. Unless otherwise indicated, E. coli cells used in this study were K-12 subtype MG1655 cultured overnight (>10 hours) at 37° C. V. cholerae 0395 was cultured overnight (>10 hours) in LB at 30° C. or 37° C. in a rotator drum at 150 rpm.

### Glycerol Stock

To prepare V. natriegens cells for −80° C. storage, an overnight culture of V. natriegens is washed in fresh media before storing in glycerol. Cultures were centrifuged for 1 minute at 20,000 rcf and the supernatant was removed. The cell pellet was resuspended in fresh LB3 media and glycerol was added to 20% final concentration. The stock is quickly vortexed and stored at −80° C. Bacterial glycerol stocks stored in this manner are viable for at least 5 years.

### Bulk Measurements of Generation Time

Growth was measured by kinetic growth monitoring (Biotek H1, H4, or Eon plate reader) in 96-well plates with continuous orbital shaking and optical density (OD) measurement at 600 nm taken every 2 minutes. Overnight cells were washed once in fresh growth media, then subcultured by at least 1:100 dilution. To assay V. natriegens growth in different rich media, cells were cultured overnight from frozen stock into the respective media. To assay V. natriegens and E. coli growth in minimal media, cells were cultured overnight in LB3 and LB respectively, and subcultured in the appropriate test media. Generation times were calculated by linear regression of the log-transformed OD across at least 3 data points when growth was in exponential phase. To avoid specious determination of growth rates due to measurement noise, the minimal OD considered for analysis was maximized and the ODs were smoothed with a moving average window of 3 data points for conditions that were challenging for growth.

### Microfluidics Device Construction

Microfluidic devices were used as tools to measure and compare growth rates of E. coli and V. natriegens. In these devices, cells are grown in monolayer and segmented/tracked in high temporal resolution using time-lapse microscopy. The cells are constricted for imaging using previously described Tesla microchemostat device designs, in which cell traps have heights that match the diameters of the cells, minimizing movement and restricting growth in a monolayer (Cookson, S., Ostroff, N., Pang, W. L., Volfson, D. & Hasty, J. Monitoring dynamics of single-cell gene expression over multiple cell cycles. Mol. Syst. Biol. 1, 2005.0024 (2005), Stricker, J. et al. A fast, robust and tunable synthetic gene oscillator. Nature 456, 516-519 (2008), Vega, N. M., Allison, K. R., Khalil, A. S. & Collins, J. J. Signaling-mediated bacterial persister formation. Nat. Chem. Biol. 8, 431-433 (2012)). Different trapping heights of 0.8 μm and 1.1 μm were used for E. coli and V. natriegens, respectively. Microfluidic devices were fabricated with polydimethylsiloxane (PDMS/Sylgard 184, Dow Corning) using standard soft lithographic methods¹⁰. Briefly, microfluidic devices were fabricated by reverse molding from a silicon wafer patterned with two layers of photoresist (one for the cell trap, another for flow channels). First, the cell trap layer was fabricated by spin coating SU-8 2 (MicroChem Corp.) negative resist at 7000 RPM and 6800 RPM for E. coli and V. natriegens, respectively, and patterned using a high resolution photomask (CAD/Art Services, Inc.). Next, AZ4620 positive photoresist (Capitol Scientific, Inc.) was spun onto the silicon wafer and aligned with another photomask for fabrication of ˜8 μm tall flow channels (same for both organisms). Reverse-molded PDMS devices were punched and bonded to No. 1.5 glass coverslips (Fisher Scientific), similar to previously described protocols (Duffy, D. C., McDonald, J. C., Schueller, O. J. & Whitesides, G. M. Rapid Prototyping of Microfluidic Systems in Poly(dimethylsiloxane). Anal. Chem. 70, 4974-4984 (1998)).

### Time-Lapse Microscopy and Image Analysis

Cells were diluted down to 0.1 OD₆₀₀ from an overnight culture at optimal growth conditions and allowed to grow for an hour in the corresponding media conditions (e.g. temperature, salt concentration) before loading onto the device. Next, cells were loaded and grown on the device in the corresponding environmental conditions until the cell trap chambers filled. Temperature was maintained with a Controlled Environment Microscope Incubator (Nikon Instruments, Inc.). Media flow on device was maintained by a constant pressure of 5 psi over the course of the experiment after cell loading. During the experiment, phase contrast images were acquired every minute with a 100× objective (Plan Apo Lambda 100X, NA 1.45) using an Eclipse Ti-E inverted microscope (Nikon Instruments, Inc.), equipped with the “Perfect Focus” system, a motorized stage, and a Clara-E charge-coupled device (CCD) camera (Andor Technology). After the experiment, individual cells were segmented from the image time course using custom MATLAB (Mathworks, Natick, Mass.) software. Doubling time of cells was scored well before the density of the chamber impacted tracking and growth of cells. Results from repeat experiments on different days and devices were consistent (Data not shown).

### Genome Sequencing by Pacific Bioscience Sequencing, De Novo Assembly, and Annotation

V. natriegens (ATCC 14048) was cultured for 24 hours at 30° C. in Nutrient Broth with 1.5% NaCl according to ATCC instructions. Genomic DNA was purified (Qiagen Puregene Yeast/Bact. Kit B) and sequenced on a Single Molecule Real Time (SMRT) Pacific Biosciences RS II system (University of Massachusetts Medical School Deep Sequencing Core) using 120 minute movies on 3 SMRTCells. SMRTanalysis v2.1 on Amazon Web Services was used to process and assemble the sequencing data. The mean read length, after default quality filtering, was 4,407 bp. HGAP3 with default parameters was used to assemble the reads which yielded 2 contigs. The contigs were visualized with Gepard and manually closed (Krumsiek, J., Arnold, R. & Rattei, T. Gepard: a rapid and sensitive tool for creating dotplots on genome scale. Bioinformatics 23, 1026-1028 (2007)). The two closed chromosomes annotated using RAST under ID 691.12 (Aziz, R. K. et al. The RAST Server: rapid annotations using subsystems technology. BMC Genomics 9, 75 (2008)). The annotated genome is deposited in NCBI under Biosample SAMN03178087, GenBank CP009977-8, RefSeq NZ_CP009977-8. Base modification detection was performed on SMRTanalysis v2.1 with default setting and the closed genome as reference. Codon usage was calculated using EMBOSS cusp.

### Quantifying Genome Replication Forks by Oxford Nanopore Sequencing

V. natriegens was cultured in LB3 and E. coli was cultured in LB. Both cultures were grown overnight at 37° C. For stationary phase samples, 1 mL of each culture was collected for genomic DNA extraction. For exponential phase samples, each culture was subcultured and grown to OD₆₀₀ ˜0.4 and 10 mL of each was collected for genomic DNA extraction. Genomic DNA was purified (Qiagen Puregene Yeast/Bact. Kit B). To maximize read length, ˜1 μg of genomic DNA for each sample was used as input. 1D sequencing libraries were prepared, barcoded (SQK-RAD002 and SQK-RBK001), and sequenced on the MinION with SpotON R9.4 flow cells for 48 hours. Cloud base-calling and sample demultiplexing was performed on Metrichor 1.4.5 and FASTQ files prepared from FASTS HDF files with a custom python script. Sequences were aligned to the reference genome using GraphMap 0.5.1 (Sovic, I. et al. Fast and sensitive mapping of nanopore sequencing reads with GraphMap. Nat. Commun. 7, 11307 (2016)). Coverage was computed with bedtools 2.26.0 and PTR computed using the iRep package (Quinlan, A. R. & Hall, I. M. BEDTools: a flexible suite of utilities for comparing genomic features. Bioinformatics 26, 841-842 (2010), Brown, C. T., Olm, M. R., Thomas, B. C. & Banfield, J. F. Measurement of bacterial replication rates in microbial communities. Nat. Biotechnol. 34, 1256-1263 (2016)).

### Transcriptome Profiling

Triplicate V. natriegens cultures were grown overnight from −80° C. stocks for each condition to be assayed: 30° C. in LB3, 37° C. in LB3 and 37° C. in M9 high-salt media supplemented with 2% (w/v) final sodium chloride and 0.4% (w/v) glucose. Each culture was subcultured in the desired conditions and grown to exponential phase (OD₆₀₀ 0.3-0.6). To collect RNA, 10 mL of each culture was stabilized with Qiagen RNAprotect Bacteria Reagent and frozen at −80° C. RNA extraction was performed with Qiagen RNeasy Mini Kit and rRNA depleted with Illumina Ribo-Zero rRNA Removal Kit (Bacteria). Samples were spot-checked for RNA sample quality on an Agilent 2100 RNA 6000 Nano Kit to ensure that the RNA Integrity Number (RIN) was above 9. Sequencing libraries were prepared with the NEXTflex Rapid Directional qRNA-Seq Kit. Each sample was barcoded and amplified with cycle-limited real-time PCR with KAPA SYBR FAST. Resulting libraries were sequenced with MiSeq v3 150 to obtain paired end reads.

Sequences were trimmed with cutadapt (Martin, M. Cutadapt removes adapter sequences from high-throughput sequencing reads. EMBnet. journal 17, 10-12 (2011)). Transcripts were quantified with Salmon 0.8.1 and counts were summarized with tximport for differential expression analysis with DESeq2 (Patro, R., Duggal, G., Love, M. I., Irizarry, R. A. & Kingsford, C. Salmon provides fast and bias-aware quantification of transcript expression. Nat. Methods 14, 417-419 (2017), Love, M. I., Huber, W. & Anders, S. Moderated estimation of fold change and dispersion for RNA-seq data with DESeq2. Genome Biol. 15, 550 (2014), Soneson, C., Love, M. I. & Robinson, M. D. Differential analyses for RNA-seq: transcript-level estimates improve gene-level inferences. F1000Res. 4, 1521 (2015)). Gene Ontology annotations were extracted by mapping V. natriegens genes with eggnog-mapper based on eggNOG 4.5 orthology data (Huerta-Cepas, J. et al. Fast genome-wide functional annotation through orthology assignment by eggNOG-mapper. Mol. Biol. Evol. (2017). doi:10.1093/molbev/msx148, Huerta-Cepas, J. et al. eggNOG 4.5: a hierarchical orthology framework with improved functional annotations for eukaryotic, prokaryotic and viral sequences. Nucleic Acids Res. 44, D286-93 (2016), Yamazaki, Y., Niki, H. & Kato, J.-I. Profiling of Escherichia coli Chromosome database. Methods Mol. Biol. 416, 385-389 (2008), Chao, M. C. et al. High-resolution definition of the Vibrio cholerae essential gene set with hidden Markov model-based analyses of transposon-insertion sequencing data. Nucleic Acids Res. 41, 9033-9048 (2013)). Functional enrichment computed with AmiGO in Cytoscape (Carbon, S. et al. AmiGO: online access to ontology and annotation data. Bioinformatics 25, 288-289 (2009), Shannon, P. et al. Cytoscape: a software environment for integrated models of biomolecular interaction networks. Genome Res. 13, 2498-2504 (2003)).

Fosmid-Based Gain-of-Function Screen in E. coli

Input genomic DNA was prepared (Epicentre MasterPure DNA) and pulse-field electrophoresis verified that the major band of isolated DNA was ˜50 kb. The fosmid library was prepared and packaged with T1 phage (Epicentre CopyControl Fosmid Library Kit). To verify the insert size, fosmids were extracted (Epicentre Fosmid Extraction Kit), restricted with NotI to release the insert and pulse-field electrophoresis verified that resulting inserts were 24-48 kb. We then transduced E. coli EPI300-T1^{R} with packaged phages carrying either E. coli MG1655 or V. natriegens genomic DNA. We collected ˜160,000 colonies for each sample type, ensuring >99% probability of representation of the entire E. coli or V. natriegens genome (Sambrook, J., Fritsch, E. F., Maniatis, T. & Others. Molecular cloning: a laboratory manual. (Cold spring harbor laboratory press, 1989)). This pool represents our shotgun growth library. We further verified high coverage of both E. coli and V. natriegens genomes by Illumina sequencing. We observe an average of 74× coverage for E. coli and 109× and 281× coverage for chromosome 1 and 2 for V. natriegens, respectively. For initial growth screen, we serially passaged our shotgun pool for 7 days in M9+0.4% (w/v) final glucose and 1× CopyControl Induction Solution to increase fosmid copy number. We started with an initial 50:50 mixture of EPI300-T1^{R} carrying genomic pieces of E. coli MG1655 or V. natriegens in M9+0.4% (w/v) final glucose. Every 24 hours, the library was diluted 1:1000 into the same media composition as the start. Fosmids of this library mixture was isolated at days 0, 1, 2, 4, and 7. These samples were sequenced on a MiSeq as paired end 30 bp reads and sequences mapped to their respective reference genomes with Bowtie (Langmead, B., Trapnell, C., Pop, M. & Salzberg, S. L. Ultrafast and memory-efficient alignment of short DNA sequences to the human genome. Genome Biol. 10, R25 (2009)). Our sequencing reads verified high coverage of our initial starting fosmid libraries for both E. coli (74× coverage) and V. natriegens (chr1: 109×, chr2: 281× coverage). Gain-of-function screen with T7 expression (T7 Express, NEB) were cultured similarly except in LB.

### Construction of Transposon Mutant Libraries

To facilitate transposon mutagenesis, we engineered a suicide mariner-based transposon vector modified for insertion mapping by high-throughput sequencing (van Opijnen, T. & Camilli, A. Genome-wide fitness and genetic interactions determined by Tn-seq, a high-throughput massively parallel sequencing method for microorganisms. Curr. Protoc. Microbiol. Chapter 1, UnitlE.3 (2010), Cameron, D. E., Urbach, J. M. & Mekalanos, J. J. A defined transposon mutant library and its use in identifying motility genes in Vibrio cholerae. Proc. Natl. Acad. Sci. U.S.A. 105, 8736-8741 (2008), Martínez-García, E., Calles, B., Arévalo-Rodríguez, M. & de Lorenzo, V. pBAM1: an all-synthetic genetic tool for analysis and construction of complex bacterial phenotypes. BMC Microbiol. 11, 38 (2011)). Our conjugative suicide mariner transposon plasmid was propagated in BW29427, an E. coli with diaminopimelic acid (DAP) auxotrophy. BW29427 growth requires 300 μM of DAP even when cultured in LB. Importantly, BW29427 does not grow in the absence of DAP, which simplifies counterselection of this host strain following biparental mating with V. natriegens. For conjugation from E. coli to V. natriegens, 24 mL of each strain was grown to OD 0.4, spun down, resuspended and plated on LB2 plates (Lysogeny Broth with 2% (w/v) final of sodium chloride) and incubated at 37° C. for 60 minutes. This conjugation time was chosen to minimize clonal amplification, based on optimization experiments using 100 uL of each strain. The cells are recovered from the plate in 1 mL of LB3 media. The resulting cell resuspension is washed once in fresh LB3, resuspended to a final volume of 1 mL, and plated on 245 mm×245 mm kanamycin selective plates (Corning). Plates were incubated at 30° C. for 12 hours to allow the formation of V. natriegens colonies. Colonies were scraped from each plate with 3 mL of LB3, gently vortexed, and stored as glycerol stock as previously described. No colonies were detected in control experiments with only BW29427 donor cells. A similar protocol was used to generate an E. coli transposon mutant library, except LB was used as the media at all steps.

### Analysis of the Transposon Mutant Library

Briefly, genomic DNA was extracted (Qiagen DNeasy Blood & Tissue Kit), and digested with MmeI. To enrich for the fragment corresponding to the kanamycin transposon fragment, the digested genomic DNA was electrophoresed on a 1% TAE gel and an area of the gel corresponding to approximately 1.2 kb was extracted. The resulting DNA fragment was sticky-end ligated to an adapter. PCR was used to selectively amplify the region around the transposon mosaic end and to add the required Illumina adapters. These amplicons were sequenced 1×50 bp on a MiSeq. Since properly prepared amplicons contain 16 or 17 bp of genomic DNA and 32 or 33 bp of the ligated adapter, only those sequencing reads with the presence of the adapter were further analyzed. All adapters were trimming and the resulting genomic DNA sequences were aligned to the reference genome with Bowtie²⁷. Statistical enrichment of RAST categories were computed with the hypergeometric test and resulting p-values were adjusted with Benjamini-Hochberg correction (Benjamini, Y. & Hochberg, Y. Controlling the False Discovery Rate: A Practical and Powerful Approach to Multiple Testing. J. R. Stat. Soc. Series B Stat. Methodol. 57, 289-300 (1995)).

For the E. coli Himarlmutant library, we isolated 1.1×10⁶ transconjugants, prepared Tn-Seq fragments as previously described, and analyzed by MiSeq (van Opijnen, T. & Camilli, A. Genome-wide fitness and genetic interactions determined by Tn-seq, a high-throughput massively parallel sequencing method for microorganisms. Curr. Protoc. Microbiol. Chapter 1, Unit1E.3 (2010)). We obtained 6.9×10⁶ total reads, of which 1.6% mapped to the transposon plasmid; 98.3% of filtered reads were mapped to the genome. These insertions represent 107,723 unique positions, where >10 unique insertions were present in 3,169 out of 4,917 features. For the V. natriegens Himarl mutant library we isolated 8.6×10⁵ mutants. We obtained 6.1×10⁶ reads, of which 36.4% mapped to the transposon plasmid; 97.2% of filtered reads were mapped to the genome. These insertions represent 4,530 unique positions, proportionally distributed between the two chromosomes where >1 unique insertions were found in 2,357 out of 4,940 features.

Isolation of Motility Phenotypes from Transposon Library

Single transposon library colonies were isolated on 1.5% agar plates and grown to density overnight at 30° C. or 37° C. in liquid LB3 media. 1 μl of overnight culture was applied at the center of LB3+0.3% agar plates (LB+0.3% agar for E. coli and V. cholerae) and incubated at the indicated temperature. Plates were scanned using Epson Expression 10000 XL desktop scanner and colony radius, in pixels, was measured using ImageJ.

Electroporation Protocol for DNA Transformation of V. natriegens

An overnight V. natriegens culture was pelleted, washed once in fresh media, and diluted 1:100 into growth media. Cells were harvested at OD₆₀₀-0.4 (1 hour growth when incubated at 37° C. at 225 rpm) and pelleted by centrifugation at 3500 rpm for 5 min at 4° C. The pellet is washed three times using 1 ml of cold 1M sorbitol and centrifuged at 20,000 rcf for 1 minute at 4° C. The final cell pellet was resuspended in 1M sorbitol at a 200-fold concentrate of the initial culture. For long term storage, the concentrated competent cells were aliquoted in 50 μL shots in pre-chilled tubes, snap frozen in dry ice and ethanol, and stored in −80° C. for future use. To transform, 50 ng of plasmid DNA was added to 50 μL of concentrated cells in 0.1 mm cuvettes and electroporated using Bio-Rad Gene Pulser electroporator at 0.4 kV, 1 kΩ, 25 μF and recovered in 1 mL LB3 or SOC3 media for 45 minutes at 37° C. at 225 rpm, and plated on selective media. Plates were incubated at least 6 hours at 37° C. or at least 12 hours at room temperature.

### Plasmid Construction

Routine cloning was performed by PCR of desired DNA fragments, assembly with NEB Gibson Assembly or NEBuilder HiFi DNA Assembly, and propagation in E. coli unless otherwise indicated (Gibson, D. & Daniel, G. One-step enzymatic assembly of DNA molecules up to several hundred kilobases in size. Protocol Exchange (2009). doi: 10.1038/nprot.2009.77). We used pRSF for the majority of our work since it carries all of its own replication machinery and should be minimally dependent on host factors (Katashkina, J. I. et al. Use of the λ Red-recombineering method for genetic engineering of Pantoea ananatis. BMC Mol. Biol. 10, 34 (2009)). For transformation optimizations, we constructed pRSF-pLtetO-gfp which constitutively expresses GFP due to the absence of the tetR repressor in both E. coli and V. natriegens. We engineered pRST shuttle plasmid by fusing pCTX-Km replicon with the pir-dependent conditional replicon, R6k. To construct the conjugative suicide mariner transposon, we replaced the Tn5 transposase and Tn5 mosaic ends in pBAM1 with the mariner C9 transposase and the mariner mosaic ends from pTnFGL3 (Cameron, D. E., Urbach, J. M. & Mekalanos, J. J. A defined transposon mutant library and its use in identifying motility genes in Vibrio cholerae. Proc. Natl. Acad. Sci. U.S.A. 105, 8736-8741 (2008), Martínez-García, E., Calles, B., Arévalo-Rodríguez, M. & de Lorenzo, V. pBAM1: an all-synthetic genetic tool for analysis and construction of complex bacterial phenotypes. BMC Microbiol. 11, 38 (2011)). Our payload, the transposon DNA, consisted solely of the minimal kanamycin resistance gene required for transconjugant selection. We next performed site-directed mutagenesis on both transposon mosaic ends to introduce an MmeI cut-site, producing the plasmid pMarC9 which is also based on the pir-dependent conditional replicon, R6k. We also constructed a transposon plasmid capable of integrating a constitutively expressing GFP cassette in the genome by inserting pLtetO-GFP with either kanamycin or spectinomycin in the transposon DNA. All plasmids carrying the R6k origin was found to replicate only in either BW29427 or EC100D pir⁺/pir-116 E. coli cells. Induction systems were cloned onto the pRSF backbone. For CRISPR/Cas9 experiments, a single RSF1010 plasmid carried both Streptococcus pyogenes Cas9 and the guide RNA. dCas9 was cloned under the control of E. coli arabinose induction genes and the guide RNA under control of the constitutive J23100 promoter.

### DNA Yield

pRSF-pLtetO-gfp was transformed via electroporation into E. coli MG1655 and V. natriegens. E. coli plates were incubated at 37° C. and V. natriegens were incubated at room temperature for an equivalent time to yield approximately similar colony sizes. Three colonies from each plate was picked and grown for 5 hours at 37° C. in 3 mL of selective liquid culture (LB for E. coli and LB3 for V. natriegens) at 225 rpm. Plasmid DNA was extracted from 3 mL of culture (Qiagen Plasmid Miniprep Kit).

### CTX Vibriophage Infection

V. cholerae 0395 carrying the replicative form of CTX, CTX-Km (kanamycin resistant), was cultured overnight in LB without selection in a rotator drum at 150 rpm at 30° C. Virions were purified from cell-free supernatant (0.22 μm filtered) of overnight cultures. Replicative forms were extracted from the cells by standard miniprep (Qiagen). To test infectivity of the virions, naive V. cholerae 0395 and V. natriegens were subcultured 1:1000 in LB and LB3 respectively and mixed gently with ˜10⁶ virions. After static incubation for 30 minutes at 30° C., the mixture was plated on selective media and incubated overnight for colony formation. Replicative forms were electroporated into host strains using described protocols.

### Targeted Gene Perturbation by Cas9

All Cas9 experiments were performed using a single pRSF plasmid carrying Cas9 gene under the control of arabinose promoter, with or without GFP-targeting guide RNA. All plasmids carry carbenicillin selective marker. Wild-type V. natriegens or strain carrying genomically integrated GFP construct were grown at 37° C. overnight (LB3 or LB3+100 μg/ml kanamycin, respectively) and transformed with 50 ng of plasmid DNA using the optimized transformation protocol described above. Following 1-hour recovery in LB3 at 37° C., cells were plated on LB3+100 μg/mL carbenicillin plates and incubated overnight at 37° C. No arabinose induction was used for Cas9 experiments, as we observed low level of baseline expression using arabinose promoter.

Repression of Chromosomally-Encoded GFP with dCas9

A cassette carrying constitutive GFP expression was integration into V. natriegens by the transposon system described above. We transformed this engineered V. natriegens strain with a CRISPRi plasmid carrying dCas9 under arabinose promoter and gRNA targeting GFP. To test the repression of the chromosomally-encoded GFP with CRISPRi, we subcultured an overnight cultures 1:1000 in fresh media supplemented with or without 1 mM arabinose. We kinetically measured OD₆₀₀ and fluorescence of each culture over 12 hours in a microplate with orbital shaking at 37° C. (BioTek H1 or H4). Under these conditions, all cultures grew equivalently by OD₆₀₀.

Pooled CRISPRi Screen—Five-Member gRNA Library

dCas9 (pdCas9-bacteria was a gift from Stanley Qi; Addgene plasmid #44249) was placed under the control of tetracycline promoter, and guide RNA under constitutive J23100 promoter. Similar change in gRNA abundance was observed with or without addition of aTc, suggesting basal expression of dCas9. Five pRST plasmids (spectinomycin selective marker) each carrying a gRNA were used for targeted inhibition of the following genes: V. natriegens targeting genes lptF_{Vn} and flgC_{Vn}; targets (controls) that do not exist in the host: E. coli gene lptF_{Ec} and two for GFP. All guides were designed to target the non-template strand. An equal mix of all five plasmids, each 20 ng, was co-transformed into a dCas9 expressing V. natriegens strain. The transformation was recovered in 1 mL SOC3 media for 45 minutes at 37° C. at 225 rpm and plated on 245 mm×245 mm plates (Corning) with appropriate antibiotics. After 13 hours at 37° C., colonies were scraped in LB3. Growth competition was performed by subculturing this library 1:1000 in LB3 at 37° C. for 3 hours in baffled 250 mL flasks (Corning). At each time point, gRNA plasmid was extracted from 3 mL of culture (Qiagen Plasmid Miniprep Kit). Barcoded Illumina sequencing libraries were prepared by cycle-limited PCR with real-time PCR and sequenced with MiSeq v3 150. Resulting sequences were trimmed for the promoter and gRNA scaffold and the count of each guide sequence was first normalized by the number of sequences per time point, then expressed as a fraction of the sequence before growth competition¹⁶.

Construction, Testing, and Analysis of Genome-Wide gRNA Library

A custom python script was used to select gRNA sequences targeting the non-template strand of each RAST predicted protein-coding gene. Starting at the 5′ end of the gene, 20 bp sequences with a terminal Cas9 NGG motif on the reverse complement strand were selected. Up to 3 targets were selected for each RAST predicted gene features; each guide sequence was prefixed with a promoter and suffixed with part of the gRNA scaffold. This sequence was synthesized by the OLS process (Agilent Technologies) as an oligo library. The OLS pool was amplified by cycle-limited real-time PCR, and assembled into the pRST backbone (NEBuilder HiFi) at 5-fold molar excess with 18 bp overlap arms. 6 μL of the assembled product was mixed with 300 μL TransforMax EC100D pir+E. coli (Epicentre) and 51 μL aliquots of this mix was electroporated in 0.1 mm cuvettes with a Bio-Rad Gene Pulser electroporator at 1.8 kV, 200 Ω, 25 μF. These E. coli transformants were recovered in 6×1 mL SOC media for 60 minutes at 37° C. at 225 rpm, and plated on 245 mm×245 mm spectinomycin selective plates (Corning). After 13 hours at 37° C., ˜1.4×10⁶ colonies were scraped and plasmid DNA extracted (Qiagen HiSpeed Plasmid Maxi).

Transformation of the gRNA library into V. natriegens strains with or without dCas9 was performed as described above. Briefly, ˜600 ng of the plasmid library was mixed with 300 μL of electrocompetent cells and 53.5 μL of this mix was electroporated in 0.1 mm cuvettes with a Bio-Rad Gene Pulser electroporator at 0.4 kV, 1kΩ, 25 μF. Each transformation was recovered in 1 mL SOC3 media for 45 minutes at 37° C. at 225 rpm and plated on 245 mm×245 mm plates (Corning) with appropriate antibiotics. After 13 hours at 37° C., colonies were scraped in LB3 and stored at −80° C. as library master stocks. Growth competition of both strain libraries (guides with or without dCas9) were performed as biological duplicates, starting with dilution of the master stocks 1:1000 in LB3 with 8 hours of growth at 37° C. in baffled 250 mL flasks (Corning). The initial culture was serially diluted 3 times. At each passage, plasmid was extracted from 3 mL of culture (Qiagen Plasmid Miniprep Kit) and the culture is then diluted 1:1000 in fresh LB3 and grown at 37° C. for 4 hours. Barcoded Illumina sequencing libraries were prepared by cycle-limited real-time PCR and sequenced with MiSeq v3 150 and NextSeq v2 High Output 500/550. Resulting sequences were trimmed for the promoter and 5′-end of the gRNA scaffold. Sequencing was used to verify high coverage of our gRNA library, with representation of 99.9% (13,567 of 13,587) of all guides found in transformants. The count of each guide sequence was normalized by the number of sequences (read per million, RPM) (Martin, M. Cutadapt removes adapter sequences from high-throughput sequencing reads. EMBnet. journal 17, 10-12 (2011)). The RPM for each gene was calculated as the median of all gRNA RPMs targeting that gene and fold change for each gene was calculated as the ratio of RPM relative to the initial RPM prior to growth competition. Replicates were averaged and fold changes were normalized by setting the median for each sample to one. This final value is the relative fitness score. Note, genes above RF=2 are not displayed (6 and 18 genes for passage one and three, respectively). Significance for an RF score was determined based on the probability density function of the control experiment (e.g. guides with no dCas9). Essential genes from E. coli and V. cholerae were mapped to V. natriegens via bactNOG or COG using eggnog-mapper based on eggNOG 4.5 orthology data (Huerta-Cepas, J. et al. Fast genome-wide functional annotation through orthology assignment by eggNOG-mapper. Mol. Biol. Evol. (2017). doi:10.1093/molbev/msx148, Huerta-Cepas, J. et al. eggNOG 4.5: a hierarchical orthology framework with improved functional annotations for eukaryotic, prokaryotic and viral sequences. Nucleic Acids Res. 44, D286-93 (2016), Yamazaki, Y., Niki, H. & Kato, J.-I. Profiling of Escherichia coli Chromosome database. Methods Mol. Biol. 416, 385-389 (2008), Chao, M. C. et al. High-resolution definition of the Vibrio cholerae essential gene set with hidden Markov model-based analyses of transposon-insertion sequencing data. Nucleic Acids Res. 41, 9033-9048 (2013)). GO enrichment was computed as described above.

A non-limiting list of target genes for suppression by the methods disclosed herein is shown in Table 3.

A non-limiting list of guide RNA sequences with complementarity to target genes for suppression by the methods disclosed herein is shown in Table 4.

The contents of all references, patents and published patent applications cited throughout this application are hereby incorporated by reference in their entirety for all purposes.

TABLE 1
Major features of V. natriegens genome.

| Major features of V. natriegens genome | chr1 | chr2 |
|---|---|---|
| Size (bp) | 3,248,023 | 1,927,130 |
| G + C percentage | 45.30% | 44.70% |
| Total number of ORFs | 2884 | 1694 |
| Average ORF size (bp) | 960 | 968 |
| Number of rRNA operons (16S-23S-5S) | 10 | 1 |
| Number of tRNA | 116 | 13 |
| Genes with annotated function* | 1607 (55.7%) | 743 (43.8%) |
| Genes with unknown function** | 1277 (44.3%) | 951 (56.2%) |

*Genes annotated with a RAST category
**Genes with no RAST category annotation

TABLE 2
V. natriegens codon usage.

| Codon | AA | Fraction | Frequency | Number |
|---|---|---|---|---|
| GCA | A | 0.297 | 25.499 | 37529 |
| GCC | A | 0.16 | 13.753 | 20241 |
| GCG | A | 0.279 | 23.949 | 35248 |
| GCU | A | 0.264 | 22.657 | 33346 |
| UGC | C | 0.336 | 3.508 | 5163 |
| UGU | C | 0.664 | 6.917 | 10181 |
| GAC | D | 0.395 | 21.56 | 31732 |
| GAU | D | 0.605 | 33.05 | 48643 |
| GAA | E | 0.651 | 42.243 | 62172 |
| GAG | E | 0.349 | 22.632 | 33310 |
| UUC | F | 0.408 | 16.819 | 24754 |
| UUU | F | 0.592 | 24.364 | 35858 |
| GGA | G | 0.122 | 8.499 | 12508 |
| GGC | G | 0.324 | 22.604 | 33268 |
| GGG | G | 0.098 | 6.814 | 10029 |
| GGU | G | 0.457 | 31.901 | 46952 |
| CAC | H | 0.498 | 11.015 | 16212 |
| CAU | H | 0.502 | 11.121 | 16367 |
| AUA | I | 0.102 | 6.423 | 9454 |
| AUC | I | 0.415 | 25.998 | 38263 |
| AUU | I | 0.483 | 30.288 | 44578 |
| AAA | K | 0.685 | 35.536 | 52302 |
| AAG | K | 0.315 | 16.332 | 24037 |
| CUA | L | 0.13 | 13.323 | 19608 |
| CUC | L | 0.089 | 9.132 | 13440 |
| CUG | L | 0.223 | 22.871 | 33661 |
| CUU | L | 0.181 | 18.499 | 27227 |
| UUA | L | 0.195 | 19.94 | 29348 |
| UUG | L | 0.181 | 18.571 | 27333 |
| AUG | M | 1 | 26.992 | 39727 |
| AAC | N | 0.561 | 23.211 | 34161 |
| AAU | N | 0.439 | 18.154 | 26719 |
| CCA | P | 0.394 | 15.615 | 22983 |
| CCC | P | 0.072 | 2.874 | 4230 |
| CCG | P | 0.222 | 8.823 | 12985 |
| CCU | P | 0.311 | 12.355 | 18184 |
| CAA | Q | 0.59 | 25.924 | 38155 |
| CAG | Q | 0.41 | 18.009 | 26506 |
| AGA | R | 0.098 | 4.332 | 6376 |
| AGG | R | 0.03 | 1.313 | 1933 |
| CGA | R | 0.146 | 6.458 | 9505 |
| CGC | R | 0.266 | 11.729 | 17262 |
| CGG | R | 0.033 | 1.467 | 2159 |
| CGU | R | 0.427 | 18.836 | 27723 |
| AGC | S | 0.192 | 12.683 | 18666 |
| AGU | S | 0.181 | 11.938 | 17570 |
| UCA | S | 0.191 | 12.587 | 18526 |
| UCC | S | 0.078 | 5.125 | 7543 |
| UCG | S | 0.123 | 8.12 | 11951 |
| UCU | S | 0.235 | 15.539 | 22870 |
| ACA | T | 0.226 | 12.092 | 17797 |
| ACC | T | 0.277 | 14.826 | 21820 |
| ACG | T | 0.233 | 12.489 | 18381 |
| ACU | T | 0.265 | 14.195 | 20892 |
| GUA | V | 0.22 | 15.973 | 23509 |
| GUC | V | 0.187 | 13.608 | 20028 |
| GUG | V | 0.256 | 18.586 | 27354 |
| GUU | V | 0.336 | 24.427 | 35952 |
| UGG | W | 1 | 12.647 | 18614 |
| UAC | Y | 0.561 | 16.918 | 24900 |
| UAU | Y | 0.439 | 13.223 | 19461 |
| UAA | * | 0.65 | 2.023 | 2977 |
| UAG | * | 0.196 | 0.608 | 895 |
| UGA | * | 0.154 | 0.48 | 706 |

TABLE 3
A List of Target Genes for Suppression in V. natriegens

| Chromosome | Gene Name | Gene Annotation |
|---|---|---|
| chr1 | FIG\|691.12.PEG.2665 | ATP-dependent DNA helicase RecQ |
| chr1 | FIG\|691.12.PEG.2007 | N-acyl-L-amino acid amidohydrolase (EC 3.5.1.14) |
| chr2 | FIG\|691.12.PEG.3263 | hypothetical protein sometimes fused to ribosomal |
| | | protein S6 glutaminyl transferase |
| chr1 | FIG\|691.12.PEG.1112 | ABC transporter2C periplasmic spermidine putrescine- |
| | | binding protein PotD (TC 3.A.1.11.1) |
| chr2 | FIG\|691.12.PEG.3066 | putative protease |
| chr1 | FIG\|691.12.PEG.31 | Na+/H+ antiporter NhaP |
| chr2 | FIG\|691.12.PEG.4453 | Methyl-accepting chemotaxis protein |
| chr1 | FIG\|691.12.PEG.1180 | Transporter2C putative |
| chr1 | FIG\|691.12.PEG.854 | Biotin synthesis protein BioC |
| chr2 | FIG\|691.12.PEG.3301 | Alkaline serine protease |
| chr1 | FIG\|691.12.PEG.1054 | Glutamate Aspartate transport system permease |
| | | protein GltJ (TC 3.A.1.3.4) |
| chr1 | FIG\|691.12.PEG.2269 | Thiamin ABC transporter2C transmembrane |
| | | component |
| chr1 | FIG\|691.12.PEG.1685 | Putrescine utilization regulator |
| chr2 | FIG\|691.12.PEG.4004 | FIG01199656: hypothetical protein |
| chr1 | FIG\|691.12.PEG.2448 | Transcription elongation factor GreB |
| chr2 | FIG\|691.12.PEG.3674 | Electron transfer flavoprotein-ubiquinone |
| | | oxidoreductase (EC 1.5.5.1) |
| chr1 | FIG\|691.12.PEG.1004 | Transcriptional regulatory protein CitB2C DpiA |
| chr1 | FIG\|691.12.PEG.889 | Alcohol dehydrogenase (EC 1.1.1.1)% 3B Acetaldehyde |
| | | dehydrogenase (EC 1.2.1.10) |
| chr2 | FIG\|691.12.PEG.3789 | Malate: quinone oxidoreductase (EC 1.1.5.4) |
| chr1 | FIG\|691.12.PEG.2662 | L-lysine permease |
| chr2 | FIG\|691.12.PEG.3902 | 3-oxoacyl-[acyl-carrier protein] reductase (EC |
| | | 1.1.1.100) |
| chr1 | FIG\|691.12.PEG.1368 | hypothetical protein |
| chr1 | FIG\|691.12.PEG.1275 | hypothetical protein |
| chr2 | FIG\|691.12.PEG.3223 | D-glycerate transporter (predicted) |
| chr1 | FIG\|691.12.PEG.1349 | hypothetical protein |
| chr1 | FIG\|691.12.PEG.557 | FIG01200921: hypothetical protein |
| chr2 | FIG\|691.12.PEG.3740 | Nitrogenase FeMo-cofactor scaffold and assembly |
| | | protein NifN |
| chr1 | FIG\|691.12.PEG.1568 | FIG01200413: hypothetical protein |
| chr1 | FIG\|691.12.PEG.249 | (GlcNAc)2 ABC transporter2C ATP-binding component 2 |
| chr1 | FIG\|691.12.PEG.2849 | Acetylornithine deacetylase (EC 3.5.1.16) |
| chr2 | FIG\|691.12.PEG.3899 | two component transcriptional regulator2C LuxR family |
| chr1 | FIG\|691.12.PEG.1439 | AttF component of AttEFGH ABC transport system/ |
| | | AttG component of AttEFGH ABC transport system |
| chr2 | FIG\|691.12.PEG.3945 | hypothetical protein |
| chr1 | FIG\|691.12.PEG.2509 | ABC transporter ATP-binding protein |
| chr1 | FIG\|691.12.PEG.2547 | Dipeptide transport system permease protein DppC |
| | | (TC 3.A.1.5.2) |
| chr1 | FIG\|691.12.PEG.2413 | FIG00920623: hypothetical protein |
| chr2 | FIG\|691.12.PEG.4229 | Glucose-6-phosphate 1-dehydrogenase (EC 1.1.1.49) |
| chr2 | FIG\|691.12.PEG.4481 | Glutathione-regulated potassium-efflux system |
| | | ancillary protein KefG |
| chr1 | FIG\|691.12.PEG.890 | 5-keto-2-deoxygluconokinase (EC 2.7.1.92)/ |
| | | uncharacterized domain |
| chr1 | FIG\|691.12.PEG.2211 | Acyl-phosphate: glycerol-3-phosphate O- |
| | | acyltransferase PlsY |
| chr2 | FIG\|691.12.PEG.3468 | metal-dependent phosphohydrolase |
| chr1 | FIG\|691.12.PEG.1826 | Transcriptional activator RfaH |
| chr1 | FIG\|691.12.PEG.1735 | Menaquinone-specific isochorismate synthase (EC |
| | | 5.4.4.2) |
| chr1 | FIG\|691.12.PEG.1140 | FIG074102: hypothetical protein |
| chr1 | FIG\|691.12.PEG.2208 | Undecaprenyl-diphosphatase (EC 3.6.1.27) |
| chr2 | FIG\|691.12.PEG.3898 | Sensor histidine kinase |
| chr1 | FIG\|691.12.PEG.2404 | UDP-galactopyranose mutase (EC 5.4.99.9) |
| chr1 | FIG\|691.12.PEG.1089 | hypothetical protein |
| chr2 | FIG\|691.12.PEG.4464 | Predicted membrane fusion protein (MFP) component |
| | | of efflux pump2C membrane anchor protein YbhG |
| chr2 | FIG\|691.12.PEG.3202 | 2-deoxy-D-gluconate 3-dehydrogenase (EC 1.1.1.125) |
| chr1 | FIG\|691.12.PEG.1468 | NADH oxidoreductase hcr (EC 1.—.—.—) |
| chr1 | FIG\|691.12.PEG.1631 | hypothetical protein |
| chr1 | FIG\|691.12.PEG.255 | Ribosomal RNA small subunit methyltransferase C (EC |
| | | 2.1.1.52) |
| chr2 | FIG\|691.12.PEG.4358 | Large repetitive protein |
| chr2 | FIG\|691.12.PEG.4130 | Intracellular serine protease |
| chr1 | FIG\|691.12.PEG.2447 | hypothetical protein |
| chr1 | FIG\|691.12.PEG.2244 | 3-isopropylmalate dehydrogenase (EC 1.1.1.85) |
| chr1 | FIG\|691.12.PEG.1063 | Ribosomal RNA small subunit methyltransferase F (EC |
| | | 2.1.1.—) |
| chr1 | FIG\|691.12.PEG.1570 | Methylamine utilization protein mauG |
| chr1 | FIG\|691.12.PEG.1453 | Arginine/ornithine antiporter ArcD |
| chr1 | FIG\|691.12.PEG.2710 | Cell division protein FtsX |
| chr2 | FIG\|691.12.PEG.3639 | Malonate transporter2C MadL subunit |
| chr1 | FIG\|691.12.PEG.2442 | tRNA (guanosine(18)-2′-O)-methyltransferase (EC |
| | | 2.1.1.34) |
| chr1 | FIG\|691.12.PEG.1996 | Transcriptional regulator2C LysR family |
| chr1 | FIG\|691.12.PEG.1397 | Transcriptional regulator2C LysR family |
| chr1 | FIG\|691.12.PEG.2091 | hypothetical protein |
| chr2 | FIG\|691.12.PEG.3698 | Fusaric acid resistance domain protein |
| chr2 | FIG\|691.12.PEG.2985 | Membrane fusion component of tripartite multidrug |
| | | resistance system |
| chr1 | FIG\|691.12.PEG.186 | Aspartokinase (EC 2.7.2.4) |
| chr2 | FIG\|691.12.PEG.3330 | Heat shock protein 60 family co-chaperone GroES |
| chr1 | FIG\|691.12.PEG.692 | Tetrathionate reductase two-component response |
| | | regulator |
| chr2 | FIG\|691.12.PEG.3536 | H(+)/Cl(−) exchange transporter ClcA |
| chr1 | FIG\|691.12.PEG.115 | no significant homology |
| chr2 | FIG\|691.12.PEG.4506 | 22C4-dienoyl-CoA reductase [NADPH] (EC 1.3.1.34) |
| chr1 | FIG\|691.12.PEG.2798 | DNA mismatch repair protein MutL |
| chr1 | FIG\|691.12.PEG.343 | hypothetical protein |
| chr2 | FIG\|691.12.PEG.3722 | Predicted redox protein |
| chr2 | FIG\|691.12.PEG.4260 | Putative oxidoreductase subunit |
| chr2 | FIG\|691.12.PEG.3207 | Utilization protein for unknown catechol-siderophore X |
| chr1 | FIG\|691.12.PEG.2356 | UDP-glucose 4-epimerase (EC 5.1.3.2) |
| chr1 | FIG\|691.12.PEG.2148 | hypothetical protein |
| chr1 | FIG\|691.12.PEG.820 | hypothetical protein |
| chr1 | FIG\|691.12.PEG.754 | Outer membrane protein RomA |
| chr2 | FIG\|691.12.PEG.3138 | Membrane bound c-di-GMP receptor LapD |
| chr1 | FIG\|691.12.PEG.2368 | Capsular polysaccharide export system inner |
| | | membrane protein KpsE |
| chr2 | FIG\|691.12.PEG.2959 | FIG01200525: hypothetical protein |
| chr2 | FIG\|691.12.PEG.3493 | Transcriptional regulator2C AsnC family |
| chr2 | FIG\|691.12.PEG.3489 | Type I secretion system2C membrane fusion protein |
| | | LapC |
| chr1 | FIG\|691.12.PEG.1149 | FIG00920463: hypothetical protein |
| chr1 | FIG\|691.12.PEG.383 | hypothetical protein |
| chr2 | FIG\|691.12.PEG.4034 | SgrR2C sugar-phosphate stress2C transcriptional |
| | | activator of SgrS small RNA |
| chr2 | FIG\|691.12.PEG.4058 | N-acetylglucosamine regulated methyl-accepting |
| | | chemotaxis protein |
| chr1 | FIG\|691.12.PEG.2642 | FIG01199611: hypothetical protein |
| chr1 | FIG\|691.12.PEG.2345 | Nucleoside-diphosphate sugar epimerase/dehydratase |
| chr2 | FIG\|691.12.PEG.3572 | Na+/H+ antiporter NhaC |
| chr2 | FIG\|691.12.PEG.3972 | Hydroxymethylglutaryl-CoA reductase (EC 1.1.1.34) |
| chr2 | FIG\|691.12.PEG.4450 | conserved hypothetical membrane protein |
| chr1 | FIG\|691.12.PEG.2690 | FIG00920769: hypothetical protein |
| chr1 | FIG\|691.12.PEG.2784 | Cell wall endopeptidase2C family M23/M37 |
| chr1 | FIG\|691.12.PEG.990 | L-proline glycine betaine binding ABC transporter |
| | | protein ProX (TC 3.A.1.12.1) |
| chr1 | FIG\|691.12.PEG.1234 | hypothetical protein |
| chr2 | FIG\|691.12.PEG.3310 | 2-aminoethylphosphonate uptake and metabolism |
| | | regulator |
| chr1 | FIG\|691.12.PEG.2346 | Lipopolysaccharide biosynthesis protein RffA |
| chr1 | FIG\|691.12.PEG.2528 | NAD(FAD)-utilizing dehydrogenases |
| chr1 | FIG\|691.12.PEG.765 | Iron-regulated protein A precursor |
| chr2 | FIG\|691.12.PEG.2976 | Glyoxylase family protein |
| chr2 | FIG\|691.12.PEG.3459 | 12C4-alpha-glucan branching enzyme (EC 2.4.1.18) |
| chr2 | FIG\|691.12.PEG.4282 | Pyridoxal kinase (EC 2.7.1.35) |
| chr1 | FIG\|691.12.PEG.1329 | hypothetical protein |
| chr1 | FIG\|691.12.PEG.1019 | hypothetical protein |
| chr1 | FIG\|691.12.PEG.2279 | hypothetical protein |
| chr1 | FIG\|691.12.PEG.1864 | Flagellar hook protein FlgE |
| chr1 | FIG\|691.12.PEG.908 | Putative two-component response regulator |
| chr2 | FIG\|691.12.PEG.2934 | Outer membrane lipoprotein |
| chr1 | FIG\|691.12.PEG.1161 | FIG01199598: hypothetical protein |
| chr1 | FIG\|691.12.PEG.98 | Arginine/ornithine antiporter ArcD |
| chr2 | FIG\|691.12.PEG.3952 | diguanylate cyclase/phosphodiesterase (GGDEF & EAL |
| | | domains) with PAS/PAC sensor(s) |
| chr2 | FIG\|691.12.PEG.3270 | hypothetical protein |
| chr1 | FIG\|691.12.PEG.783 | Predicted signal-transduction protein containing |
| | | cAMP-binding and CBS domains |
| chr1 | FIG\|691.12.PEG.632 | FIG00919855: hypothetical protein |
| chr1 | FIG\|691.12.PEG.2570 | Transcriptional regulator |
| chr1 | FIG\|691.12.PEG.2239 | Probable transcriptional activator for leuABCD operon |
| chr1 | FIG\|691.12.PEG.1343 | Phage terminase large subunit GpA |
| chr1 | FIG\|691.12.PEG.728 | Tryptophan synthase beta chain (EC 4.2.1.20) |
| chr2 | FIG\|691.12.PEG.3157 | Lipoprotein releasing system transmembrane protein |
| | | LolC |
| chr1 | FIG\|691.12.PEG.2233 | Glucosamine--fructose-6-phosphate aminotransferase |
| | | [isomerizing] (EC 2.6.1.16) |
| chr1 | FIG\|691.12.PEG.396 | hypothetical protein |
| chr2 | FIG\|691.12.PEG.4118 | hypothetical protein |
| chr1 | FIG\|691.12.PEG.238 | FIG01200735: hypothetical protein |
| chr1 | FIG\|691.12.PEG.956 | Uncharacterized protein conserved in bacteria |
| chr1 | FIG\|691.12.PEG.1755 | ABC-type sugar transport system2C permease |
| | | component |
| chr2 | FIG\|691.12.PEG.4531 | FIG01199668: hypothetical protein |
| chr2 | FIG\|691.12.PEG.4414 | ABC-type transport system2C involved in lipoprotein |
| | | release2C permease component |
| chr1 | FIG\|691.12.PEG.1615 | FIG01200883: hypothetical protein |
| chr2 | FIG\|691.12.PEG.4080 | Acriflavin resistance protein |
| chr2 | FIG\|691.12.PEG.3392 | Permease of the drug/metabolite transporter (DMT) |
| | | superfamily |
| chr1 | FIG\|691.12.PEG.1243 | Malate: quinone oxidoreductase (EC 1.1.5.4) |
| chr1 | FIG\|691.12.PEG.1653 | FIG002076: hypothetical protein |
| chr1 | FIG\|691.12.PEG.346 | Tellurite resistance protein |
| chr1 | FIG\|691.12.PEG.2146 | Glutamate synthase [NADPH] large chain (EC 1.4.1.13) |
| chr1 | FIG\|691.12.PEG.944 | Putative symporter in putrescine utilization cluster |
| chr2 | FIG\|691.12.PEG.3894 | FIG01204717: hypothetical protein |

TABLE 4
A list of guide RNA complementary sequences used for target gene
suppression in V. natriegens

| | | gene | | |
|---|---|---|---|---|
| | | start | position in | |
| number | gene | (bp) | gene (%) | guideRNA sequence |
| 1 | FIG\|691.12.PEG.31 | 7 | 0.00546 | ACCTGCGGTAATGGCGATGG |
| 2 | FIG\|691.12.PEG.31 | 10 | 0.00781 | TGAACCTGCGGTAATGGCGA |
| 3 | FIG\|691.12.PEG.31 | 16 | 0.01249 | TACCATTGAACCTGCGGTAA |
| 4 | FIG\|691.12.PEG.115 | 5 | 0.00486 | GATTCGCTAATATCGCATAG |
| 5 | FIG\|691.12.PEG.115 | 27 | 0.02624 | AAATAAATACGGTTGTGGCC |
| 6 | FIG\|691.12.PEG.115 | 32 | 0.0311 | TATCAAAATAAATACGGTTG |
| 7 | FIG\|691.12.PEG.186 | 9 | 0.00758 | CCAAACTTTTGCACGATAAG |
| 8 | FIG\|691.12.PEG.186 | 10 | 0.00842 | GCCAAACTTTTGCACGATAA |
| 9 | FIG\|691.12.PEG.186 | 11 | 0.00926 | CGCCAAACTTTTGCACGATA |
| 10 | FIG\|691.12.PEG.249 | 56 | 0.05622 | TGATAGAATTGCTATTTAGC |
| 11 | FIG\|691.12.PEG.249 | 57 | 0.05723 | TTGATAGAATTGCTATTTAG |
| 12 | FIG\|691.12.PEG.249 | 85 | 0.08534 | GTCGTTGATCGCGCGCATCT |
| 13 | FIG\|691.12.PEG.255 | 18 | 0.01754 | TGGCGTTGCGCTATTTGACT |
| 14 | FIG\|691.12.PEG.255 | 38 | 0.03704 | TTCCATTGAAGTATTCCAGC |
| 15 | FIG\|691.12.PEG.255 | 76 | 0.07407 | GAATAGGTCTTCAACTTCAC |
| 16 | FIG\|691.12.PEG.343 | 98 | 0.05584 | CAATATCGACCGTCGGTTGT |
| 17 | FIG\|691.12.PEG.343 | 99 | 0.05641 | ACAATATCGACCGTCGGTTG |
| 18 | FIG\|691.12.PEG.343 | 105 | 0.05983 | TGACCGACAATATCGACCGT |
| 19 | FIG\|691.12.PEG.383 | 17 | 0.14912 | GTTTTATTGAGCGTTCTGCT |
| 20 | FIG\|691.12.PEG.383 | 58 | 0.50877 | TGTGCGCTCTCTTGCTATAT |
| 21 | FIG\|691.12.PEG.383 | 59 | 0.51754 | TTGTGCGCTCTCTTGCTATA |
| 22 | FIG\|691.12.PEG.557 | 29 | 0.03528 | CTGTGATGGATAGAGTGGCG |
| 23 | FIG\|691.12.PEG.557 | 34 | 0.04136 | GCAACCTGTGATGGATAGAG |
| 24 | FIG\|691.12.PEG.557 | 43 | 0.05231 | GCGTTCAAAGCAACCTGTGA |
| 25 | FIG\|691.12.PEG.692 | 24 | 0.03941 | TCATCGTCAACGACATAAAC |
| 26 | FIG\|691.12.PEG.692 | 106 | 0.17406 | AAAGGCTTGCCCATCAGCAA |
| 27 | FIG\|691.12.PEG.692 | 124 | 0.20361 | AATGTCTACCGCATCGAGAA |
| 28 | FIG\|691.12.PEG.754 | 130 | 0.12634 | GAGAAGCGGTGTTTTTGGTA |
| 29 | FIG\|691.12.PEG.754 | 135 | 0.1312 | TAATTGAGAAGCGGTGTTTT |
| 30 | FIG\|691.12.PEG.754 | 144 | 0.13994 | TTGATTTCATAATTGAGAAG |
| 31 | FIG\|691.12.PEG.820 | 31 | 0.14352 | TCCACTGCTGAGCTGCTGAC |
| 32 | FIG\|691.12.PEG.820 | 70 | 0.32407 | AATAGCCATGTCGATTGATT |
| 33 | FIG\|691.12.PEG.820 | 139 | 0.64352 | CTGCCCTAACACAGCAAACG |
| 34 | FIG\|691.12.PEG.854 | 60 | 0.0995 | CGTTCAGTTCTTGTGGAATG |
| 35 | FIG\|691.12.PEG.854 | 67 | 0.11111 | TACAGCTCGTTCAGTTCTTG |
| 36 | FIG\|691.12.PEG.854 | 201 | 0.33333 | CGGCATATTGCTACTGAGTC |
| 37 | FIG\|691.12.PEG.889 | 3 | 0.00111 | AGTTCTTTAATATTAGTGAC |
| 38 | FIG\|691.12.PEG.889 | 31 | 0.01149 | TTTCTTAACGCGAGTGACGA |
| 39 | FIG\|691.12.PEG.889 | 32 | 0.01187 | CTTTCTTAACGCGAGTGACG |
| 40 | FIG\|691.12.PEG.890 | 23 | 0.01205 | CGATTCGTCCCATACAAATC |
| 41 | FIG\|691.12.PEG.890 | 53 | 0.02778 | AGCCGACTTGTTGGCCGTAA |
| 42 | FIG\|691.12.PEG.890 | 62 | 0.03249 | CTAAGCGAGAGCCGACTTGT |
| 43 | FIG\|691.12.PEG.990 | 22 | 0.02183 | ACTTAGTGCCCCAATAGAAA |
| 44 | FIG\|691.12.PEG.990 | 23 | 0.02282 | TACTTAGTGCCCCAATAGAA |
| 45 | FIG\|691.12.PEG.990 | 46 | 0.04563 | ACCACTTGTCGAAAAAGCAA |
| 46 | FIG\|691.12.PEG.1004 | 56 | 0.08151 | ATTGGCTGAGGTATTTATGG |
| 47 | FIG\|691.12.PEG.1004 | 59 | 0.08588 | ACAATTGGCTGAGGTATTTA |
| 48 | FIG\|691.12.PEG.1004 | 68 | 0.09898 | CTAAGCCGGACAATTGGCTG |
| 49 | FIG\|691.12.PEG.1054 | 6 | 0.00498 | CTTAGCGATGCATCTTTAGT |
| 50 | FIG\|691.12.PEG.1054 | 37 | 0.03068 | GCTTTTATTTCCACTTGGTT |
| 51 | FIG\|691.12.PEG.1054 | 42 | 0.03483 | AATAGGCTTTTATTTCCACT |
| 52 | FIG\|691.12.PEG.1063 | 6 | 0.00418 | GCGTCTGGGATGTATACATT |
| 53 | FIG\|691.12.PEG.1063 | 20 | 0.01392 | TTTTTTCCAGAAACGCGTCT |
| 54 | FIG\|691.12.PEG.1063 | 21 | 0.01461 | ATTTTTTCCAGAAACGCGTC |
| 55 | FIG\|691.12.PEG.1089 | 31 | 0.03758 | ATCTTCACCAGCAAAAGATA |
| 56 | FIG\|691.12.PEG.1089 | 32 | 0.03879 | TATCTTCACCAGCAAAAGAT |
| 57 | FIG\|691.12.PEG.1089 | 76 | 0.09212 | ACCATTATTCTTTAACCCAG |
| 58 | FIG\|691.12.PEG.1112 | 54 | 0.05202 | TCTTGATCAGCCGCTATTGC |
| 59 | FIG\|691.12.PEG.1112 | 108 | 0.10405 | AAGTCTTCAAGAACTTCGTT |
| 60 | FIG\|691.12.PEG.1112 | 234 | 0.22543 | TTAGATACGAAATAGGTAGA |
| 61 | FIG\|691.12.PEG.1140 | 3 | 0.00377 | AAAGTAGAGCGCGATTCTAC |
| 62 | FIG\|691.12.PEG.1140 | 65 | 0.08176 | ACTGACGGTGCTGATATAAA |
| 63 | FIG\|691.12.PEG.1140 | 80 | 0.10063 | TCTTTGTCGAGATAGACTGA |
| 64 | FIG\|691.12.PEG.1149 | 33 | 0.16923 | CCTATTGAGTGCCCACAATG |
| 65 | FIG\|691.12.PEG.1149 | 70 | 0.35897 | AAACTCTTGGTCACCATTAC |
| 66 | FIG\|691.12.PEG.1149 | 83 | 0.42564 | GGCAGTCATCATAAAACTCT |
| 67 | FIG\|691.12.PEG.1180 | 6 | 0.00423 | GGGATGATGTATTTAAGATA |
| 68 | FIG\|691.12.PEG.1180 | 26 | 0.01832 | TGATTAACGGAATTATCGTT |
| 69 | FIG\|691.12.PEG.1180 | 27 | 0.01903 | ATGATTAACGGAATTATCGT |
| 70 | FIG\|691.12.PEG.1275 | 68 | 0.55285 | CATTTGTACCACTTTTCTCC |
| 71 | FIG\|691.12.PEG.1349 | 361 | 0.53245 | TATTTTATTAGCCTCTCTGT |
| 72 | FIG\|691.12.PEG.1349 | 454 | 0.66962 | ACTAGAGCTTTTACCAAGAT |
| 73 | FIG\|691.12.PEG.1349 | 479 | 0.70649 | TTAATCTATCTAGCTCTGCA |
| 74 | FIG\|691.12.PEG.1368 | 9 | 0.06977 | TAACCACCCTCAAGAATGCA |
| 75 | FIG\|691.12.PEG.1368 | 43 | 0.33333 | AAAAACAAACGCATCCGTAT |
| 76 | FIG\|691.12.PEG.1368 | 67 | 0.51938 | CAATACCGCTGCAGGTTTAA |
| 77 | FIG\|691.12.PEG.1397 | 34 | 0.03765 | AAAAGACCCTGTTTGTGCGG |
| 78 | FIG\|691.12.PEG.1397 | 37 | 0.04097 | TGTAAAAGACCCTGTTTGTG |
| 79 | FIG\|691.12.PEG.1397 | 89 | 0.09856 | CGACATATTTAGAAGTGATC |
| 80 | FIG\|691.12.PEG.1439 | 9 | 0.00367 | CCGAGTAGTGCCTTAACTAC |
| 81 | FIG\|691.12.PEG.1439 | 10 | 0.00407 | ACCGAGTAGTGCCTTAACTA |
| 82 | FIG\|691.12.PEG.1439 | 38 | 0.01548 | TAATTTGCAGTGGGTAACGA |
| 83 | FIG\|691.12.PEG.1453 | 51 | 0.11724 | GTAGGTGCCGAAATTAGCAT |
| 84 | FIG\|691.12.PEG.1453 | 69 | 0.15862 | TCCTTTTGTTGTGCGAACGT |
| 85 | FIG\|691.12.PEG.1453 | 113 | 0.25977 | TGACATCCTGCACAATCGCA |
| 86 | FIG\|691.12.PEG.1468 | 23 | 0.02178 | TACAACGGAGCGTGACGGGT |
| 87 | FIG\|691.12.PEG.1468 | 27 | 0.02557 | TCGATACAACGGAGCGTGAC |
| 88 | FIG\|691.12.PEG.1468 | 28 | 0.02652 | GTCGATACAACGGAGCGTGA |
| 89 | FIG\|691.12.PEG.1568 | 65 | 0.157 | TTTTGTTATGAACTTGTGAT |
| 90 | FIG\|691.12.PEG.1568 | 66 | 0.15942 | TTTTTGTTATGAACTTGTGA |
| 91 | FIG\|691.12.PEG.1568 | 192 | 0.46377 | TCATGAAAATCAGAGTTTGA |
| 92 | FIG\|691.12.PEG.1570 | 11 | 0.00945 | ATGCCAGCAAACCGAACTTG |
| 93 | FIG\|691.12.PEG.1570 | 85 | 0.07302 | ATTCACCGGGTTTGATGGGG |
| 94 | FIG\|691.12.PEG.1570 | 88 | 0.0756 | CTCATTCACCGGGTTTGATG |
| 95 | FIG\|691.12.PEG.1631 | 23 | 0.03633 | TGGTATTCGCATACGCATTA |
| 96 | FIG\|691.12.PEG.1631 | 24 | 0.03791 | CTGGTATTCGCATACGCATT |
| 97 | FIG\|691.12.PEG.1631 | 43 | 0.06793 | ATCTAGTTCCGTCAATGAAC |
| 98 | FIG\|691.12.PEG.1685 | 41 | 0.07387 | GAGATAAACCACGCATGGTT |
| 99 | FIG\|691.12.PEG.1685 | 46 | 0.08288 | TCGTTGAGATAAACCACGCA |
| 100 | FIG\|691.12.PEG.1685 | 100 | 0.18018 | CTGTTCAATTTGAGAGATCA |
| 101 | FIG\|691.12.PEG.1735 | 15 | 0.01208 | AAGTGTGGTTTCTCGCCAAG |
| 102 | FIG\|691.12.PEG.1735 | 30 | 0.02415 | CAATCAATCAATGAAAAGTG |
| 103 | FIG\|691.12.PEG.1735 | 66 | 0.05314 | CAATAAAACTTGGGAAAAAG |
| 104 | FIG\|691.12.PEG.1826 | 59 | 0.11776 | ACTCCACCCCTTGATTTTCA |
| 105 | FIG\|691.12.PEG.1826 | 90 | 0.17964 | ATTTTTTCGACTTCGACAGT |
| 106 | FIG\|691.12.PEG.1826 | 144 | 0.28743 | AACATATAAGAAGGGAACAG |
| 107 | FIG\|691.12.PEG.1996 | 20 | 0.02245 | CTCTTAATGCTCGGATGGAA |
| 108 | FIG\|691.12.PEG.1996 | 21 | 0.02357 | GCTCTTAATGCTCGGATGGA |
| 109 | FIG\|691.12.PEG.1996 | 25 | 0.02806 | AAAAGCTCTTAATGCTCGGA |
| 110 | FIG\|691.12.PEG.2007 | 9 | 0.00752 | TTTTTGAGTAGCGATAATTC |
| 111 | FIG\|691.12.PEG.2007 | 34 | 0.0284 | CTCACAACTGGCAACAAAAT |
| 112 | FIG\|691.12.PEG.2007 | 46 | 0.03843 | CTGAATAAATGGCTCACAAC |
| 113 | FIG\|691.12.PEG.2091 | 14 | 0.10145 | TCCCATCCAAATACCATTCA |
| 114 | FIG\|691.12.PEG.2091 | 71 | 0.51449 | GCGTATTTAACCCCTGACAG |
| 115 | FIG\|691.12.PEG.2148 | 4 | 0.01111 | ACATGGTGTTTGAGAGCGAT |
| 116 | FIG\|691.12.PEG.2148 | 21 | 0.05833 | CAATTTGGCTTATTACCACA |
| 117 | FIG\|691.12.PEG.2148 | 36 | 0.1 | TCTTGGGTGGATACGCAATT |
| 118 | FIG\|691.12.PEG.2208 | 16 | 0.0199 | CTGAATCAGAGCCAAAATAA |
| 119 | FIG\|691.12.PEG.2208 | 57 | 0.0709 | AAGTGTGCGGAGCTGGAAAT |
| 120 | FIG\|691.12.PEG.2208 | 64 | 0.0796 | CAGGATCAAGTGTGCGGAGC |
| 121 | FIG\|691.12.PEG.2211 | 31 | 0.05065 | AATAGAACCCAGTAAATAGG |
| 122 | FIG\|691.12.PEG.2211 | 34 | 0.05556 | GGAAATAGAACCCAGTAAAT |
| 123 | FIG\|691.12.PEG.2211 | 55 | 0.08987 | ACGACAAATCAACACCGCAC |
| 124 | FIG\|691.12.PEG.2244 | 25 | 0.02289 | AATACCGTCACCAGGTAGAA |
| 125 | FIG\|691.12.PEG.2244 | 33 | 0.03022 | TCAGGGCCAATACCGTCACC |
| 126 | FIG\|691.12.PEG.2244 | 50 | 0.04579 | GCGCTTGTGCCATCACTTCA |
| 127 | FIG\|691.12.PEG.2269 | 12 | 0.00753 | GCGACCCCTATGCCTAATTT |
| 128 | FIG\|691.12.PEG.2269 | 13 | 0.00816 | CGCGACCCCTATGCCTAATT |
| 129 | FIG\|691.12.PEG.2269 | 49 | 0.03076 | ACTCAGCGCAGAAAGAACAA |
| 130 | FIG\|691.12.PEG.2345 | 31 | 0.0157 | CACGATGCGCTTATTGGCTC |
| 131 | FIG\|691.12.PEG.2345 | 32 | 0.01621 | TCACGATGCGCTTATTGGCT |
| 132 | FIG\|691.12.PEG.2345 | 37 | 0.01874 | CACACTCACGATGCGCTTAT |
| 133 | FIG\|691.12.PEG.2356 | 4 | 0.00388 | TTGTTGGATTTGTTCGTATT |
| 134 | FIG\|691.12.PEG.2356 | 20 | 0.01938 | GTGATTCTAGTAACTCTTGT |
| 135 | FIG\|691.12.PEG.2356 | 42 | 0.0407 | CCAGTTACTAACCATGTTTT |
| 136 | FIG\|691.12.PEG.2368 | 42 | 0.02998 | TCAAGGTTATTAAATTGCTG |
| 137 | FIG\|691.12.PEG.2368 | 59 | 0.04211 | CTTGGCTATTTAGAAAGTCA |
| 138 | FIG\|691.12.PEG.2368 | 77 | 0.05496 | CAGCCTCAAATTTATCTGCT |
| 139 | FIG\|691.12.PEG.2404 | 28 | 0.02523 | GCATACGGCACCAAATAAAC |
| 140 | FIG\|691.12.PEG.2404 | 43 | 0.03874 | CGTCAATTCATTAGCGCATA |
| 141 | FIG\|691.12.PEG.2404 | 176 | 0.15856 | AAATAGCTTTGTCGTTTGTA |
| 142 | FIG\|691.12.PEG.2413 | 12 | 0.02516 | TTCCATCGGCGACGACGAGC |
| 143 | FIG\|691.12.PEG.2413 | 26 | 0.05451 | GGATAAGGATATTGTTCCAT |
| 144 | FIG\|691.12.PEG.2413 | 41 | 0.08595 | AAGCGATAACACCTAGGATA |
| 145 | FIG\|691.12.PEG.2442 | 5 | 0.00731 | GGATACGGTGGTAGCGTTCT |
| 146 | FIG\|691.12.PEG.2442 | 17 | 0.02485 | TCAGGACTTGGTGGATACGG |
| 147 | FIG\|691.12.PEG.2442 | 20 | 0.02924 | CTTTCAGGACTTGGTGGATA |
| 148 | FIG\|691.12.PEG.2447 | 25 | 0.08333 | TGAACATCCTACGGTTAATA |
| 149 | FIG\|691.12.PEG.2447 | 34 | 0.11333 | CGCAGAAAATGAACATCCTA |
| 150 | FIG\|691.12.PEG.2447 | 61 | 0.20333 | ACACCCTTTTAGTTCTTCTT |
| 151 | FIG\|691.12.PEG.2448 | 59 | 0.11706 | CAGGTCGCTTTTCGTTCCAG |
| 152 | FIG\|691.12.PEG.2448 | 78 | 0.15476 | GTGACTATCTTGGTGATCTC |
| 153 | FIG\|691.12.PEG.2448 | 88 | 0.1746 | GGCGGCCCAGGTGACTATCT |
| 154 | FIG\|691.12.PEG.2509 | 30 | 0.01821 | AAACCAAGCGGAGCTAAGTG |
| 155 | FIG\|691.12.PEG.2509 | 42 | 0.0255 | CAAAGCAACGATAAACCAAG |
| 156 | FIG\|691.12.PEG.2509 | 88 | 0.05343 | CGCGACAACGAGGATTGCAA |
| 157 | FIG\|691.12.PEG.2547 | 5 | 0.00529 | GAGCTGCCGCTGCATTTGAT |
| 158 | FIG\|691.12.PEG.2547 | 27 | 0.02857 | TTAAAGCGCTCCCATGCAGA |
| 159 | FIG\|691.12.PEG.2547 | 144 | 0.15238 | GGATCAGTTGGTGCCAGAAT |
| 160 | FIG\|691.12.PEG.2642 | 10 | 0.03788 | CTGGATGTCTGGGAATAAAA |
| 161 | FIG\|691.12.PEG.2642 | 20 | 0.07576 | CATCCCAGGACTGGATGTCT |
| 162 | FIG\|691.12.PEG.2642 | 21 | 0.07955 | TCATCCCAGGACTGGATGTC |
| 163 | FIG\|691.12.PEG.2662 | 13 | 0.0197 | GCTGGCCAGAGTCAGCAGGA |
| 164 | FIG\|691.12.PEG.2662 | 17 | 0.02576 | GAATGCTGGCCAGAGTCAGC |
| 165 | FIG\|691.12.PEG.2662 | 31 | 0.04697 | TAAAGCAATAAAGTGAATGC |
| 166 | FIG\|691.12.PEG.2665 | 4 | 0.00218 | AGGTTCGGCTAACAAGGTCG |
| 167 | FIG\|691.12.PEG.2665 | 10 | 0.00545 | GTCTGAAGGTTCGGCTAACA |
| 168 | FIG\|691.12.PEG.2665 | 19 | 0.01035 | TACAGGCGAGTCTGAAGGTT |
| 169 | FIG\|691.12.PEG.2690 | 9 | 0.01508 | TGAAAACCACAGTCAGGGCA |
| 170 | FIG\|691.12.PEG.2690 | 14 | 0.02345 | TGAACTGAAAACCACAGTCA |
| 171 | FIG\|691.12.PEG.2690 | 15 | 0.02513 | TTGAACTGAAAACCACAGTC |
| 172 | FIG\|691.12.PEG.2710 | 55 | 0.05676 | AGAGAAAAACCCATCGGTAT |
| 173 | FIG\|691.12.PEG.2710 | 61 | 0.06295 | ATGAACAGAGAAAAACCCAT |
| 174 | FIG\|691.12.PEG.2710 | 112 | 0.11558 | GCCTAAAGGCCGTTGCCACA |
| 175 | FIG\|691.12.PEG.2784 | 18 | 0.01592 | AAAATAGCCGAGCGGAGCAC |
| 176 | FIG\|691.12.PEG.2784 | 26 | 0.02299 | AAGTTAATAAAATAGCCGAG |
| 177 | FIG\|691.12.PEG.2784 | 61 | 0.05393 | GGAAGAGGTGAGAGGGAACG |
| 178 | FIG\|691.12.PEG.2798 | 18 | 0.00886 | ATCTGGTTGGCGAGCCTGGC |
| 179 | FIG\|691.12.PEG.2798 | 22 | 0.01083 | TGCTATCTGGTTGGCGAGCC |
| 180 | FIG\|691.12.PEG.2798 | 31 | 0.01526 | CTCCCCTGCTGCTATCTGGT |
| 181 | FIG\|691.12.PEG.2849 | 9 | 0.00792 | TCATAGACCTCTAGAAACGT |
| 182 | FIG\|691.12.PEG.2849 | 46 | 0.04046 | GTCGGTGGAGCTGATCGAAG |
| 183 | FIG\|691.12.PEG.2849 | 61 | 0.05365 | TTGGTCCCATTTTGGGTCGG |
| 184 | FIG\|691.12.PEG.2959 | 58 | 0.16667 | CATTTGAGTGATGTGCATTT |
| 185 | FIG\|691.12.PEG.2959 | 95 | 0.27299 | ACTCATGTAAGCTCACACCG |
| 186 | FIG\|691.12.PEG.2959 | 125 | 0.3592 | GGGGTGAACTACCACGTAAT |
| 187 | FIG\|691.12.PEG.2985 | 93 | 0.10473 | ACCTGACCATCTCGCGTCCA |
| 188 | FIG\|691.12.PEG.2985 | 94 | 0.10586 | TACCTGACCATCTCGCGTCC |
| 189 | FIG\|691.12.PEG.2985 | 121 | 0.13626 | AGGCGCAACTTTGATGACAT |
| 190 | FIG\|691.12.PEG.3066 | 24 | 0.01553 | TTATGCAGCTCCGGGTCCGG |
| 191 | FIG\|691.12.PEG.3066 | 27 | 0.01748 | TTGTTATGCAGCTCCGGGTC |
| 192 | FIG\|691.12.PEG.3066 | 32 | 0.02071 | CTATCTTGTTATGCAGCTCC |
| 193 | FIG\|691.12.PEG.3138 | 40 | 0.02151 | ACGCTGCTGTTGCTCCAGAC |
| 194 | FIG\|691.12.PEG.3138 | 79 | 0.04247 | TGCCAAACCGACAGTATTGA |
| 195 | FIG\|691.12.PEG.3138 | 108 | 0.05806 | TTATCTTTGTCTTTTAGGTA |
| 196 | FIG\|691.12.PEG.3202 | 20 | 0.02625 | CAATGGCTACTTTGCCTTCA |
| 197 | FIG\|691.12.PEG.3202 | 37 | 0.04856 | AGTGTCACAACCCGTAACAA |
| 198 | FIG\|691.12.PEG.3202 | 68 | 0.08924 | CTAAACCAAGCGCCATACCT |
| 199 | FIG\|691.12.PEG.3207 | 12 | 0.0155 | ACGACAAGTTCTTTAGGTTC |
| 200 | FIG\|691.12.PEG.3207 | 18 | 0.02326 | TGCGTAACGACAAGTTCTTT |
| 201 | FIG\|691.12.PEG.3207 | 55 | 0.07106 | AGTAAGGCGCTGCATGTTTG |
| 202 | FIG\|691.12.PEG.3223 | 5 | 0.0037 | CCCCGAGCAGGATTAATATA |
| 203 | FIG\|691.12.PEG.3223 | 17 | 0.01259 | TAAACGCGATCACCCCGAGC |
| 204 | FIG\|691.12.PEG.3223 | 49 | 0.0363 | ATGTAACTTAAATTTGGTGG |
| 205 | FIG\|691.12.PEG.3263 | 28 | 0.06527 | CTCTGGTAAGCAGATTGCTT |
| 206 | FIG\|691.12.PEG.3263 | 45 | 0.1049 | AGATGGGGAATTCCTAACTC |
| 207 | FIG\|691.12.PEG.3263 | 60 | 0.13986 | TCAATACGAGCTTCAAGATG |
| 208 | FIG\|691.12.PEG.3301 | 46 | 0.02262 | AGTCGTTAGCGCAACGGCCA |
| 209 | FIG\|691.12.PEG.3301 | 52 | 0.02557 | CAAAGAAGTCGTTAGCGCAA |
| 210 | FIG\|691.12.PEG.3301 | 90 | 0.04425 | GATTCATCAAATCCCATCGG |
| 211 | FIG\|691.12.PEG.3330 | 12 | 0.04124 | ACAAGCAGCTTGTCATTTAA |
| 212 | FIG\|691.12.PEG.3330 | 103 | 0.35395 | TGCAATGACCTTACCTCGGT |
| 213 | FIG\|691.12.PEG.3330 | 107 | 0.3677 | CAACTGCAATGACCTTACCT |
| 214 | FIG\|691.12.PEG.3468 | 14 | 0.02191 | CTAGAAATAGGGGTTCTACT |
| 215 | FIG\|691.12.PEG.3468 | 24 | 0.03756 | TGCATGAATTCTAGAAATAG |
| 216 | FIG\|691.12.PEG.3468 | 25 | 0.03912 | CTGCATGAATTCTAGAAATA |
| 217 | FIG\|691.12.PEG.3489 | 6 | 0.00567 | CAGCGCGCGAACTTTTGGTC |
| 218 | FIG\|691.12.PEG.3489 | 11 | 0.01039 | TAATCCAGCGCGCGAACTTT |
| 219 | FIG\|691.12.PEG.3489 | 56 | 0.05288 | TTAGAAAGTAAGCAAAGACA |
| 220 | FIG\|691.12.PEG.3493 | 29 | 0.0636 | GAGTGGCGTCTTTTTGAATC |
| 221 | FIG\|691.12.PEG.3493 | 46 | 0.10088 | GAGATCAGCCGTCGTCAGAG |
| 222 | FIG\|691.12.PEG.3493 | 90 | 0.19737 | ATTCGCCTTGCGCATGGAGA |
| 223 | FIG\|691.12.PEG.3536 | 4 | 0.00285 | CTTCACAATTCTCTCTCTCT |
| 224 | FIG\|691.12.PEG.3536 | 45 | 0.03205 | AACTGGTTAATCGCATCTTT |
| 225 | FIG\|691.12.PEG.3536 | 62 | 0.04416 | TCGACCCTCGAGAGATAAAC |
| 226 | FIG\|691.12.PEG.3572 | 15 | 0.01031 | GCCATGGTGAAGGGTATTTT |
| 227 | FIG\|691.12.PEG.3572 | 24 | 0.01649 | GGAAAAATTGCCATGGTGAA |
| 228 | FIG\|691.12.PEG.3572 | 25 | 0.01718 | CGGAAAAATTGCCATGGTGA |
| 229 | FIG\|691.12.PEG.3639 | 87 | 0.22481 | CCAACACCCCCAACATTTGA |
| 230 | FIG\|691.12.PEG.3639 | 215 | 0.55556 | CCGCCATGGCTACAACGATT |
| 231 | FIG\|691.12.PEG.3639 | 216 | 0.55814 | GCCGCCATGGCTACAACGAT |
| 232 | FIG\|691.12.PEG.3674 | 33 | 0.02033 | CAGGCAGCAGACAAGCCCGC |
| 233 | FIG\|691.12.PEG.3674 | 52 | 0.03204 | TGCCAACTGTTTAATGCGGC |
| 234 | FIG\|691.12.PEG.3674 | 56 | 0.0345 | CCATTGCCAACTGTTTAATG |
| 235 | FIG\|691.12.PEG.3698 | 23 | 0.01326 | CGATAAGTGTCCCCAGAATA |
| 236 | FIG\|691.12.PEG.3698 | 110 | 0.06344 | ATAGACCAACCCATAATGCC |
| 237 | FIG\|691.12.PEG.3698 | 139 | 0.08016 | ACCATGGATAACATTACCTG |
| 238 | FIG\|691.12.PEG.3722 | 16 | 0.03419 | TTGGCGTTGCCACTGAATAG |
| 239 | FIG\|691.12.PEG.3722 | 35 | 0.07479 | CGCTGAAAATCTCGCTTGCT |
| 240 | FIG\|691.12.PEG.3722 | 111 | 0.23718 | ACATGAGGGGACGGAGAAGC |
| 241 | FIG\|691.12.PEG.3740 | 9 | 0.00656 | AGTGGTGAATTTTGTTTGAT |
| 242 | FIG\|691.12.PEG.3740 | 27 | 0.01969 | TTTAACGGCTGGGTAACCAG |
| 243 | FIG\|691.12.PEG.3740 | 37 | 0.02699 | CGGGCTCGTTTTTAACGGCT |
| 244 | FIG\|691.12.PEG.3789 | 32 | 0.01998 | TTTCTCTGTTAGAACTGAGC |
| 245 | FIG\|691.12.PEG.3898 | 17 | 0.01227 | TGATGTTTATTGTAAGCCTG |
| 246 | FIG\|691.12.PEG.3898 | 57 | 0.04113 | ATGACCACCACCACAATCAA |
| 247 | FIG\|691.12.PEG.3898 | 125 | 0.09019 | TGAGTCGTGTTAATGCGACT |
| 248 | FIG\|691.12.PEG.3899 | 19 | 0.03016 | TTGCATTAACCTATGATCAT |
| 249 | FIG\|691.12.PEG.3899 | 86 | 0.13651 | TGCCATTTCCGACGCAATCG |
| 250 | FIG\|691.12.PEG.3899 | 133 | 0.21111 | AAGCAGCACGTCTGGTTTTA |
| 251 | FIG\|691.12.PEG.3902 | 50 | 0.06803 | CAAATCGTTCCACAATCGCT |
| 252 | FIG\|691.12.PEG.3902 | 100 | 0.13605 | AGTTTGAGCGTTAACATCTA |
| 253 | FIG\|691.12.PEG.3902 | 143 | 0.19456 | CGACTGGTTTAACATTTTCG |
| 254 | FIG\|691.12.PEG.3945 | 96 | 0.8 | CAAACTGGGTTGATTGAGTT |
| 255 | FIG\|691.12.PEG.3972 | 3 | 0.00238 | GTATTATCTAGGTTTAACTT |
| 256 | FIG\|691.12.PEG.3972 | 14 | 0.01108 | AGTGTTGGGCGGTATTATCT |
| 257 | FIG\|691.12.PEG.3972 | 25 | 0.01979 | AGAAAGAGCGGAGTGTTGGG |
| 258 | FIG\|691.12.PEG.4004 | 14 | 0.01587 | ACACTAATGACAAAACTACA |
| 259 | FIG\|691.12.PEG.4004 | 15 | 0.01701 | AACACTAATGACAAAACTAC |
| 260 | FIG\|691.12.PEG.4004 | 45 | 0.05102 | AACCAGTCCATAGCTTGGAC |
| 261 | FIG\|691.12.PEG.4034 | 9 | 0.0053 | TCAAACTGAACACGTAGGCG |
| 262 | FIG\|691.12.PEG.4034 | 14 | 0.00824 | GTGTTTCAAACTGAACACGT |
| 263 | FIG\|691.12.PEG.4034 | 167 | 0.09835 | GCTTTCCTCGACCCGCGGCT |
| 264 | FIG\|691.12.PEG.4058 | 32 | 0.02309 | TTAGAGCCAGCAGAAACAGT |
| 265 | FIG\|691.12.PEG.4058 | 71 | 0.05123 | GGTTGATTTCGTTGTAGTAA |
| 266 | FIG\|691.12.PEG.4058 | 92 | 0.06638 | TGATTGCTTGATGTTCAAAC |
| 267 | FIG\|691.12.PEG.4130 | 6 | 0.00335 | AGTAGCAAAGTAACTAAAAT |
| 268 | FIG\|691.12.PEG.4130 | 57 | 0.03183 | GGAGGCGCTGCTATCGTGAT |
| 269 | FIG\|691.12.PEG.4130 | 75 | 0.04188 | ACAATATATCCTCCGGAAGG |
| 270 | FIG\|691.12.PEG.4229 | 9 | 0.00599 | ATCACGATGCTGCTTTTTTC |
| 271 | FIG\|691.12.PEG.4229 | 72 | 0.0479 | GCGTATAAGTGATATAACGC |
| 272 | FIG\|691.12.PEG.4229 | 73 | 0.04857 | TGCGTATAAGTGATATAACG |
| 273 | FIG\|691.12.PEG.4260 | 118 | 0.07579 | TAATACACCTTTATTCATGT |
| 274 | FIG\|691.12.PEG.4260 | 191 | 0.12267 | CTGCTTTAATAACAACAAAT |
| 275 | FIG\|691.12.PEG.4260 | 280 | 0.17983 | CGAACCGCCTAATCCAGCAG |
| 276 | FIG\|691.12.PEG.4358 | 39 | 0.01148 | GTTCCACTCACCAATGTTCT |
| 277 | FIG\|691.12.PEG.4358 | 70 | 0.02061 | AGAAAAAGCGGTCAACGCAG |
| 278 | FIG\|691.12.PEG.4358 | 82 | 0.02415 | GGGAAAAGAGAGAGAAAAAG |
| 279 | FIG\|691.12.PEG.4450 | 7 | 0.01042 | TTCCACCCAACGAGGAAGTC |
| 280 | FIG\|691.12.PEG.4450 | 15 | 0.02232 | GCGCCATATTCCACCCAACG |
| 281 | FIG\|691.12.PEG.4450 | 49 | 0.07292 | ATTGACGCAGCCTGCAAGAA |
| 282 | FIG\|691.12.PEG.4453 | 25 | 0.01335 | GAGCAACAAAATTGAAGAGG |
| 283 | FIG\|691.12.PEG.4453 | 28 | 0.01496 | AATGAGCAACAAAATTGAAG |
| 284 | FIG\|691.12.PEG.4453 | 89 | 0.04754 | TATGTTCGCTGGTTTGAGCT |
| 285 | FIG\|691.12.PEG.4464 | 22 | 0.02321 | AGTCAGTAGAGCAATAATTA |
| 286 | FIG\|691.12.PEG.4464 | 95 | 0.10021 | AGGTTGCGGTGAAGGTTACG |
| 287 | FIG\|691.12.PEG.4464 | 103 | 0.10865 | CTCATTTGAGGTTGCGGTGA |
| 288 | FIG\|691.12.PEG.4481 | 4 | 0.00647 | GAGAATTAATACTTTTTTAT |
| 289 | FIG\|691.12.PEG.4481 | 35 | 0.05663 | CTTCTGAACGATGTTGAGAA |
| 290 | FIG\|691.12.PEG.4481 | 36 | 0.05825 | ACTTCTGAACGATGTTGAGA |
| 291 | FIG\|691.12.PEG.4506 | 21 | 0.01907 | TGGAATCGACCTACTTTGAG |
| 292 | FIG\|691.12.PEG.4506 | 41 | 0.03724 | TAACGATACGGTTAGCTAAC |
| 293 | FIG\|691.12.PEG.4506 | 53 | 0.04814 | TCATTGGCGGCATAACGATA |

### Other Embodiments

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference in their entirety for all purposes to the same extent as if each individual publication or patent application were specifically and individually indicated to be so incorporated by reference.

1. A method of altering a target nucleic acid sequence within a non-E. coli cell comprising
providing a cell with a functioning beta-like recombinase and a donor nucleic acid sequence, wherein the donor nucleic acid sequence is inserted into the target nucleic acid sequence as a result of the functioning beta-recombinase.

2. The method of claim 1 wherein the non-E. coli cell is Vibrio natriegens.

3. The method of claim 1 wherein the beta-like recombinase is identified in a horizontal gene transfer element such as a phage.

4. The method of claim 1 wherein the beta-like recombinase is identified in a horizontal gene transfer element such as an Integrative and Conjugative Element (ICE).

5. The method of claim 1 wherein the beta-like recombinase is identified in a horizontal gene transfer element such as a conjugative plasmid.

6. The method of claim 1 wherein the beta-like recombinase is identified in a horizontal gene transfer element such as a Vibrio spp. phage.

7. The method of claim 1 wherein the beta-like recombinase is identified in a horizontal gene transfer element such as a Vibrio spp. Integrative and Conjugative Element (ICE).

8. The method of claim 1 wherein the beta-like recombinase is s065.

9. The method of claim 1 wherein additional recombination assisting proteins are provided to the cell.

10. The method of claim 1 wherein additional recombination assisting proteins are provided to the cell including the exonuclease s066, a host nuclease inhibitor such as gam, and a single-strand DNA binding (SSB) protein s064 (Uniprot: A0A0X1L3H7).

11. The method of claim 1 wherein additional recombination assisting proteins are provided to the cell including s066, and gam to create a single-stranded intermediate from a double stranded nucleic acid donor.

12. The method of claim 1 wherein the donor nucleic acid sequence is introduced into the cell as a single stranded nucleic acid.

13. The method of claim 1 wherein the donor nucleic acid sequence is introduced into the cell as a double stranded nucleic acid.

14. The method of claim 1 wherein the cell has been genetically modified to include a foreign nucleic acid sequence encoding the recombinase.

15. The method of claim 1 wherein the cell has been genetically modified to include a foreign nucleic acid sequence encoding the recombinase, exonuclease, host nuclease inhibitor, and SSB.

16. The method of claim 1 wherein the cell has been genetically modified to include a foreign nucleic acid sequence encoding the s065, exonuclease, host nuclease inhibitor and SSB.

17. The method of claim 1 wherein the cell has been genetically modified to include a foreign nucleic acid sequence encoding the s065, s066, s064, and host nuclease inhibitor.

18. The method of claim 1 wherein the cell has been genetically modified to include a foreign nucleic acid sequence encoding the s065, s066, s064, and gam.

19. The method of claim 1 wherein the donor nucleic acid sequence is provided to the cell by electroporation.

20.-37. (canceled)

38. A method of altering a target nucleic acid sequence within a Vibrio natriegens cell comprising
providing the Vibrio natriegens cell with a functioning s065 recombinase and a donor nucleic acid sequence, wherein the donor nucleic acid sequence is inserted into the target nucleic acid sequence as a result of the functioning s065.

39. The method of claim 38 wherein additional recombination assisting proteins are provided to the cell.

40. The method of claim 38 wherein additional recombination assisting proteins are provided to the cell including the exonuclease s066, and a host nuclease inhibitor such as gam.

41. The method of claim 38 wherein additional recombination assisting proteins are provided to the cell including s066, and gam to create a single-stranded intermediate from a double stranded nucleic acid donor.

42. The method of claim 38 wherein the donor nucleic acid sequence is introduced into the cell as a single stranded nucleic acid.

43. The method of claim 38 wherein the donor nucleic acid sequence is introduced into the cell as a double stranded nucleic acid.

44. The method of claim 38 wherein the cell has been genetically modified to include a foreign nucleic acid sequence encoding the recombinase.

45. The method of claim 38 wherein the cell has been genetically modified to include a foreign nucleic acid sequence encoding the recombinase, exonuclease, and host nuclease inhibitor.

46. The method of claim 38 wherein the cell has been genetically modified to include a foreign nucleic acid sequence encoding the s065, exonuclease, host nuclease inhibitor, and SSB.

47. The method of claim 38 wherein the cell has been genetically modified to include a foreign nucleic acid sequence encoding the s065, s066, s064, and host nuclease inhibitor.

48. The method of claim 38 wherein the cell has been genetically modified to include a foreign nucleic acid sequence encoding the s065, s066, s064, and gam.

49. The method of claim 38 wherein the donor nucleic acid sequence is provided to the cell by electroporation.

50. A genetically modified Vibrio natriegens cell comprising a foreign nucleic acid sequence encoding a beta-like recombinase.

51. The genetically modified Vibrio natriegens cell of claim 50 wherein the beta-like recombinase is s065.

52. The genetically modified Vibrio natriegens cell of claim 50 further including a foreign donor nucleic acid sequence.

53. The genetically modified Vibrio natriegens cell of claim 50 further including a foreign donor nucleic acid sequence inserted into plasmid or genomic DNA within the Vibrio natriegens cell.

54. A method of modulating expression of a target nucleic acid sequence within a non-E. coli cell comprising
providing the cell with a guide RNA comprising a portion that is complementary to all or a portion of the target nucleic acid sequence, and
providing the cell a Cas protein,
wherein the guide RNA and the Cas protein co-localize at the target nucleic acid sequence and wherein the Cas protein modulate the expression of the target nucleic acid sequence.

55. The method of claim 54 wherein the non-E. coli cell is Vibrio natriegens.

56.-66. (canceled)

67. A method of altering a target nucleic acid sequence within a non-E. coli cell comprising
providing the cell with a guide RNA comprising a portion that is complementary to all or a portion of the target nucleic acid sequence,
providing the cell a Cas protein, and
providing the cell a donor nucleic acid sequence,
wherein the guide RNA and the Cas protein co-localize at the target nucleic acid sequence, wherein the Cas protein cleaves the target nucleic acid sequence and the donor nucleic acid sequence is inserted into the target nucleic acid sequence in a site specific manner.

68. The method of claim 67 wherein the non-E. coli cell is Vibrio natriegens.

69.-81. (canceled)

82. A nucleic acid construct encoding a guide RNA comprising a portion that is complementary to a target nucleic acid sequence in Vibrio natriegens.

83. (canceled)

84. A nucleic acid construct encoding a donor nucleic acid sequence for insertion into a target nucleic acid sequence in Vibrio natriegens.

85. A non-E. coli cell comprising
a guide RNA comprising a portion that is complementary to all or a portion of the target nucleic acid sequence, and
a Cas protein,
wherein the guide RNA and the Cas protein co-localize at the target nucleic acid sequence and modulates the expression of the target nucleic acid sequence in the cell.

86. The method of claim 85 wherein the non-E. coli cell is Vibrio natriegens.

87. A non-E. coli cell comprising
a guide RNA comprising a portion that is complementary to all or a portion of the target nucleic acid sequence,
a Cas protein, and
a donor nucleic acid sequence,
wherein the guide RNA and the Cas protein co-localize at the target nucleic acid sequence, wherein the Cas protein cleaves the target nucleic acid sequence and the donor nucleic acid sequence is inserted into the target nucleic acid sequence in a site specific manner.

88. The cell of claim 87 wherein the non-E. coli cell is Vibrio natriegens.

89. A method of improving the growth rate of a non-E. coli cell comprising
suppressing the expression of a target gene of the non-E. coli cell.

90.-92. (canceled)

93. The method of claim 89 wherein the non-E. coli cell is Vibrio natriegens.

94.-102. (canceled)

103. The method of claim 93 wherein the target gene comprises ATP-dependent DNA helicase RecQ, N-acyl-L-amino acid amidohydrolase, a hypothetical protein fused to ribosomal protein S6 glutaminyl transferase, ABC transporter2C periplasmic spermidine putrescine-binding protein PotD, a putative protease, Na+/H+ antiporter NhaP, methyl-accepting chemotaxis protein, transporter2C putative, biotin synthesis protein BioC, alkaline serine protease, glutamate aspartate transport system permease protein GltJ, thiamin ABC transporter2C transmembrane component, or putrescine utilization regulator.

104.-105. (canceled)
